(19) United States
(12) Patent Application Publication
(10) Pub. No.: US 2025/0118405 A1
(43) Pub. Date: Apr. 10, 2025

(54) PREDICTING PATIENT RESPONSE

(71) Applicant: ONCOHOST LTD., Binyamina (IL)

(72) Inventors: Coren LAHAV, Tel Aviv (IL); Itamar SELA, Ramat-Gan (IL); Yehonatan ELON, Jerusalem (IL); Michal HAREL, Hod Hasharon (IL); Eyal JACOB, Haifa (IL)

(21) Appl. No.: 18/682,220

(22) PCT Filed: Aug. 11, 2022
(86) PCT No.: PCT/IL2022/050881

§ 371 Date: Feb. 8, 2024

Related U.S. Application Data
(60) Provisional application No. 63/231,770, filed on Aug. 11, 2021.
(60) Provisional application No. 63/324,116, filed on Mar. 27, 2022.

(51) Int. Cl. G16H 20/10; G06N 20/00; G16B 20/20; G16H 50/70

(52) U.S. Cl. CPC G16H 20/10; G06N 20/00; G16B 20/20; G16H 50/70

(57) ABSTRACT

Methods of predicting response of a subject suffering from a disease to a therapy, comprising calculating a resistance score for factors expressed by the subject, classifying a factor as a resistance-associated factor based on the resistance score, wherein a number of resistance-associated factors above a predetermined threshold indicates a subject is predicted to be resistant to the therapy, are provided. Methods of predicting response based on the number of resistance-associated factors and at least one clinical parameter are also provided.

### CROSS REFERENCE TO RELATED APPLICATIONS

This application claims the benefit of priority of U.S. Provisional Patent Application No. 63/231,770, filed on Aug. 11, 2021, and U.S. Provisional Patent Application No. 63/324,116, filed on Mar. 27, 2022, the contents of which are all incorporated herein by reference in their entirety.

### FIELD OF THE INVENTION

The present invention is in the field of patient-specific diagnostics.

### BACKGROUND OF THE INVENTION

One of the major complications in oncology is resistance to therapy. Many studies have focused on the involvement of mutations and epigenetic changes in tumor cells in conferring drug resistance. However, in recent years, studies have indicated that in response to almost any type of anti-cancer therapy, the patient (i.e., the host) may generate pro-tumorigenic and pro-metastatic effects. This phenomenon, called host-response, is the physiological reaction of the patient to the cancer therapy that potentially counteracts the anti-tumor activity of the treatment.

Lung cancer displays the highest death rate among the different cancer types, with approximately 2.1 million lung cancer cases and 1.8 million deaths in 2018 worldwide. More than 85% of the lung cancer cases are classified as non-small lung cancer (NSCLC) of which the two most common histological subtypes are lung adenocarcinoma and lung squamous cell carcinoma. The treatment of NSCLC has shifted from the use of mainly chemotherapy to more personalized approaches. Currently, patient-specific genetic alterations in tumor cells determine eligibility for receiving targeted agents. In particular, the status of programmed death ligand-1 (PD-L1) expression levels in the tumor determines eligibility for receiving this immunotherapy.

Immunotherapy is a type of treatment based on immune response modulation. Currently, one of the most common immunotherapy approaches is the use of immune checkpoint inhibitors (ICI), which target regulators of the immune system in order to stimulate it. At present, there are several approved ICIs in the form of monoclonal antibodies targeting the immune checkpoint proteins CTLA4, PD-1, and PD-L1. ICI treatments are approved for treatment in multiple cancer types, including melanoma and NSCLC.

Several limitations exist for these therapeutic agents when used as monotherapy, with objective responses observed in only 20-30% of patients. In addition, immune mechanisms involved in the response to these therapeutic interventions remain poorly elucidated. Thus, advanced proteomic technologies enabling an easy and non-invasive means for the discovery of blood-based protein biomarkers promise to identify host and tumor changes associated with immunotherapy response/non-response and uncover biological mechanisms underlying host related primary resistance. A method of determining patient-specific response to therapy, and in particular immunotherapy, is greatly needed.

### SUMMARY OF THE INVENTION

The present invention provides methods of predicting response of a subject to a therapy. Methods of predicting response to a therapy of a subject suffering from a disease, comprising calculating a resistance score for proteins expressed by the subject, classifying a protein as a resistance-associated protein (RAP) based on the resistance score, wherein a number of resistance-associated proteins beyond a predetermined threshold indicates a subject is predicted to be resistant to the therapy, are provided. Methods of predicting response based on the number of resistance-associated factors and at least one clinical parameter are also provided.

According to a first aspect, there is provided a method of predicting response of a subject suffering from a disease to a therapy, the method comprising:
a. receiving protein expression levels for a plurality of factors
   i. in a population of subjects suffering from the disease and known to respond to the therapy (responders);
   ii. in a population of subjects suffering from the disease and known to not respond to the therapy (non-responders); and
   iii. in the subject;
b. calculate for factors of the plurality of factors a resistance score, where the resistance score is based on the similarity of the factor expression level in the subject to the factor expression level in the responders and the similarity of the factor expression level in the subject to the non-responders; and
c. classify a factor of the plurality of factors with a resistance score beyond a predetermined threshold as a resistance-associated factor;
wherein a subject with a number of resistance-associated factors beyond a predetermined number is predicted to be resistant to the therapy and a subject with a number of resistance-associated factors at or below a predetermined number is predicted to respond to the therapy;
thereby predicting the response of a subject to a therapy.

According to another aspect, there is provided a method of predicting response of a subject suffering from a disease to a therapy, the method comprising:
a. receiving factor expression levels for a plurality of factors
   i. in a population of subjects suffering from the disease and known to respond to the therapy (responders);
   ii. in a population of subjects suffering from the disease and known to not respond to the therapy (non-responders); and
   iii. in the subject;
b. calculate for factors of the plurality of factors a resistance score, where the resistance score is based on the similarity of the factor expression level in the subject to the factor expression level in the responders and the similarity of the factor expression level in the subject to the non-responders;
c. classify a factor of the plurality of factors with a resistance score beyond a predetermined threshold as a resistance-associated factor;
d. sum the number of resistance-associated factors present for the subject; and
e. applying a trained machine learning algorithm to the number of resistance-associated factors and at least one clinical parameter of the subject, wherein the trained machine learning algorithm outputs a final resistance score and a final resistance score beyond a predetermined threshold indicates the subject is resistant to the therapy;
thereby predicting the response of a subject to a therapy.

According to another aspect, there is provided a method of predicting response of a subject suffering from a disease to a therapy, the method comprising:
a. receiving factor expression levels for a plurality of factors
   i. in a population of subjects suffering from the disease and known to respond to the therapy (responders);
   ii. in a population of subjects suffering from the disease and known to not respond to the therapy (non-responders); and
   iii. in the subject;
b. calculate for factors of the plurality of factors a resistance score, where the resistance score is based on the similarity of the factor expression level in the subject to the factor expression level in the responders and the similarity of the factor expression level in the subject to the non-responders and wherein the calculating comprises applying a machine learning algorithm trained on a training set comprising the received factor expression levels in responders and non-responders and the sex of each of the responders and non-responders to individual received factor expression levels from the subject and the subject's sex and wherein the machine learning algorithm outputs the resistance score; and
c. sum the calculated resistance scores to produce a total resistance score; wherein a subject with a total resistance score beyond a predetermined threshold is predicted to be resistant to said therapy;
thereby predicting the response of a subject to a therapy.

According to another aspect, there is provided a method comprising:
at a training stage, training a machine learning algorithm on a training set comprising:
(i) the number of resistance-associated factors expressed in samples from subjects suffering from a disease and known to be responsive to a therapy and the number of resistance-associated factors expressed in samples from subjects suffering from the disease and known to be non-responsive to the therapy;
(ii) at least one clinical parameter of the subjects known to be responsive and the subjects known to be non-responsive; and
(iii) labels associated with the responsiveness of the subjects suffering from the disease;
to produce a trained machine learning algorithm, wherein the trained machine learning algorithm is trained to predict responsiveness of subjects suffering from the disease to the therapy.

According to another aspect, there is provided a method comprising:
at a training stage, training a machine learning algorithm on a training set comprising:
(i) factor expression levels of resistance-associated factors in samples from subjects suffering from a disease and known to be responsive to a therapy and factor expression levels of resistance-associated factors in samples from subjects suffering from the disease and known to be non-responsive to the therapy;
(ii) at least one clinical parameter of the subjects known to be responsive and the subjects known to be non-responsive; and
(iii) labels associated with the responsiveness of the subjects suffering from the disease;
to produce a trained machine learning algorithm, wherein the trained machine learning algorithm is trained to predict activity of a resistance-associated factor in a subject.

According to some embodiments, the number of resistance-associated factors and the at least one clinical parameter are labeled with the labels.

According to some embodiments, the final resistance score predetermined threshold is 0.2 and a resistance score above 0.2 indicates the subject is resistant to the therapy or the final resistance score is converted to a response score by the equation (1-final resistance score) and wherein a response score above a predetermined threshold indicates the subject is responsive to therapy, optionally wherein the response score predetermined threshold is 0.8.

According to some embodiments, the resistance-associated factors in each subject are determined by a method comprising:
a. receiving factor expression levels for a plurality of factors
   i. in a population of subjects suffering from the disease and known to respond to the therapy (responders);
   ii. in a population of subjects suffering from the disease and known to not respond to the therapy (non-responders); and
   iii. in the each subject;
b. calculate for each factor of the plurality of proteins a resistance score, where the resistance score is based on the similarity of the factor expression level in the each subject to the factor expression level in the responders and the similarity of the factor expression level in the subject to the non-responders; and
c. classify a factor of the plurality of factors with a resistance score above a predetermined threshold as a resistance-associated factor.

According to some embodiments, the method comprises before (b) selecting a subset of the plurality of factors, wherein the subset comprises factors that best differentiate between the responders and non-responders, and wherein the calculating is for each factor of the subset.

According to some embodiments, the selecting comprises applying a statistical test to the received factor expression levels, optionally wherein the statistical test is a Kolmogorov-Smirnov test.

According to some embodiments, the calculating comprises applying a machine learning algorithm trained on a training set comprising the received factor expression levels in responders and non-responders to individual received factor expression levels from the subject and wherein the machine learning algorithm outputs the resistance score.

According to some embodiments, the training set further comprises the sex of each responder and non-responder and the machine learning algorithm is applied to individual received factor expression levels from the subject and the subject's sex.

According to some embodiments, the method further comprises performing a dimensionality reduction step with respect to the plurality of factors, to reduce the number of factors in the plurality.

According to some embodiments, the dimensionality reduction step identifies a subset of principal factors and the training set comprises only expression levels of the subset of principal factors, optionally wherein the subset of principal factors are the factors that most evenly balance the predicted number of responders and non-responders.

According to some embodiments, the predetermined threshold is determined by performing a cross-validation within the training set.

According to some embodiments, the calculating comprises calculating a mean expression for each factor in responders and non-responders and wherein the resistance score is based on the ratio of deviation of the factor's expression in the subject from the calculated mean in responders to the deviation of the factor's expression in the subject from the calculated mean in non-responders.

According to some embodiments, the calculating further comprises calculating a distribution and standard deviation for each factor in responders and non-responders and wherein the deviation is measured as a multiple of the calculated standard deviation.

According to some embodiments, the resistance score is calculated by the equation monotonic

$$( \frac{\vert {Z}_{R} \vert}{\vert {z}_{N R} \vert + c} )$$

wherein Z_{R} is the deviation of the factors expression in the subject from the calculated mean in responders, Z_{NR} is the deviation of the factors expression in the subject from the calculated mean in non-responders, and c is a constant.

According to some embodiments, the predetermined threshold for the resistance score is about 2.9, and a resistance score above 2.9 indicates a factor is a resistance-associated factor.

According to some embodiments, the plurality of factors is at least 200 factors.

According to some embodiments, the predetermined number of resistance-associated factors is 3 and wherein a subject with more than 3 resistance-associated factors is predicted to be resistant to the therapy.

According to some embodiments, the receiving factors expression levels for a plurality of factors comprises:
a. receiving factors expression levels for a group of factors larger than the plurality in the population of responders and the population of non-responders;
b. for each factors of the group applying a machine learning algorithm trained on the received factors expression levels in responders and non-responders;
c. selecting a subgroup of factors for which the algorithm most evenly divided the subjects in the populations into responders and non-responders; and
d. designating the subgroup of factors as the plurality of factors.

According to some embodiments, the factors expression levels are factors expression levels in a biological sample provided by the subjects.

According to some embodiments, the biological sample is selected from blood plasma, whole blood, blood serum or peripheral blood mononuclear cells.

According to some embodiments, the biological sample is blood plasma.

According to some embodiments, the biological sample is provided by the subjects before receiving the therapy.

According to some embodiments, before is at most 24 hours before.

According to some embodiments, the biological sample is provided by the subject after receiving the therapy.

According to some embodiments, after receiving the therapy is after receiving a first treatment with the therapy.

According to some embodiments, after is at least 24 hours after.

According to some embodiments, the biological sample provided by each subject in a population is the same type of biological sample.

According to some embodiments, the biological samples provided by the responder population, the non-responder population and the subject are all the same type of biological sample.

According to some embodiments, the trained machine learning algorithm is trained by a method of the invention.

According to some embodiments, the disease is cancer.

According to some embodiments, the therapy is immune checkpoint inhibition, optionally wherein the immune checkpoint inhibition inhibits the PD-1/PD-L1 axis.

According to some embodiments, the at least one clinical parameter is selected from: a subject's age, sex, line of treatment, and expression of a biomarker within a sample from a subject.

According to some embodiments, the disease is cancer, the therapy is anti-PD-1 or anti-PD-L1 therapy and wherein expression of a target within a sample is expression of PD-L1 in a tumor sample.

According to some embodiments, the method further comprises at an inference stage, receiving as input, the number of resistance-associated factors expressed in a sample from a subject suffering from the disease and with unknown responsiveness to the therapy, and at least one clinical parameter of the subject with unknown responsiveness and applying the trained machine learning algorithm to the received input, to predict responsiveness to the therapy of the subject with unknown responsiveness.

According to some embodiments, the method further comprises administering the therapy or continuing to administer the therapy to the subject predicted to respond to the therapy.

According to some embodiments, the method further comprises discontinuing the therapy or not administering the therapy to the subject predicted to be resistant to the therapy.

According to some embodiments, the method further comprises administering an alternative therapy to the subject predicted to be resistant to the therapy.

According to some embodiments, the method further comprises administering to the subject predicted to be resistant to the therapy, the therapy in combination with an agent that modulates at least one of the resistance-associated factors or a factor in a functional pathway comprising the at least one resistance-associated factor, wherein:
a. the at least one resistance-associated factor is more highly expressed in the non-responders than the responders and increases activity of the pathway and the agent inhibits the resistance-associated factor or the pathway;
b. the at least one resistance-associated factor is more highly expressed in the non-responders than the responders and decreases activity of the pathway and the agent inhibits the resistance-associated factor or activates the pathway;
c. the at least one resistance-associated factor is more lowly expressed in the non-responders than the responders and increases activity of the pathway and the agent activates the resistance-associated factor or the pathway; or
d. the at least one resistance-associated factor is more lowly expressed in the non-responders than the responders and decreases activity of the pathway and the agent activates the resistance-associated factor or inhibits the pathway.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1C: Illustration of protein expression distributions in responders and non-responders populations at the single protein level. (1A-1C) Computer-generated examples of distributions of protein expression for responder and non-responder populations. Example of protein expression levels that may be considered as RAPs (light-gray dashed line) or are not RAPs (dark-gray dashed line) based on the population expression distribution data are shown.

FIG. 2: Illustration of the RAP score of Equation 2 as implemented in Algorithm 1. The RAP score was calculated using synthetic data, where the responder and non-responder populations were generated by sampling from a normal distribution. The synthetic populations expression levels are shown in histograms, the responder population in dark-grey and the non-responder population in light-grey. Given these distributions, the RAP score was calculated for each expression level. The resulting RAP score is plotted in a blue curve, the values are indicated in the secondary Y-axis on the right.

FIGS. 3A-3C: RAP score threshold determination based on AUC as a function of RAP score. AUC at each RAP score was calculated and the peak of the obtained curve was determined as the threshold (dotted line) for determining a certain protein as a RAP or not. (3A-3B) Graph describing determination of the RAP score threshold using the mathematical approach for protein measures at (3A) T1 and (3B) T0. (3C) Graph describing determination of the RAP score threshold using the machine learning approach.

FIGS. 4A-4B: (4A) Bar chart showing the number of RAPs for each patient in the test cohort (n=30). Responders—light-grey; non-responders—dark-grey. (4B) ROC curve for the RAP analysis.

FIG. 5: Heat map of hallmarks of cancer significantly enriched in six patients. The enrichment analysis was based on Fisher exact test (FDR<0.05). Next to each patient identifier the number of the patient's RAPs is indicated in brackets.

FIG. 6: Protein-protein network of the key RAPs in the current cohort. The network is based on STRING database. Each node (protein) is colored based on the hallmarks of cancer to which it is associated. A black circular frame indicates a targetable RAP. The size of each node correlates with the number of patients that had the examined RAP. The compartment/s of each node is indicated in the middle (based on Human Protein Atlas). I, intracellular. M, membranal. S, soluble. A protein can have more than one compartment.

FIG. 7: Chart of the significantly enriched hallmarks of cancer among the 19 RAPs. The analysis was done using Fisher exact test. Enrichment factor above 1 indicates enrichment.

FIG. 8: Heat map of the protein expression levels of the 19 RAPs in healthy tissues. The expression data is based on Human Protein Atlas (HPA) database.

FIG. 9: Heat map of the percentage of medium-high level staining in patients with different cancer types, including NSCLC. The expression data is based on Human Protein Atlas (HPA) database.

FIGS. 10A-10F: Clinical description of the 184 patients included in the analysis. (10A) Heatmap representing patient clinical characteristics: response to treatment (ORR1, ORR2, 1-year DCB); percent of cells expressing PD-L1 in biopsy immunostaining, a prognostic marker of response to treatment; treatment type: ICI only or combined treatment of ICI and chemotherapy; line of treatment: first line indicates ICI treatment was given as the first systemic treatment for NSCLC, advanced line indicates a previous non-ICI treatment was given before the current ICI treatment was administered. Sex indicates patient sex at birth. Histology indicates the lung cancer histological type (ADC-adenocarcinoma, SCC-squamous cell carcinoma). (10B-10C) Violin plots of the correlation of the patient age with response in each time point: (10B) ORR1 and (10C) ORR2. ORR1 and ORR2 are overall response determined 3 months and 6 months following treatment initiation, respectively. (10D-10E) Graphical display of the response groups in (10D) ORR1 and (10E) ORR2. NR=non-responders. R=responders (partial responders or complete responders). SD=stable disease (in the model they are included in the responder group). (10F) Graphical display of the division of the population into the development and the validation sets.

FIGS. 11A-11B: Performance of the classification model. ROC AUC was calculated using the final resistance score together with actual overall response evaluation at 3-month ORR, 6-month ORR and 1 year duration of clinical benefit (DCB) for both T0 and T1. Results at T0 for the (11A, upper panel) development set and for the (11A, lower panel and 11B, upper panel) validation set are shown. (11B, lower panel) A similar classification model was generated based on T1.

FIGS. 12A-12B: (12A) Patients sorted by their response probability score (calculated as 1-resistance score) based on protein levels at T0. Actual observed response at 3 months ORR is indicated by color for each patient. (12B) Dot plot of the agreement between the predicted response probability based on T0 protein expression and observed response probability at either 3 months, 6 months or 1 year. Each point on the graph indicates a specific patient, and the different time points are indicated by different hues and marker types. The black diagonal line indicates the line y=x, the red diagonal line indicates the fitted regression line for all the points and the goodness of fit of the regression (R²) is indicated. The horizontal lines indicate the average observed response probability for the 3 timepoints (color coded) across the entire validation set.

FIGS. 13A-13B: Survival analysis based on prediction results for ORR at 3-months based on T0 protein measurements for (13A) PFS and (13B) OS.

FIGS. 14A-14B: (14A) A functional network of all potential RAPs from this analysis. Each node represents a RAP, and the edge between nodes indicates a functional relation. Nodes with a larger size, and protein name provided, indicate investigational new drugs (INDs) in combination with immunotherapy. The nodes are colored based on the protein function. (14B) Functional network for two patients a predicted non-responder (upper) and a predicted responder (lower). RAPs detected for each patient are outlined in black. The non-responder patient had 44 RAPs detected and the responder had 10 RAPs detected.

FIG. 15: Functional differences between RAPs higher in each response group. Each polygon in the Voronoi plot represents a RAP, and the size correlates with the difference between responders and non-responders. While non-responder RAPs are involved in splicing, signaling and cytoskeleton-related processes, the responder RAPs mainly involved proteolysis and cell adhesion. Each color indicates a different overall function.

FIG. 16: A table describing the clinical parameters of the 339 patients included in the analysis.

FIG. 17: Line graphs of patient number at each time point are indicated per response group (NR, non-responder; R, responder) and in total. The patient cohort was divided into development and validation sets.

FIGS. 18A-18B: Performance of clinical parameter-based predictive models. (18A) Receiver operating characteristics (ROC) plot of the PD-L1-based predictive model. (18B) ROC plot of the predictive model based on PD-L1, age and treatment line. The area under the curve (AUC) values are indicated for each time point.

FIGS. 19A-19B: (19A) Development of the RAP-based predictive model. A cohort of stage IV NSCLC patients receiving ICB-based therapy was assembled. Pre-treatment blood samples were obtained, and plasma proteomes were profiled. Clinical response to treatment was assessed at 3, 6 and 12 months after starting treatment, while patient follow-up continued for up to 2 years. A predictive model for ICB response was developed for each response assessment time point as follows: Proteins displaying differential plasma levels in responders and non-responders (collectively termed Response Associated Proteins; RAPs) were selected for model training using a statistical test. A predictive model for response was developed per RAP using machine learning algorithm. Response predictions inferred from each RAP were summed up to yield a RAP score per patient. RAP scores were linearly scaled to values between 0 and 1, enabling the conversion of a given patient's RAP score into a response probability. (19B) Development and validation of the RAP-based model. The cohort was divided into development and validation sets (75% and 25%, respectively). The development set was further randomly divided into train and test sets (75% and 25%, respectively). The train set was used for RAP selection followed by model training resulting in a predictive model per RAP. Response predictions were then generated per RAP for each patient in the test set. Response predictions from all selected RAPs were summed up to yield a RAP score per patient in the test set. The process was repeated 80 times, each time with a random division of development set patients into train and test sets. The RAP scores were averaged per patient in the development set and then linearly scaled, enabling the conversion of a given patient's RAP score into a response probability (a value between 0 and 1). The model was then locked and tested in the independent validation set.

FIGS. 20A-20E: RAP identification during model development. (20A) Histograms showing the number of identified RAPs grouped according to the number of times they were selected over 80 iterations. The top, middle and bottom histograms are for the 3-, 6- and 12-month time points, respectively. (20B). The total number of RAPs identified per time point. Some proteins quantified by the method is redundant. The numbers of overall and non-redundant RAPs are indicated by light grey and medium gray bars, respectively). The numbers of non-redundant RAPs identified at least 40 times in a total of 80 iterations are indicated by dark grey bars. (20C) A Venn diagram showing the number of RAPs identified per time point. (20D) Hierarchical clustering based heatmap showing the number of iterations in which a given protein was classified as a RAP. (20E) Voronoi plots displaying the main biological functions of the RAPs per time point. Each polygon represents a RAP, and the size correlates with the number of times that the protein was selected as a RAP.

FIG. 21: Performance of the RAP-based predictive model. A Waterfall plot showing predicted response probabilities sorted from lowest to highest. Observed responders and non-responders are shown as light and dark grey bars, respectively.

FIGS. 22A-22D: Comparison between response probabilities at sequential time points. Each dot represents a patient in the cohort. Response probability at one time point is plotted against response probability at the subsequent time point. The colors indicate patient response labels per time point, and whether the response label changed between time points. R, responder; NR, non-responder. (22A). Comparison between 3- and 6-months. (22B). Comparison between 3 and 12 months. (22C). Comparison between 6 and 12 months. (22D). Sankey plot displaying the flow of response labeling over time. R, responder; NR, non-responder; NA, not available.

FIG. 23: Enrichment analysis for response probabilities and observed rates at each time point. The enrichment analysis was done using 2D-enrichment test. The X-axis indicates the enrichment factor for predicted response probability. The Y-axis indicates the enrichment factor for observed rates (as defined by the fraction of responders within a range of ±0.15 response probability). Positive and negative values indicate enrichment in high or low response probabilities or observed rates, respectively. The solid line indicates the Y=X line.

FIGS. 24A-24B: (24A) Overall survival analysis of patients stratified to high and low response probability groups. The median response probability per time point was used as the stratification threshold. (24B) Progression-free survival analysis of patients stratified to high and low response probability groups. The median response probability per time point was used as the stratification threshold. HR, hazard ratio. CI, confidence interval.

FIGS. 25A-25B: (25A) Predicted response probability as a function of observed response rate. Each dot represents a patient. The observed response rate for each predicted response probability datapoint refers to the proportion of observed responders within a patient group assigned the response probability ±0.15. Y=X is indicated by a black line. The goodness of fit is indicated. (25B) Receiver operating characteristics (ROC) plot for the RAP-based model per time point. The area under the curve (AUC) is indicated. The red dashed line indicates AUC=0.5.

FIGS. 26A-26B: The RAP-based model outperforms clinical parameter-based models. Predictive performance was compared across five models: RAP-based model (RAP); PD-L1-based model (PD-L1); Clinical model (CM); Integrated RAP+PD-L1; Integrated RAP+CM. (26A) ROC curves plots of the five models at each time point. CM, clinical model. The dashed line indicates AUC-0.5. (26B) Forest plot comparing the five models. Top, Cox regression analysis based on overall survival (OS) data. Bottom, Cox regression analysis based on progression-free survival (PFS) data.

FIGS. 27A-27D: Performance of the RAP-based clinical predictive model at 3 months (27A), 6 months (27B) and 1 year (27C). (27D) Predicted response probability as a function of observed response rate. Each dot represents a patient. The observed response rate for each predicted response probability datapoint refers to the proportion of observed responders within a patient group assigned the response probability ±0.15. Y=X is indicated by a black line. The goodness of fit is indicated.

FIG. 28: Performance of RAP-based model for different patient subsets.

FIGS. 29A-29B: The RAP-based model identifies a PD-L1 high subset of patients that may benefit from combination therapy. (29A) Kaplan Meier plots for the three PD-L1 groups. Left, OS; right, PFS. (29B) Overall survival analysis of PD-L1-high and PD-L1 low-neg subgroups under ICB (part I) or ICB-chemotherapy (part II) treatment modalities.

FIGS. 30A-30B: Progression free survival analysis of PD-L1-high and PD-L1 low-neg subgroups under ICB (30A) or ICB-chemotherapy (30B) treatment modalities.

### DETAILED DESCRIPTION OF THE INVENTION

The present invention, in some embodiments, provides methods of predicting response of a subject to a therapy.

By a first aspect, there is provided a method of predicting response of a subject to a therapy, the method comprising:
a. receiving expression levels for a plurality of factors
   i. in a population of subjects known to respond to the therapy (responders);
   ii. in a population of subjects known to not respond to the therapy (non-responders); and
   iii. in the subject;
b. calculate for at least one factor of the plurality of factors a resistance score; and
c. classify a factor with a resistance score beyond a threshold as a resistance-associated factor;
   wherein a subject with a number of resistance-associated factors beyond a predetermined number is predicted to be resistant to the therapy, thereby predicting the response of a subject to a therapy.

By another aspect, there is provided a method of predicting response of a subject to a therapy, the method comprising:
a. receiving expression levels for a plurality of factors
   i. in a population of subjects known to respond to the therapy (responders);
   ii. in a population of subjects known to not respond to the therapy (non-responders); and
   iii. in the subject;
b. calculate for at least one factor of the plurality of factors a resistance score;
c. classify a factor with a resistance score beyond a threshold as a resistance-associated factor;
d. sum the number resistance-associated factors; and
e. apply a trained machine learning algorithm to the number of resistance-associated factors and at least one clinical parameter, wherein the trained machine learning algorithm outputs a final resistance score and a final resistance score beyond a predetermined threshold indicates the subject is resistant to the therapy;
thereby predicting the response of a subject to a therapy.

By another aspect, there is provided a method of predicting response of a subject to a therapy, the method comprising:
a. receiving expression levels for a plurality of factors
   i. in a population of subjects known to respond to the therapy (responders);
   ii. in a population of subjects known to not respond to the therapy (non-responders); and
   iii. in the subject;
b. calculate for factors of the plurality of factors a resistance score, wherein the resistance score is based on the similarity of the factor expression level in the subject to the factor expression level in the responders and the similarity to the factor expression level in the subject to the factor expression level in the non-responders and wherein the calculating comprises applying a trained machine learning algorithm that outputs the resistance score; and
c. sum the calculated resistance scores to produce a total resistance score;
   wherein a subject with a total resistance score beyond a predetermined threshold is predicted to be resistant to said therapy;
   thereby predicting the response of a subject to a therapy.

By another aspect, there is provided a method comprising:
at a training stage, training a machine learning algorithm on a training set comprising:
(i) a number of resistance-associated factors expressed in samples from subjects suffering from a disease and known to be responsive to a therapy and from subjects suffering from the disease and known to be non-responsive to the therapy;
(ii) at least one clinical parameter of the subjects; and
(iii) labels associated with the responsiveness of the subjects;
to produce a trained machine learning algorithm

By another aspect, there is provided a method comprising:
at a training stage, training a machine learning algorithm on a training set comprising:
(i) factor expression levels of resistance-associated factors in samples from subjects suffering from a disease and known to be responsive to a therapy and from subjects suffering from the disease and known to be non-responsive to the therapy;
(ii) at least one clinical parameter of the subjects; and
(iii) labels associated with the responsiveness of the subjects;
to produce a trained machine learning algorithm.

In some embodiments, the method is a diagnostic method. In some embodiments, the method is an in vitro method. In some embodiments, the method is an ex vivo method. In some embodiments, the method is a computer implemented method. In some embodiments, the method is a statistical method. In some embodiments, the method is a method that cannot be performed in a human mind. In some embodiments, the method is a computerized method. In some embodiments, the processor is a computer processor. In some embodiments, the processor is a computer.

In some embodiments, the method is for predicting response to therapy. In some embodiments, the method is for determining response to therapy. In some embodiments, the method is for determining response score. In some embodiments, the method is for determining response probability. In some embodiments, a response probability is a response score. According to some embodiments, resistance score is determined. According to other embodiments, prediction of resistance probability is determined. According to some other embodiments, resistance probability below 20% indicates the subject is responsive to therapy. According to some embodiments, response score is determined. According to other embodiments, prediction of response probability is determined. According to some other embodiments, response probability beyond 80% indicates the subject is responsive to therapy. In some embodiments, beyond is above. In some embodiments, beyond is below. It will be understood by a skilled artisan that a scale can be designed to be measured in either direction and so above/below depends on the construction of the scale.

In some embodiments, the method is for determining if a subject is a responder to the therapy. In some embodiments, the method is for determining if a subject is a non-responder to the therapy. In some embodiments, the method is for predicting a subject's response to therapy. In some embodiments, the method is for monitoring response to the therapy. In some embodiments, the method is for determining if the therapy should continue or be adjusted (e.g., by further treating the subject with an additional therapy including but not limited to an agent determined by the RAP analysis provided hereinbelow). In some embodiments, the method is for determining a subject as being a responder to the therapy, or a non-responder to the therapy. In some embodiments, the method is for determining a subject as being a responder to the therapy, a non-responder to the therapy, or as having a stable diseased state. In some embodiments, the method is for predicting if a subject will respond to the therapy, or not respond to the therapy.

In some embodiments, non-response comprises progressive disease. In some embodiments, non-response comprises cancer progression. In some embodiments, non-response comprises stable disease. In some embodiments, non-response comprises a worsening of symptoms of the disease. In some embodiments, non-response is not the development of side effects. In some embodiments, non-response comprises growth, metastasis and/or continued proliferation of a cancer. In some embodiments, response is stable disease. In some embodiments, response comprises remission. In some embodiments, remission is minimal remission. In some embodiments, remission is partial remission. In some embodiments, remission is complete remission. In some embodiments, response is measured using the overall response rate (ORR). A trained physician will be familiar with methods of determining response and specifically the ORR. In some embodiments, response is measured using Response Evaluation Criteria In Solid Tumors (RECIST). In some embodiments, response comprises survival. In some embodiments, survival is overall survival. In some embodiments, survival is progression free survival. In some embodiments, response comprises a durable clinical benefit (DCB).

In some embodiments, the subject is a mammal. In some embodiments, the subject is a human. In some embodiments, the subject suffers from a disease. In some embodiments, the disease is treatable by the therapy. In some embodiments, the disease is cancer. In some embodiments, the disease is treatable by an immune checkpoint inhibitor (ICI). In some embodiments, the cancer is a PD-L1 positive cancer. In some embodiments, the cancer is a PD-L1 negative cancer. In some embodiments, the cancer is solid cancer. In some embodiments, the cancer is a tumor. In some embodiments, the cancer is selected from hepato-biliary cancer, cervical cancer, urogenital cancer (e.g., urothelial cancer), testicular cancer, prostate cancer, thyroid cancer, ovarian cancer, nervous system cancer, ocular cancer, lung cancer, soft tissue cancer, bone cancer, pancreatic cancer, bladder cancer, skin cancer, intestinal cancer, hepatic cancer, rectal cancer, colorectal cancer, esophageal cancer, gastric cancer, gastroesophageal cancer, breast cancer (e.g., triple negative breast cancer), renal cancer (e.g., renal carcinoma), skin cancer, head and neck cancer, leukemia and lymphoma. In some embodiments, the cancer is selected from skin cancer, and lung cancer. In some embodiments, the cancer is skin cancer. In some embodiments, the cancer is lung cancer. In some embodiments, the skin cancer is melanoma. In some embodiments, the lung cancer is small cell lung cancer. In some embodiments, the lung cancer is non-small cell lung cancer. In some embodiments, the subject is naïve to therapy before the first determining. In some embodiments, the subject has not received the therapy before the first determining. In some embodiments, the subject has received the therapy previously. In some embodiments, the subject has previously been treated by a therapy other than the therapy. In some embodiments, the subject is naïve to any therapy. In some embodiments, the subject is naïve to immunotherapy. In some embodiments, the therapy is the first line of treatment. In some embodiments, the therapy is an advanced line of treatment.

In some embodiments, the therapy is an anticancer therapy. In some embodiments, the anticancer therapy is radiation. In some embodiments, the anticancer therapy is chemotherapy. In some embodiments, the therapy is immunotherapy. In some embodiments, the anticancer therapy is immunotherapy. In some embodiments, the anticancer therapy is targeted therapy. In some embodiments, the anticancer therapy is selected from radiation, chemotherapy, immunotherapy, targeted therapy, hormonal therapy, anti-angiogenic therapy and photodynamic therapy, thermotherapy, surgery, and a combination thereof. In some embodiments, the immunotherapy is selected from immune checkpoint inhibition, immune checkpoint modulation, immune checkpoint blockade, adoptive-cell transfer therapy, oncolytic virus therapy, vaccine therapy, immune system modulation and therapy using monoclonal antibodies. In some embodiments, an immunotherapy is selected from immune checkpoint inhibitors, immune checkpoint modulators, immune checkpoint blockers, adoptive-cell transfer therapy, oncolytic virus therapy, treatment vaccines, immune system modulators and monoclonal antibodies. In some embodiments, the immunotherapy is an immune checkpoint inhibitor. In some embodiments, the immunotherapy is immune checkpoint blockade. In some embodiments, an immunotherapy is administered in combination with one or more conventional cancer therapy including chemotherapy, targeted therapy, steroids, and radiotherapy. Combinations of ICI and chemotherapy/radiotherapy/targeted therapy have been studied in multiple clinical trials. It will be understood by a skilled artisan that the predictive proteins disclosed herein are predictive in immunotherapy as a monotherapy, as well as part of a combination therapy.

In some embodiments, the immunotherapy is a plurality of immunotherapies. In some embodiments, the immunotherapy is immune checkpoint blockade. In some embodiments, the immunotherapy is immune checkpoint protein inhibition. In some embodiments, the immunotherapy is immune checkpoint protein modulation. In some embodiments, the immunotherapy comprises immune checkpoint inhibition. In some embodiments, the immunotherapy comprises immune checkpoint modulation. In some embodiments, immune checkpoint blockade and/or immune checkpoint inhibition comprises administering to the subject an immune checkpoint inhibitor. In some embodiments, inhibition comprises administering an immune checkpoint inhibitor. In some embodiments, the inhibitor is a blocking antibody. In some embodiments, the immunotherapy comprises immune checkpoint blockade. In some embodiments, modulation comprises administering an immune checkpoint modulator. In some embodiments, immune checkpoint modulation comprises administering to the subject an immune checkpoint modulator.

As used herein, the term “an immune checkpoint inhibitor (ICI)” refers to a single ICI, a combination of ICIs and a combination of an ICI with another cancer therapy. The ICI may be a monoclonal antibody, a dual-specific antibody, a humanized antibody, a fully human antibody, a fusion protein, or a combination thereof directed to blocking, inhibition or modulation of immune checkpoint proteins. In some embodiments, an immune checkpoint inhibitor is an immune checkpoint modulator. In some embodiments, an immune checkpoint inhibitor is an immune checkpoint blocker. In some embodiments, the immune checkpoint protein is selected from PD-1 (Programmed Death-1); PD-L1; PD-L2; CTLA-4 (Cytotoxic T-Lymphocyte-Associated protein 4); A2AR (Adenosine A2A receptor), also known as ADORA2A; B7-H3, also called CD276; B7-H4, also called VTCN1; B7-H5; BTLA (B and T Lymphocyte Attenuator), also called CD272; IDO (Indoleamine 2,3-dioxygenase); KIR (Killer-cell Immunoglobulin-like Receptor); LAG-3 (Lymphocyte Activation Gene-3); TDO (Tryptophan 2,3-dioxygenase); TIM-3 (T-cell Immunoglobulin domain and Mucin domain 3); VISTA (V-domain Ig suppressor of T cell activation); NOX2 (nicotinamide adenine dinucleotide phosphate NADPH oxidase isoform 2); SIGLEC7 (Sialic acid-binding immunoglobulin-type lectin 7), also called CD328; SIGLEC9 (Sialic acid-binding immunoglobulin-type lectin 9), also called CD329; OX40 (Tumor necrosis factor receptor superfamily, member 4) also called CD134; and TIGIT. In some embodiments, the immune checkpoint protein is selected from PD-1, PD-L1 and PD-L2. In some embodiments, the immune checkpoint protein is selected from PD-1 and PD-L1. In some embodiments, the immune checkpoint protein is CTLA-4. In some embodiments, the immune checkpoint protein is PD-1. In some embodiments, immune checkpoint blockade comprises an anti-PD-1/PD-L1/PD-L2 immunotherapy. In some embodiments, immune checkpoint blockade comprises an anti-PD-1 immunotherapy. In some embodiments, immune checkpoint blockade comprises an anti-PD-1 and/or anti-PD-L1 immunotherapy. In some embodiments, immune checkpoint blockade comprises an anti CTLA-4 immunotherapy. In some embodiments, immune checkpoint blockade comprises an anti-PD-1 and/or anti-PD-L1 immunotherapy and an anti CTLA-4 immunotherapy.

In some embodiments, the resistance-associated factor is determined by a method comprising:
a. receiving expression levels for a plurality of factors
   i. in a population of subjects known to respond to the therapy (responders);
   ii. in a population of subjects known to not respond to the therapy (non-responders); and
   iii. in the subject;
b. calculate for at least one factor of the plurality of factors a resistance score; and
c. classify a factor with a resistance score beyond a threshold as a resistance-associated factor.

In some embodiments, resistance-associated factors are in each subject. In some embodiments, resistance-associated factors are in the responders. In some embodiments, resistance-associated factors are in the non-responders. In some embodiments, the resistance-associated factors are labeled with the labels. In some embodiments, the resistance-associated factors are resistance-associated proteins.

In some embodiments, the immunotherapy is a blocking antibody. In some embodiments, the immunotherapy is administration of a blocking antibody to the subject.

In some embodiments, the ICI is a monoclonal antibody (mAb) against PD-1 or PD-L1. In some embodiments, the ICI is a mAb that neutralizes/blocks/inhibits/modulates the PD-1 pathway. In some embodiments, the ICI is a mAb against PD-1. In some embodiments, the anti-PD-1 mAb is Pembrolizumab (Keytruda; formerly called lambrolizumab). In some embodiments, the anti-PD-1 mAb is Nivolumab (Opdivo). In some embodiments, the anti-PD-1 mAb is Pidilizumab (CT0011). In some embodiments, the anti-PD-1 mAb is Cemiplimab (Libtayo, REGN2810). In some embodiments, the anti-PD-1 mAb is any one of AMP-224, MEDI0680, or PDR001. In some embodiments, the ICI is a mAb against PD-L1. In some embodiments, the anti-PD-L1 mAb is selected from Atezolizumab (Tecentriq), Avelumab (Bavencio), and Durvalumab (Imfinzi). In some embodiments, the anti-PD-L1 mAb is Atezolizumab. In some embodiments, the anti-PD-L1 mAb is Durvalumab. In some embodiments, the ICI is a mAb against CTLA-4. In some embodiments, the anti-CTLA-4 mAb is ipilimumab.

As used herein, the term “factor” refers to any measurable biological molecule produced by the subject. In some embodiments, the factor is a protein. In some embodiments, the factor is an RNA. In some embodiments, the factor is a gene. In some embodiments, the factor is a secreted factor. In some embodiments, the secreted factors are selected from cytokines, chemokines, growth factors, soluble receptors and enzymes. In some embodiments, the factor is a soluble factor. In some embodiment, the factor is cellular factor. In some embodiments, the factor is membranal factor. In some embodiments, the factor is a cell adhesion molecule. In some embodiments, the factor is a factor found in blood. In some embodiments, the factor is a host-generated factor. In some embodiments, the factor is a resistance factor.

In some embodiments, the expression is protein expression. In some embodiments, the expression is secreted protein expression. In some embodiments, protein expression is soluble protein expression. In some embodiment, the expression is cellular protein expression. In some embodiments, the expression is membranal protein expression. In some embodiments, the expression is mRNA expression. In some embodiments, the expression is protein expression or mRNA expression. In some embodiments, expression level is concentration. In some embodiments, concentration is concentration level. It will be understood by a skilled artisan that when the presence of factor is measured in a liquid sample the expression can be provided as a concentration such as mg/ml or in arbitrary units according to the method of determining the factor's expression. Arbitrary units can be selected from relative fluorescence unit (RFU) and Normalized Protein expression (NPX), or any other arbitrary units used as measurement of expression. The terms “expression” and “expression levels” are used herein interchangeably and refer to the amount of a gene product present in the sample. In some embodiments, gene product includes polynucleotide, e.g., tumor DNA, circulating tumor DNA, or circulating DNA. In some embodiments, the DNA is cell-free DNA. In some embodiments, determining comprises quantification of expression levels. In some embodiments, determining comprises normalization of expression levels. Determining of the expression level of the factor can be performed by any method known in the art. Methods of determining protein expression include, for example, antibody arrays, immunoblotting, immunohistochemistry, flow cytometry (FACS), ELISA, proximity extension assay (PEA), aptamer-based assays, proteomics arrays, proteome sequencing, flow cytometry (CyTOF), multiplex assays, mass spectrometry and chromatography. In some embodiments, determining protein expression levels comprises ELISA. In some embodiments, determining protein expression levels comprises protein array hybridization. In some embodiments, determining protein expression levels comprises mass-spectrometry quantification. In some embodiments, determining protein expression levels comprises PEA. In some embodiments, determining protein expression levels comprises aptamers. Methods of determining mRNA expression include, for example, RT-PCR, quantitative PCR, real-time PCR, microarrays, northern blotting, in situ hybridization, next generation sequencing, and massively parallel sequencing.

In some embodiments, the receiving factor expression levels is providing factor expression levels. In some embodiments, the receiving factor expression levels is determining factor expression levels. In some embodiments, determining is measuring. In some embodiments, the measuring is in a sample. In some embodiments, the expression levels were detected in a sample. In some embodiments, the sample is a biological sample.

In some embodiments, the sample is provided by the subjects. In some embodiments, the sample is provided by the subject. In some embodiments, the sample is provided by a responder. In some embodiments, the sample is provided by a non-responder. In some embodiments, each subject of the population of responders provided a sample. In some embodiments, each subject of the population of non-responders provided a sample. In some embodiments, the sample is provided by a subject before receiving the therapy. In some embodiments, the sample is provided by a subject after receiving the therapy. In some embodiments, the determining is directly in the sample. In some embodiments, the determining is in the unprocessed sample. In some embodiments, the determining is in a processed sample. In some embodiments, the method further comprises processing the sample. In some embodiments, processing comprises isolating proteins from the sample. In some embodiments, processing comprises isolating nucleic acids from the sample. In some embodiments, the nucleic acid is RNA. In some embodiments, the RNA is mRNA. In some embodiments, the processing comprises lysing cells in the sample.

As used herein, the terms “peptide”, “polypeptide” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

In some embodiments, the sample is a biological sample. In some embodiments, the sample is tissue. In some embodiments, the sample is a fluid. In some embodiments, the fluid is a biological fluid. In some embodiments, the sample is from the subject. In some embodiments, the sample is not a tumor sample. In some embodiments, the sample is a tumor sample. In some embodiments, the sample is not a hematopoietic cancer and the sample is a blood sample. In some embodiments, the sample is a sample that does not comprise cancer cells. In some embodiments, a blood sample comprises a peripheral blood sample, serum sample and a plasma sample. In some embodiments, the sample is a plasma sample. In some embodiments, the sample is a serum sample. In some embodiments, processing comprises isolating plasma. In some embodiments, processing comprises isolating serum. In some embodiments, the biological fluid is selected from, blood, plasma, serum, lymph, cerebral spinal fluid, urine, feces, semen, tumor fluid and gastric fluid. In some embodiments, the sample obtained from the subject and the responders are the same type of sample. In some embodiments, the sample obtained from the subject and the responders are different types of samples. In some embodiments, the sample obtained from the subject and the non-responders are the same type of sample. In some embodiments, the sample obtained from the subject and the non-responders are different types of samples. In some embodiments, the sample obtained from the non-responders and the responders are the same type of sample. In some embodiments, the sample obtained from the non-responders and the responders are different types of samples. In some embodiments, the sample obtained from the subject, the non-responders and the responders are the same type of sample. In some embodiments, the sample obtained from the subject, the non-responders and the responders are blood samples. In some embodiments, the sample obtained from the subject, the non-responders and the responders are plasma samples. In some embodiments, the sample obtained from the subject, the non-responders and the responders are serum samples In some embodiments, the sample obtained from the subject, the non-responders and the responders are different types of samples.

In some embodiments, a factor is a factor of the plurality of factors. In some embodiments, expression levels of a plurality of factors are received. In some embodiments, expression levels of at least 2, 3, 4, 5, 6,7, 8, 9, 10, 11, 12, 13, 14, 15, 16, 17, 18, 19, 20, 21, 22, 23, 24, 25, 30, 35, 40, 45, 50, 100, 150, 200, 250, 300, 400, 500, 600, 700, 800, 900, 1000, 1100, 1200, 1300, 1400, 1500, 1600, 1700, 1800, 1900, 2000, 3000, 4000, 5000, 6000, 7000, 8000, 9000, 10000, 12000, 15000, 20000, 25000, 30000, 35000, or 40000 factors is received. Each possibility represents a separate embodiment of the invention. In some embodiments, a plurality is at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 100, 150, 200, 250, 300, 400, 500, 600, 700, 800, 900, 1000, 1100, 1200, 1300, 1400, 1500, 1600, 1700, 1800, 1900, 2000, 3000, 4000, 5000, 6000, 7000, 8000, 9000, 10000, 12000, 15000, 20000, 25000, 30000, 35000, or 40000. Each possibility represents a separate embodiment of the invention. In some embodiments, expression levels of at least 200 factors are received. In some embodiments, expression levels of at least 400 factors are received. In some embodiments, expression levels of at least 1000 factor are received. In some embodiments, expression levels of at least 5000 factors are received. In some embodiments, expression levels of at least 6000 factors are received. In some embodiments, expression levels of at least 7000 factors are received. In some embodiments, expression levels of at least 8000 factors are received.

In some embodiments, the population of responders suffers from the disease. In some embodiments, the responders all have the same disease. In some embodiments, the population of non-responders suffers from the disease. In some embodiments, the non-responders all suffer from the same disease. In some embodiments, the population of responders and non-responders all suffer from the same disease. In some embodiments, the population of responders and the subject suffer from the same disease. In some embodiments, the population of non-responders and the subject suffer from the same disease. In some embodiments, the population of non-responders, the population of responders and the subject suffer from the same disease.

In some embodiments, the expression levels are from the subject before receiving the therapy. In some embodiments, the expression levels are determined for the subject before receiving the therapy. In some embodiments, the expression levels are from time T0. In some embodiments, the sample is provided by the subject before receiving the therapy. In some embodiments, the expression levels are from the subject before receiving a first treatment of the therapy. In some embodiments, a treatment is a dose. In some embodiments, a treatment is a regimen.

In some embodiments, before is at least 1 hour, 2 hours, 3 hours, 6 hours, 8 hours, 12 hours, 1 day, 2 days, 3 days, 5 days, 1 week, 2 weeks, 3 weeks, 4 weeks, 1 month, 2 months, 3 months, 4 months, 5 months, or 6 months before the therapy or before administration of the therapy. Each possibility represents a separate embodiment of the invention. In some embodiments, before is at least 1 hour before. In some embodiments, before is just before the therapy or before administration of the therapy. In some embodiments, before is at most 1 hour, 2 hours, 3 hours, 4 hours, 6 hours, 9 hours, 12 hours, 18 hours, 24 hours, 2 days, 3 days, 5 days, 1 week, 2 weeks, 3 weeks, 4 weeks, 1 month, 2 months, 3 months, 4 months, 5 months, or 6 months before the therapy or before administration of the therapy. Each possibility represents a separate embodiment of the invention. In some embodiments, before is at most 24 hours before the therapy or before administration of the therapy. In some embodiments, administration of the therapy is the first administration of the therapy. In some embodiments, administration of the therapy is any administration of the therapy.

In some embodiments, the expression levels are from the subject after receiving the therapy. In some embodiments, the expression levels are from time T1. In some embodiments, the sample is provided by the subject after receiving the therapy. In some embodiments, the expression levels are from the subject after receiving a first treatment of the therapy. In some embodiments, the expression levels are from the subject after receiving any treatment with the therapy.

In some embodiments, after is at a time after initiation of the therapy, or after administration of the therapy, sufficient for altered expression of the at least one factor. In some embodiments, after is at a time after initiation of the therapy, or after administration of the first treatment of the therapy. In some embodiments, after is at least 1 day, 2 days, 3 days, 4 days, 5 days, 6 days, 1 week, 2 weeks, 3 weeks, 4 weeks, 6 weeks, 1 month, 2 months, 3 months, 4 months, 5 months, 6 months, or a year after. Each possibility represents a separate embodiment of the invention. In some embodiments, after is at least 24 hours after. In some embodiments, after is at least 2 weeks after. In some embodiments, after is at least 3 weeks after. In some embodiments, after is at least 6 weeks after. In some embodiments, after is at most 1 week, 2 weeks, 3 weeks, 4 weeks, 6 weeks, 1 month, 2 months, 3 months, 4 months, 5 months, 6 months or a year after initiation of the therapy, or after administration of the therapy. Each possibility represents a separate embodiment of the invention.

In some embodiments, the receiving expression levels comprises receiving factor expression levels for a group of factors larger than the plurality of factors. In some embodiments, the received expression levels for the larger group are received for responders and non-responders. In some embodiments, a subgroup of proteins is selected from the group. In some embodiments, the subgroup is designated the plurality of factors. In some embodiments, the method comprises designating. In some embodiments, the receiving further comprises for each factor of the group applying a machine learning algorithm. In some embodiments, the algorithm classifies factors as from responders and non-responders. In some embodiments, the algorithm outputs if a subject that provided the sample that had the measured factor expression level is a responder or non-responder. In some embodiments, the receiving further comprises selecting a subgroup of factors for which the algorithm most evenly divides the subjects into responders and non-responders. In some embodiments, the subjects are all the subjects in the populations of responders and non-responders. In some embodiments, the factors are processed with an algorithm that most evenly divides all subjects, responders and non-responders, into groups of responders and non-responders (even if designations are incorrect) are selected as the subgroup. In some embodiments, the algorithm is trained on the received factor expression levels in responders and non-responders. In some embodiments, the algorithm is trained on a training set. In some embodiments, training is on expression levels and tags indicating if an expression level was from a responder or non-responder. In some embodiments, training is on expression levels, clinical information and tags indicating if an expression level was from a responder or non-responder.

In some embodiments, the subgroup comprises the factors whose algorithm most evenly divides the subjects. In some embodiments, evenly divides is into responders and non-responders. In some embodiments, the subgroup is the top 50, 100, 150, 200, 250, 300, 350, 400, 450, 500, 600, 700, 750, 800, 900, 1000, 1100, 1200, 1300, 1400, 1500, 1600, 1700, 1800, 1900, 2000, 3000, 4000, or 5000. Each possibility represents a separate embodiment of the invention. In some embodiments, the subgroup is the top 50. In some embodiments, the subgroup is the top 100. In some embodiments, the subgroup is the top 200. In some embodiments, the subgroup is the top 500.

In some embodiments, the method further comprises performing a dimensionality reduction step. In some embodiments, the reduction is with respect to the plurality of factors. In some embodiments, the reduction is reducing the number of factors in the plurality. In some embodiments, the dimensionality reduction step identifies a subgroup or a subset of factors. In some embodiments, factors are principal factors. In some embodiments, the training set comprises only the expression levels of the subset/subgroup of factors. In some embodiments, the subgroup or subset of factors are the factors that most evenly balance the predicted number of responders and non-responders. In some embodiments, predicted is predicted by the machine learning algorithm. In some embodiments, the machine learning algorithm is the trained machine learning algorithm. In some embodiments, the machine learning algorithm is the machine learning algorithm during training.

In some embodiments, a preprocessing stage may take place to preprocess the received expression levels. In some embodiments, the preprocessing stage may comprise at least one of data cleaning and normalizing, feature selection, feature extraction, dimensionality reduction, and/or any other suitable preprocessing method or technique. Feature selection can be performed by statistical tests, such as the Kolmogorov Smirnov (KS) test, or any other test known in the art.

In some embodiments, factor selection and/or dimensionality reduction steps may be performed, to reduce the number of factors in each sample and/or to obtain a set of principal factors, e.g., those factors that may have significant predictive power. Accordingly, in some embodiments, a factor selection and/or dimensionality reduction step may result in a reduction of the number of factors in each sample and/or set of values. In some embodiments, dimensionality reduction selects principal factors, e.g., proteins, based on the level of response predictive power a factor generates with respect to the desired prediction. In specific embodiments, the dimensionality reduction involves regarding all or some factors as vector components and calculating their norm.

In some embodiments, any suitable factor selection and/or dimensionality reduction method or technique may be employed, such as, but not limited to:
ANOVA with S₀ parameter: Analysis of variance with an additional parameter (S₀) that controls for the relative importance of features based on resulted test p-values and difference between the group means (see, e.g., Tusher, Tibshirani and Chu, PNAS 98, pp 5116-21, 2001).
Scalable EMpirical Bayes Model Selection (SEMMS): An empirical Bayes feature selection method which applies a parsimonious mixture model to identify significant predictors (see, e.g., Bar, Booth, and Wells. A scalable empirical Bayes approach to variable selection in generalized linear models, 2019).
L2N: A method for differential expression analysis that uses a three-component mixture model. The model consists of two log-normal components (L2) for differentially expressed features, one component for under-expressed features and the other for overexpressed features, and a single normal component (N) for non-differentially expressed features (see, e.g., Bar and Schifano. Differential variation and expression analysis. Stat 8, e237, doi: 10.1002/sta4.237, 2019).
Genetic algorithms: A family of heuristic optimization algorithms that employ organic evolutionary techniques such as random mutations, recombination, and natural selection as methods for achieving optimal configurations (see, e.g., Popovic, Sifrim, Pavlopoulos, Moreau, and Bart De Moor. A Simple Genetic Algorithm for Biomarker Mining. 2012).
Naïve classifier: The naïve classifier evaluates a response score by reducing the dimension to a single score. This is performed by regarding all features (e.g., specific profiles such as protein expression levels) as component of a vector and calculating its norm. The dimension reduction reduces the possible risk of an over-fitting. In some embodiments, the vector components are normalized according to the typical component value among patients that belong to the same response group (e.g., responders), such that the normalized norm quantifies the amount of deviation from the typical respective class value. In additional embodiments, the naïve classifier enables training using data of subjects that belong only to part of the response groups.

As used herein, the term “responder” or a subject “known to respond” are used interchangeably and refer to a subject that when administered a therapy displays an improvement in at least one criteria of the disease being treated by the therapy or does not show an increase in severity of the disease. In some embodiments, a responder is a subject that when administered a therapy displays an improvement in the disease that is being treated by the therapy. In some embodiments, a responder is a subject that when administered a therapy does not show an increase in severity of the disease. In some embodiments, an increase is severity is over time. In some embodiments, does not show an increase in severity is stable disease. In some embodiments, a responder is a subject that when administered a therapy show mixed response. In some embodiments, a responder is a subject that when administered a therapy show mixed response, wherein mixed response is improvement in at least one criteria of the disease but does not show an improvement in other criteria of the disease. In some embodiments, mixed response is shrinkage of some lesions in combination with growth of new or existing lesions. In some embodiments, a responder is a subject for which the therapy produces an anti-disease response. In some embodiments, for a subject with cancer, a responder is a subject in which the therapy produces an anticancer response. In some embodiments, a response is not a reduction in side effects. In some embodiments, a response is a reduction in side effects. In some embodiments, a response is a response against the disease itself. In some embodiments, an anticancer response is an antitumor response. In some embodiments, an antitumor response comprises tumor regression. In some embodiments, an antitumor response comprises tumor shrinkage. In some embodiments, an antitumor response comprises a lack of tumor growth. In some embodiments, an antitumor response comprises a lack of tumor metastasis. In some embodiments, an antitumor response comprises a lack of tumor hyperproliferation. In some embodiments, an improvement is in at least one symptom of the disease. In some embodiments, response is complete response. In some embodiments, response is minimal response. In some embodiments, response is partial response. In some embodiments, response comprises stable disease. In some embodiments, responder is a subject with a favorable response to the therapy. In some embodiments, non-responder is a subject with a non-favorable response to the therapy. In some embodiments, a non-favorable response is an increase in tumor burden. Increases in tumor burden can encompass any increase in tumor size or total cancer cell number such as increase in tumor size, increase in tumor spread, increase in metastasis, increase in tumor cell proliferation or any other increase.

As used herein, a “favorable response” of the cancer patient indicates “responsiveness” of the cancer patient to the treatment with the therapy, namely, the treatment of the responsive cancer patient with the therapy will lead to the desired clinical outcome such as tumor regression, tumor shrinkage or tumor necrosis; reduction in tumor burden; an anti-tumor response by the immune system; preventing or delaying tumor recurrence, tumor growth or tumor metastasis. In some embodiments, the subject is complete responder or treatment with the cancer therapy leads to stable disease. In some embodiments, a complete responder is a subject in which there is an absence of detectable cancer after treatment with the therapy. In this case, it is possible and advised to continue the treatment of the responsive cancer patient with the therapy or if the patient is cancer free to discontinue treatment. In some embodiments, the method further comprises continuing to administer the therapy to a subject that is not a non-responder. In some embodiments, the subject is non-responder, a minimal responder, partial responder or has a stable disease, and the method further comprises continuing to administer the therapy to a subject, as well as treating the subject with an additional therapy (e.g., determined using the resistance associated protein (RAP) analysis provided herein) to increase responsiveness. In some embodiments, a subject that is not a non-responder is a responder.

As used herein, the term “non-responder” and a subject “known to not respond” are used interchangeably and refer to a subject that when administered a therapy displays no improvement or stabilization in disease. In some embodiments, a non-responder displays a worsening of disease when administered a therapy. In some embodiments, non-responder is not a subject that experiences a side effect of the therapy. In some embodiments, a non-responder is a subject in which the disease progresses. In some embodiments, a non-responder is a subject in which the disease does not stabilize after therapy. In some embodiments, a non-responder is a subject in which the disease does not improve after therapy. In some embodiments, a non-responder is a subject that is not a responder as defined hereinabove. In some embodiments, a non-responder is a subject with a non-favorable response to the therapy. In some embodiments, a non-responder is a subject resistant to the therapy. In some embodiments, a non-responder is a subject refractory to the therapy.

As used herein a “non-favorable response” of the cancer patient indicates “non-responsiveness” of the cancer patient to the treatment with the therapy and thus the treatment of the non-responsive cancer patient with the therapy will not lead to the desired clinical outcome, and potentially to a non-desired outcomes such as tumor expansion, recurrence, or metastases. In some embodiments, the method further comprises discontinuing administration of the therapy to a subject that is a non-responder. In some embodiments the method further comprises continuing to administer the therapy to a subject, in combination with an additional therapy. In some embodiments, the additional therapy increases responsiveness of a non-responsive patient.

In some embodiments, the method is for determining whether the response is considered a durable response (e.g., a progression-free survival of more than 6 months).

In some embodiments, the method further comprises administering the therapy to the subject predicted to respond to the therapy. In some embodiments, the method further comprises continuing to administering the therapy to the subject predicted to respond to the therapy. In some embodiments, the method further comprises not administering the therapy to the subject predicted to not respond to the therapy. In some embodiments, the method further comprises discontinuing the therapy to the subject predicted to not respond to the therapy. In some embodiments, the method further comprises administering an alternative therapy to the subject predicted to be a non-responder. In some embodiments, the alternative therapy is an additional therapy. In some embodiments, the method further comprises administering the therapy or continuing to administer the therapy in combination with an agent or therapy that blocks or inhibits at least one of the resistance-associated factors in the subject predicted to be resistant to the therapy. In some embodiments, an agent or therapy that blocks or inhibits at least one of the resistance-associated factors is an additional therapy. In some embodiments, the combination therapy is administered to a subject predicted to be a non-responder.

In some embodiments, the method further comprises administering to the subject (e.g., a non-responder) an agent that modulates the at least one factor. In some embodiments, modulates comprises inhibits, blocks and regulates. In some embodiments, modulates is inhibits. In some embodiments, the method further comprises administering to the subject (e.g., a non-responder) an agent that modulates a pathway that comprises the at least one factor. In some embodiments, modulating the at least one factor is modulating a pathway comprising the at least one factor. In some embodiments, modulating a pathway comprising modulating a driver protein/gene that controls the at least one factor. In some embodiments, modulating a pathway comprising modulating a driver protein/gene that controls the pathway. In some embodiments, modulating a pathway comprising the at least one factor is modulating a receptor of the factor (e.g., using a receptor agonist or antagonists), a ligand or the factor, a paralog of the factor, or a combination thereof. In some embodiments, the modulating is modulating a plurality of factors. In some embodiments, the modulating is modulating a plurality of factors in the signature. In some embodiments, the modulation is modulating each factor in the signature. In some embodiments, the modulation achieves better response to therapy. In some embodiments the factor is a resistance-associated factor.

In some embodiments, a resistance score is a RAP score. In some embodiments, a resistance score is a response score. In some embodiments, a resistance score is 1-response score. In some embodiments, response score is 1-resistance score. In some embodiments, resistance score is total resistance score. In some embodiments, response score is total response score. In some embodiments, a RAP score is a total RAP score. In some embodiments, the resistance score is based on similarity of the factor expression level in the subject to the factor expression level in the non-responders. In some embodiments, the resistance score is based on similarity of the factor expression level in the subject to the factor expression level in the responders. In some embodiments, based on is calculated based on. In some embodiments, similarity is lack of similarity. In some embodiments, similarity to responders is lack of similarity to non-responders. In some embodiments, similarity to non-responders is lack of similarity to responders. In some embodiments, similarity is measured on a scale. In some embodiments, the scale is from 0 to 1, wherein 1 is perfectly similar to non-responders and 0 is perfectly similar to responders. In some embodiments, the resistance score is from 0 to 1, wherein 1 is perfectly similar to non-responders and 0 is perfectly similar to responders. In some embodiments, the resistance score is based on similarity of the factor expression level in the subject to the factor expression level in the non-responders and the factor expression level in the responders.

In some embodiments, the method comprises before step (b) selecting a subset of factor. In some embodiments, before step (b) is before the calculating. In some embodiments, the subset is a subject of the plurality of factors. In some embodiments, the subject comprises the factors that best differentiate between the responders and non-responders. In some embodiments, the factors that best differentiation are the top percentage. In some embodiments, the top percentage is the top 1, 3, 5, 10, 15, 20, 25, 30, 35, 40, 45 or 50% of factors. Each possibility represents a separate embodiment of the invention. In some embodiments, the top percentage is the top 20%. In some embodiments, the top factors are the top 10, 20, 25, 30, 40, 50, 60, 70, 75, 80, 90 or 100 factors. Each possibility represents a separate embodiment of the invention. In some embodiments, the top factors are the top 50 factors. In some embodiments, selection comprises applying a Kolmogorov-Smirnov test. In some embodiments, the Kolmogorov-Smirnov test is applied to the received factor expression levels. In some embodiments, the Kolmogorov-Smirnov test determines how well a factor differentiates between responders and non-responders. In some embodiments, the Kolmogorov-Smirnov test outputs a measure of how well a factor differentiates and the best factors are the factors with the highest scores. In some embodiments, selection comprises applying an XGBoost algorithm. In some embodiments, the calculating is for the subset. In some embodiments, the calculating is for each factor of the subset.

In some embodiments, calculating comprises applying a machine learning algorithm. In some embodiments, calculating comprises applying a machine learning model. In some embodiments, the machine learning model is a machine learning algorithm. In some embodiments, the machine learning model implements a machine learning algorithm. In some embodiments, the algorithm is a classifier. In some embodiments, the algorithm is a regression model. In some embodiments, the algorithm is supervised. In some embodiments, the algorithm is unsupervised. In some embodiments, the machine learning algorithm is trained on the expression levels in responders. In some embodiments, the machine learning algorithm is trained on the expression levels in non-responders. In some embodiments, the machine learning algorithm is trained on the expression levels in responders and non-responders. In some embodiments, the machine learning algorithm is trained on a training set. In some embodiments, the machine learning algorithm is trained by a method of the invention. In some embodiments, a machine learning algorithm is applied to factors of the plurality of factors. In some embodiments, a machine learning algorithm is applied to each factor of the plurality of factors. In some embodiments, a machine learning algorithm is applied to the subset. In some embodiments, a machine learning algorithm is applied to the subset of factors. In some embodiments, a machine learning algorithm is applied to each factor of the subset of factors. In some embodiments, each factor is analyzed and calculated separately, and the machine learning algorithm does not use expression levels of more than one factor as the training set. In some embodiments, a trained machine learning algorithm is applied to individual protein expression levels from the subject. In some embodiments, a machine learning algorithm trained on expression levels of a specific factor in responders and non-responders is applied to the expression level of that specific factor in the subject. It will be understood by a skilled artisan, that for each of the factors of the plurality of factors, a different algorithm will be trained and then applied to each expression level of the subject. Thus, if three algorithms are separately trained on expression in responders and non-responders for Factor A, Factor B and Factor C, then the algorithm trained on Factor A expression levels will be applied to the subject's expression level of Factor A, the algorithm trained on Factor B expression levels will be applied to the subject's expression level of Factor B, and the algorithm trained on Factor C expression levels will be applied to the subject's expression level of Factor C. In some embodiments, during a training phase, the machine learning model is trained on a training set comprising expression data for a single factor from responders and non-responders, using corresponding annotations of “responder” or “non-responder” to predict or classify factor expression data according to classes “responder” and “non-responder”. In some embodiments, during an inference stage, the machine learning model is applied to expression data of the single factor from a subject to predict classification of the factor as similar to a responder or non-responder. In some embodiments, the classification is a resistance score. In some embodiments, the classification is a response score. In some embodiments, the classification is a measure of how similar the factor is to non-responders and dissimilar to responders.

In some embodiments, the trained machine learning algorithm is trained to predict responsiveness of subjects suffering from the disease to the therapy. In some embodiments, the trained machine learning algorithm is trained to output a resistance score. In some embodiments, the trained machine learning algorithm is trained to output a resistance probability. In some embodiments, the trained machine learning algorithm is trained to output an activity score. In some embodiments, the trained machine learning algorithm is trained to predict activity of a resistance-associated factor in a subject. In some embodiments, the trained machine learning algorithm is trained to predict if a factor is a resistance-associated factor in the subject. In some embodiments, the trained machine learning algorithm is trained to predict if a factor of the subject is a resistance-associated factor in the subject.

In some embodiments, the training set comprises received factor expression levels. In some embodiments, the training set comprises received factor expression levels in both responders and non-responders. In some embodiments, the training set comprises received factor expression levels for only one factor. In some embodiments, the training set comprises the number of resistance-associated factors expressed in samples. In some embodiments, the sample are from subjects suffering from the disease. In some embodiments, the sample are from responders. In some embodiments, the sample are from non-responders. In some embodiments, the training set comprises at least one clinical parameter. In some embodiments, the clinical parameter is from subjects. In some embodiments, subjects are responders and non-responders. In some embodiments, the training set comprises labels. In some embodiments, the labels are associated with the responsiveness of the subjects. In some embodiments, the labels are responder or non-responder. In some embodiments, the resistance-associated factors are labeled with the labels. In some embodiments, the at least one clinical parameter is labeled with the label.

According to some embodiments, the training set further comprises at least one clinical parameter of each responder and non-responder and the machine learning algorithm is applied to individual received factor expression levels from the subject and the subject's at least one clinical parameter. In some embodiments, the at least one clinical parameter is the sex of the subjects. In some embodiments, the training set further comprises the sex of the subjects. In some embodiments, the subjects are each subject. In some embodiments, sex is gender. In some embodiments, the at least one clinical parameter is sex. In some embodiments, sex is a subject's sex. In some embodiments, sex is male or female. In some embodiments, sex is sex at birth. In some embodiments, the clinical parameter is age. In some embodiments, age is a subject's age. In some embodiments, the clinical parameter is the line of treatment. In some embodiments, the line of treatment parameter is whether the therapy was a first line of treatment or an advanced treatment. In some embodiments, a line of treatment is first line treatment. In some embodiments, a line of treatment is a secondary treatment. In some embodiments, secondary treatment is an advanced treatment. It will be understood by a skilled artisan that advanced treatment may be any line of treatment after the first, e.g., second line, third line, fourth line, fifth line, etc. In some embodiments, the clinical parameter is whether the treatment is a first line treatment or an advanced treatment. In some embodiments, the clinical parameter is PD-L1 status. In some embodiments, PD-L1 status is PD-L1 status of the cancer. Methods of measuring PD-L1 levels in cancer cells (e.g., a tumor) are well known in the art and any such method may be employed. In some embodiments, PD-L1 status comprises high PD-L1 or low PD-L1. In some embodiments, PD-L1 status comprises high PD-L1, low PD-L1 or no PD-L1. In some embodiments, PD-L1 status comprises high PD-L1, medium PD-L1 or low PD-L1. In some embodiments, PD-L1 status comprises PD-L1 expression in less than 1% of cancer cells, in 1-49% of cancer cells, or in 50% or more of cancer cells. In some embodiments, PD-L1 expression in less than 1% of cancer cells is no PD-L1 expression. In some embodiments, PD-L1 expression in less than 1% of cancer cells is low PD-L1 expression. In some embodiments, PD-L1 expression in 1-49% of cancer cells is low PD-L1 expression. In some embodiments, PD-L1 expression in 1-49% of cancer cells is medium PD-L1 expression. In some embodiments, PD-L1 expression in 50% or more of cancer cells is high PD-L1 expression.

In some embodiments, the clinical parameter is a known biomarker of the disease or mutations in known biomarkers of the disease. In some embodiments, the biomarker is selected from MYC, NOTCH, EGFR, HER2, BRAF, KRAS, MAP2K1, MET, NRAS, NTRK1, NTRK2, NTRK3, PIK3CA, RET, ROS1, TP53, ALK, CDKN2A, KIT, NF1, BFAST, FGFR, LDH, PTEN, RB1, PD-L1, MSI (Microsatelite Instability), TMB (Tumor Mutational Burden), or a combination thereof. In some embodiments, the clinical parameter is expression of the biomarker. In some embodiments, expression is percent expression. In some embodiments, expression is mutational status.

In some embodiments, the training set further comprises the sex, age and PD-L1 status of each responder and non-responder. In some embodiments, the training set further comprises the sex of each responder and non-responder. In some embodiments, the training set further comprises the age and PD-L1 status of each responder and non-responder. In some embodiments, the machine learning algorithm is applied to individual received factor expression levels from the subject and the subject's sex. In some embodiments, the machine learning algorithm is applied to individual received factor expression levels from the subject and the subject's sex, age and PD-L1 status. In some embodiments, the calculating comprises applying a machine learning algorithm trained on a training set comprising the received factor expression levels in responders and non-responders and at least one clinical parameter, to the expression levels from the subject and the subject's at least one clinical parameter and wherein the machine learning algorithm outputs the resistance score. In some embodiments, the training comprises the received factor expression levels in responders and non-responders and clinical parameters of each responder and non-responder and the machine learning algorithm is applied to individual received factor expression levels from the subject and the subject's clinical parameters and wherein the machine learning algorithm outputs response prediction. In some embodiments, the training comprises the received factor expression levels in responders and non-responders and a clinical parameter selected from sex, age and PD-L1 expression, or any combination thereof, of each responder and non-responder and the machine learning algorithm is applied to individual received factor expression levels from the subject and the subject's clinical parameters and wherein the machine learning algorithm outputs response prediction. In some embodiments, the training set comprises the number of resistance associated factors in each responder and non-responder and at least one clinical parameter and the machine learning algorithm is applied to the number of resistance associated factors from the subject and the subject's at least one clinical parameters and wherein the machine learning algorithm outputs a response prediction. In some embodiments, the training set comprises the number of resistance associated factors in each responder and non-responder and sex of each responder and non-responder and the machine learning algorithm is applied to the number of resistance associated factors from the subject and the subject's sex and wherein the machine learning algorithm outputs a response prediction. In some embodiments, the training set comprises the number of resistance associated factors in each responder and non-responder, age and PD-L1 status of each responder and non-responder and the machine learning algorithm is applied to the number of resistance associated factors from the subject and the subject's age and PD-L1 status and wherein the machine learning algorithm outputs a response prediction.

In some embodiments, the clinical parameter is the type of treatment. In some embodiments, the clinical parameter is expression of a target of the therapy. In some embodiments, the clinical parameter is expression of a protein within a process that is a target of the therapy. In some embodiments, the process is a process comprising the target of the therapy. In some embodiments, expression is expression in the subject. In some embodiments, expression is expression in a diseased tissue. In some embodiments, expression is expression in a diseased tissue sample. In some embodiments, expression is expression in the tumor. In some embodiments, expression is expression in a tumor sample. In some embodiments, a tumor sample is a biopsy. In some embodiments, expression is expression not in the tumor. In some embodiments, expression is expression not in a tumor sample. In some embodiments, expression is expression in a liquid biopsy. In some embodiments, expression is percent expression. In some embodiments, percent is percent of cells. In some embodiments, the therapy is anti-PD-1 therapy and the protein in the process is PD-L1. In some embodiments, the therapy is anti-PD-L1 therapy, and the target protein is PD-L1. In some embodiments, the clinical parameter is PD-L1 expression. In some embodiments the training set comprises at least one clinical parameter selected from line of treatment, PD-L1 expression, sex and age. In some embodiments the training set comprises protein expression levels and sex. In some embodiments the training set comprises number of RAPs, age and PD-L1 status.

Additionally clinical parameters may also be included. A skilled artisan will be able to select relevant clinical parameters for inclusion in the training set. Examples of additional clinical parameters include, but are not limited to, histological type of the sample (e.g., adenocarcinoma, squamous cell carcinoma, etc.), metastatic location, tumor location, cancer staging (such as tumor, nodes and metastases, TNM, staging for example), performance status (such as ECOG performance status), genetic mutations, epigenetic status, general medical history, vital signs, blood measurements, renal and liver function, weight, height, pulse, blood pressure and smoking history.

In some embodiments, at an inference stage the trained machine learning algorithm is applied. In some embodiments, the trained machine learning algorithm is applied to individual received factor expression levels. In some embodiments, the trained machine learning algorithm is applied to individual received factor expression levels and the at least one clinical parameter. In some embodiments, the trained machine learning algorithm is applied to individual received factor expression levels from the subjects and the subject's sex. In some embodiments, the trained machine learning algorithm is applied to the number of resistance-associated proteins. In some embodiments, the trained machine learning algorithm is applied to the number of resistance-associated factors. In some embodiments, the trained machine learning algorithm is applied to the number of resistance-associated factors and at least one clinical parameter.

In some embodiments, at the inference stage an input is received. In some embodiments, the input comprises the number of resistance-associated factors expressed in a sample. In some embodiments, the sample is from a subject. In some embodiments, the input comprises at least one clinical parameter. In some embodiments, the subject suffers from the disease. In some embodiments, the subject has unknown responsiveness to the therapy. In some embodiments, the parameter is of the subject with unknown responsiveness. In some embodiments, at the inference stage the trained machine learning algorithm is applied. In some embodiments, applied is applied to the input. In some embodiments, the input is the received input. In some embodiments, the inference stage is to predict responsiveness. In some embodiments, responsiveness is responsiveness to the therapy of the subject with unknown responsiveness.

In some embodiments, the machine learning algorithm outputs the resistance score. In some embodiments, the outputted resistance score is scaled from 0 to 1. In some embodiments, 1 is perfectly similar to non-responders and 0 is perfectly similar to responders. In some embodiments, the machine learning algorithm calculates similarity to responders. In some embodiments, the machine learning algorithm calculates similarity to non-responders. In some embodiments, the machine learning algorithm outputs a numeric value of similarity to responders and non-responders. In some embodiments, a protein is considered to be a RAP if its resistance score is beyond a certain threshold. In some embodiments, the threshold for the resistance score is calculated on a scale of 0 to 1. In some embodiments, the threshold for the resistance score of a certain protein is between 0.2 and 0.95. In some embodiments, the threshold for the resistance score of a certain protein is about 0.2, 0.25, 0.3, 0.35, 0.4, 0.42, 0.45, 0.5, 0.55, 0.6, 0.65, 0.7, 0.75, 0.8, 0.85, 0.9, or 0.95. Each possibility represents a separate embodiment of the invention. In some embodiments, the threshold for the resistance score is 0.25. In some embodiments, the threshold for the resistance score is 0.42. In some embodiments, the threshold for the resistance score is 0.6. In some embodiments, the threshold for the resistance score when calculated by a machine learning algorithm is about 0.2, 0.25, 0.3, 0.35, 0.4, 0.42, 0.45, 0.5, 0.55, 0.6, 0.65, 0.7, 0.75, 0.8, 0.85, 0.9, or 0.95. Each possibility represents a separate embodiment of the invention. In some embodiments, the threshold for the resistance score when calculated with a machine learning algorithm is 0.25. In some embodiments, the threshold for the resistance score when calculated with a machine learning algorithm is 0.42. In some embodiments, the threshold for the resistance score when calculated with a machine learning algorithm is 0.6.

In some embodiments, response probability is determined by the calculation (1-resistance score). In some embodiments, 1-resistance score is 1-final resistance score. In some embodiments, the resistance score is the final resistance score. In some embodiments, response probability is a response score. In some embodiments, the machine learning algorithm outputs the response score. In some embodiments, the outputted response score is scaled from 0 to 1. In some embodiments, 1 is perfectly similar to responders and 0 is perfectly similar to non-responders. In some embodiments, the machine learning algorithm calculates similarity to responders. In some embodiments, the machine learning algorithm calculates similarity to non-responders. In some embodiments, the machine learning algorithm outputs a numeric value of similarity to responders and non-responders. In some embodiments, a protein is considered to be a RAP if its response score is beyond a certain threshold. In some embodiments, the threshold for the response score is calculated on a scale of 0 to 1. In some embodiments, the threshold for the response score of a certain protein is between 0.2 and 0.95. In some embodiments, the threshold for the response score of a certain protein is about 0.2, 0.25, 0.3, 0.35, 0.4, 0.42, 0.45, 0.5, 0.55, 0.6, 0.65, 0.7, 0.75, 0.8, 0.85, 0.9, or 0.95. Each possibility represents a separate embodiment of the invention. In some embodiments, the threshold for the response score is 0.25. In some embodiments, the threshold for the response score is 0.42. In some embodiments, the threshold for the response score is 0.6. In some embodiments, the threshold for the response score when calculated by a machine learning algorithm is about 0.2, 0.25, 0.3, 0.35, 0.4, 0.42, 0.45, 0.5, 0.55, 0.6, 0.65, 0.7, 0.75, 0.8, 0.85, 0.9, or 0.95. Each possibility represents a separate embodiment of the invention. In some embodiments, the threshold for the response score when calculated with a machine learning algorithm is 0.25. In some embodiments, the threshold for the response score when calculated with a machine learning algorithm is 0.42. In some embodiments, the threshold for the response score when calculated with a machine learning algorithm is 0.6. In some embodiments, the algorithm outputs response probability, and the response probability is calculated on a scale of 0 to 1. In some embodiments, the algorithm outputs response probability, and the response probability is calculated on a scale of 0% to 100%, wherein 100% is responder and 0% is non-responder.

In some embodiments, the score is between zero and 1. In some embodiments, active is active in the cancer. In some embodiments, active is active in the subject. In some embodiments, active is active in promoting resistance. In some embodiments, beyond a threshold is below a threshold. In some embodiments, beyond a threshold is above a threshold. In some embodiments, the predetermined threshold is 0.5, 0.4, 0.3, 0.25, 0.2, 0.15, 0.1, 0.05, 0.01, 0.005, 0.001, 0.0005 or 0.0001. Each possibility represents a separate embodiment of the invention. In some embodiments, the threshold is 0.05. In some embodiments, the threshold is 5%.

In some embodiments, the machine learning algorithm outputs the resistance score. In some embodiments, the resistance score is the RAP score. In some embodiments, the outputted resistance score is scaled from 0 to 1. In some embodiments, 1 is perfectly similar to non-responders and 0 is perfectly similar to responders. In some embodiments, the machine learning algorithm calculates similarity to responders. In some embodiments, the machine learning algorithm calculates similarity to non-responders. In some embodiments, the machine learning algorithm outputs a numeric value of similarity to responders and non-responders. In some embodiments, a protein is considered to be a RAP if its resistance score is beyond a certain threshold. In some embodiments, the threshold for the resistance score is calculated on a scale of 0 to 1. In some embodiments, the threshold for the resistance score of a certain protein is between 0.2 and 0.95. In some embodiments, the threshold for the resistance score of a certain protein is about 0.01, 0.05, 0.1, 0.15, 0.2, 0.25, 0.3, 0.35, 0.4, 0.42, 0.45, 0.5, 0.55, 0.6, 0.65, 0.7, 0.75, 0.8, 0.85, 0.9, or 0.95. Each possibility represents a separate embodiment of the invention. In some embodiments, the threshold for the resistance score is 0.25. In some embodiments, the threshold for the resistance score is 0.42. In some embodiments, the threshold for the resistance score is 0.6. In some embodiments, the threshold for the resistance score when calculated by a machine learning algorithm is about 0.2, 0.25, 0.3, 0.35, 0.4, 0.42, 0.45, 0.5, 0.55, 0.6, 0.65, 0.7, 0.75, 0.8, 0.85, 0.9, or 0.95. Each possibility represents a separate embodiment of the invention. In some embodiments, the threshold for the resistance score when calculated with a machine learning algorithm is 0.25. In some embodiments, the threshold for the resistance score when calculated with a machine learning algorithm is 0.42. In some embodiments, the threshold for the resistance score when calculated with a machine learning algorithm is 0.6.

In some embodiments, response probability is determined by the calculation (1-resistance score). In some embodiments, 1-resistance score is 1-final resistance score. In some embodiments, the resistance score is the final resistance score. In some embodiments, response probability is a response score. In some embodiments, the machine learning algorithm outputs the response score. In some embodiments, the outputted response score is scaled from 0 to 1. In some embodiments, 1 is perfectly similar to responders and 0 is perfectly similar to non-responders. In some embodiments, the machine learning algorithm calculates similarity to responders. In some embodiments, the machine learning algorithm calculates similarity to non-responders. In some embodiments, the machine learning algorithm outputs a numeric value of similarity to responders and non-responders. In some embodiments, a protein is considered to be a RAP if its response score is beyond a certain threshold. In some embodiments, beyond is above. In some embodiments, beyond is below. In some embodiments, the threshold for the response score is calculated on a scale of 0 to 1. In some embodiments, the threshold for the response score of a certain protein is between 0.2 and 0.95. In some embodiments, the threshold for the response score of a certain protein is about 0.2, 0.25, 0.3, 0.35, 0.4, 0.42, 0.45, 0.5, 0.55, 0.6, 0.65, 0.7, 0.75, 0.8, 0.85, 0.9, or 0.95. Each possibility represents a separate embodiment of the invention. In some embodiments, the threshold for the response score is 0.25. In some embodiments, the threshold for the response score is 0.42. In some embodiments, the threshold for the response score is 0.6. In some embodiments, the threshold for the response score when calculated by a machine learning algorithm is about 0.2, 0.25, 0.3, 0.35, 0.4, 0.42, 0.45, 0.5, 0.55, 0.6, 0.65, 0.7, 0.75, 0.8, 0.85, 0.9, or 0.95. Each possibility represents a separate embodiment of the invention. In some embodiments, the threshold for the response score when calculated with a machine learning algorithm is 0.25. In some embodiments, the threshold for the response score when calculated with a machine learning algorithm is 0.42. In some embodiments, the threshold for the response score when calculated with a machine learning algorithm is 0.6.

In some embodiments, the machine learning model is a machine learning algorithm. In some embodiments, the algorithm is a supervised learning algorithm. In some embodiments, the algorithm is an unsupervised learning algorithm. In some embodiments, the algorithm is a reinforcement learning algorithm. In some embodiments, the machine learning model is a Convolutional Neural Network (CNN). In some embodiments, the at least one hardware processor trains a machine learning model. In some embodiments, the model is based, at least in part, on a training set. In some embodiments, the model is based on a training set. In some embodiments, the model is trained on a training set. In some embodiments, the at least one hardware processor applies the machine learning model to a factor expression level from a subject.

In some embodiments, the calculating comprises calculating a mean expression for each protein in responders. In some embodiments, the calculating comprises calculating a mean expression for each protein in non-responders. In some embodiments, the calculating comprises calculating a mean expression for each protein in responders and a mean expression for each protein in non-responders. In some embodiments, the calculating comprises calculating a distribution of the expression for each protein in responders and non-responders. In some embodiments, the calculating comprises calculating a standard deviation of expression for each protein in responders and non-responders. In some embodiments, in responders is in the responders population. In some embodiments, in non-responders is in the non-responders population. In some embodiments, the resistance score is based on the ratio of deviation of the factor expression in the subject from the calculated mean in responders to the deviation of the factor expression in the subject from the calculated mean in non-responders. Calculation of deviation is well known to one skilled in the art. It will be understood that the more dissimilar the expression in the subject is from a mean the larger the deviation will be. Thus, factors that are very dissimilar to the mean in responders will have a large numerator in the calculation of this ratio and factors that are lowly dissimilar to the mean in non-responders will have a small denominator. Thus, the more dissimilar to responder expression and the more similar to non-responder expression is expression of a factor in a subject the higher the resistance score will be. In some embodiments, a resistance score beyond a predetermined threshold indicates a factor is a resistance-associated factor. In some embodiments, a resistance-associated factor is a resistance-associated protein (RAP).

In some embodiments, the calculating further comprises calculating a distribution for each factor in responders. In some embodiments, the calculating further comprises calculating a distribution for each factor in non-responders. In some embodiments, the calculating further comprises calculating a distribution for each factor in responders and a distribution for each factor in non-responders. In some embodiments, the calculating further comprises calculating a standard deviation for each factor in responders. In some embodiments, the calculating further comprises calculating a standard deviation for each factor in non-responders. In some embodiments, the calculating further comprises calculating a standard deviation for each factor in responders and a standard deviation for each protein in non-responders. In some embodiments, the calculating further comprises calculating a standard deviation for each factor in a mix of responders and non-responders. In some embodiments, the deviation is measured as a multiple of the calculated standard deviation. It will be understood by a skilled artisan that by scaling the deviation to the standard deviation for a group of expression values the deviation can be given in more absolute terms allow for the comparison of factors and populations with very small and very large stand deviations (which may also have very low and very high expression levels).

In some embodiments, the resistance score is based on a Z-score for the expression level of each factor in the subject. In some embodiments, the resistance score is based on the Z-score relative to responders. In some embodiments, the resistance score is based on the Z-score relative to non-responders. In some embodiments, the resistance score is based on both the Z-score relative to responders and the Z-score relative to non-responders. In some embodiments, the resistance score is based on the ratio of the Z-score relative to responders to the Z-score relative to non-responders. It will be well known to a skilled artisan that a Z-score counts the distance of the individual level from the population mean in units of the population standard deviation. In some embodiments, the Z-score is calculated by Equation 1.

In some embodiments, the resistance score is calculated by the equation (|Z_{R}|/|Z_{NR}|+c). In some embodiments, Z_{R} is the deviation of the factor expression in the subject from the calculated mean in responders. In some embodiments, Z_{NR} is the deviation of the factor expression in the subject from the calculated mean in non-responders. In some embodiments, ∥ is the Z-score of the deviation. In some embodiments, ∥ is the standardizing of the deviation to a multiple of the standard deviation. In some embodiments, c is a constant. In some embodiments, constant is a regulation constant that prevents the score from divergence for Z_{NR}=0. In some embodiments, the resistance score is calculated by Equation 2. In some embodiments, monotonoic is an ad-hoc function that prevents the resistance score from decreasing for extreme values within the non-responder distributions. In some embodiments, function is the function provided in Algorithm 1.

In some embodiments, a resistance score beyond a predetermined threshold indicates a factor is a RAP. In some embodiments, beyond is above. In some embodiments, the threshold is a predetermined threshold. In some embodiments, threshold is a threshold value. In some embodiments, the threshold for the resistance score is about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 5.0, 5.1, 5.2, 5.3, 5.4, 5.5, 5.6, 5.7, 5.8, 5.9, 6.0. 6.1, 6.2, 6.3, 6.4, 6.5, 6.6, 6.7, 6.8, 6.9, or 7.0. Each possibility represents a separate embodiment of the invention. In some embodiments, the threshold is about 0.05, 0.1, 0.15, 0.2, 0.25, 0.3, 0.35, 0.4, 0.45, 0.5, 0.55, 0.6, 0.67, 0.7, 0.75, 0.8, 0.85 or 0.9. Each possibility represents a separate embodiment of the invention. In some embodiments, the threshold for the resistance score is about 2.9. In some embodiments, the threshold for the resistance score is 2.9. In some embodiments, the threshold for the resistance score is about 3.0. In some embodiments, the threshold for the resistance score is 3.0. In some embodiments, the threshold for the resistance score is calculated on a scale of arbitrary units. In some embodiments, the threshold for the resistance score when calculated by a mathematical calculation is about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, or 5.0. Each possibility represents a separate embodiment of the invention. In some embodiments, the threshold for the resistance score when calculated with a mathematical calculation is about 2.9. In some embodiments, the threshold for the resistance score when calculated with a mathematical calculation is 2.9. In some embodiments, the threshold for the resistance score when calculated with a mathematical calculation is about 3.0. In some embodiments, the threshold for the resistance score when calculated with a mathematical calculation is 3.0. In some embodiments, a mathematical calculation is a method that comprises calculating a mean expression for each protein.

In some embodiments, a subject with a number of resistance-associated factors (e.g., RAPs) above a predetermined number is predicted to be resistant to the therapy. In some embodiments, a subject with a number of resistance-associated factors above a predetermined number is predicted to not respond to the therapy. In some embodiments, a subject with a number of resistance-associated factors above a predetermined number is predicted to be a non-responder to the therapy. In some embodiments, a subject with a number of resistance-associated factors below a predetermined number is predicted to be suitable to the therapy. In some embodiments, a subject with a number of resistance-associated factors below a predetermined number is predicted to respond to the therapy. In some embodiments, a subject with a number of resistance-associated factors below a predetermined number is predicted to be a responder to the therapy. In some embodiments, a subject with a number of resistance-associated factors at or below a predetermined number is predicted to be suitable to the therapy. In some embodiments, a subject with a number of resistance-associated factors at or below a predetermined number is predicted to respond to the therapy. In some embodiments, a subject with a number of resistance-associated factors at or below a predetermined number is predicted to be a responder to the therapy.

In some embodiments, the predetermined number is a threshold number. In some embodiments, the predetermined number is 2, 3, 4, 5, 6, 7, 8, 9, 10, 11, 12, 13, 14, 15, 16, 17, 18, 19 or 20. Each possibility represents a separate embodiment of the invention. In some embodiments, the predetermined number is 3. In some embodiments, the predetermined number is 4. In some embodiments, the predetermined number is 7. In some embodiments, the predetermined number is 13.

In some embodiments, the method further comprises classifications of the resistance-associated factors into at least one pathway, process, or network. In some embodiments, the method further comprises performing analysis on resistance associated factors to determine at least one pathway, process, or network in which the resistance-associated factors are involved. In some embodiments, the pathway, process, or network causes non-responsiveness to the therapy. In some embodiments, the analysis is selected from pathway analysis, process analysis and network analysis. In some embodiments, the method further comprises performing pathway analysis on RAPs. In some embodiments, the method further comprises performing process analysis on RAPs. In some embodiments, the method further comprises performing network analysis on RAPs. In some embodiments, at least one pathway, process or network comprises at least 2, 3, 4, 5, 6, 7, 8, 9 or 10 pathways, processes, or networks. Each possibility represents a separate embodiment of the invention. In some embodiments, at least one pathway, process or network is all the pathways, processes or networks known to include the resistance associated factors. In some embodiments, at least one pathway, process or network is all the pathways, processes or networks enriched with resistance associated factors. In some embodiments, enriched is the most enriched. In some embodiments, enriched comprises contains the most RAPs of any or the pathways, processes or networks.

In some embodiments, the method comprises selecting a pathway, process or network. In some embodiments, the selected pathway, process or network is hypothesized to affect non-response to the therapy. In some embodiments, the selected pathway, process or network is hypothesized to cause non-response to the therapy. In some embodiments, the selected pathway, process or network is known to be druggable. In some embodiments, known to be druggable comprises a known therapeutic agent that modulates the pathway, process or network. In some embodiments, the known therapeutic agent is in or has concluded clinical trials. In some embodiments, the known therapeutic agent is approved for human use. In some embodiments, approved for human use is approved for use in treating the disease in a human. In some embodiments, the disease is cancer. In some embodiments, the method further comprises administering to a subject that is a non-responder, or predicted to be a non-responder, an agent that modulates the at least one pathway, process, or network containing a resistance associated factor. In some embodiments, the agent inhibits a target in said pathway, process, or network. In some embodiments, the target is a gene. In some embodiments, the target is a protein. In some embodiments, the protein is a regulatory RNA. In some embodiments, the target is a response associated factor. In some embodiments, the target is not a response associated factor. In some embodiments, the agent activates a target in the pathway, process, or network. In some embodiments, the agent modulates the pathway, process or network. In some embodiments, the pathway's activity induces non-response, and the agent inhibits the pathway. In some embodiments, the pathway's activity reduces non-response, and the agent activates the pathway. It will be understood by a skilled artisan that a response associated factor is identified by its expression in a subject being more similar to the expression in non-responders than responders. Thus, for example, if the factor is more highly expressed in non-responders and increases activity of the pathway/process/network then the agent would inhibit the pathway. If, for example, the factor is more highly expressed in non-responders, but decreases activity of the pathway/process/network then the agent would activate the pathway/process/network. Similarly, if the factor, for example, is more lowly expressed in non-responders and decreases activity of the pathway/process/network the agent would inhibit the pathway/process/network. And lastly, if, for example, the factor is more lowly expressed in non-responders but increases activity of the pathway/process/network the agent would activate the pathway/process/network. Essentially, the agent should induce the pathway/process/network to function more as it does in responders. In some embodiments, the agent targets a hub target in the pathway. In some embodiments, the agent targets a regulator target in the pathway. In some embodiments, the process activity induces non-response, and the agent inhibits the process. In some embodiments, the processes' activity reduces non-response, and the agent activates the process. In some embodiments, the agent targets a hub target in the process. In some embodiments, the agent targets a regulator target in the process. In some embodiments, the network activity induces non-response, and the agent inhibits the network. In some embodiments, the network activity reduces non-response, and the agent activates the network. In some embodiments, the agent targets a hub factor in the network. In some embodiments, the agent targets a regulator factor in the network. In some embodiments, the regulator is a master regulator. The factors can be classified into pathways, protein interaction or signals using any analysis tool known in the art. Examples include, but are not limited to, GO analysis, Ingenuity analysis, Metacore analysis (Clarivate Analytics), reactome pathway analysis and functional analysis.

By another aspect there is provided, a computer program product comprising a non-transitory computer-readable storage medium having program code embodied thereon, the program code executable by at least one hardware processor to perform a method of the invention.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Example 1: Response Prediction Based on Resistance Associated Proteins (RAPs)—Proof of Concept

### Data Collection

The response prediction proof of concept is based on analysis of blood samples from 108 Non-Small Cell Lung Cancer (NSCLC) patients under Immune Check Inhibitor (ICI) treatment. The various treatments administered are summarized in Table 1.

TABLE 1

| Treatment | Patient count |
|---|---|
| Pembrolizumab, Chemo | 41 |
| Pembrolizumab | 37 |
| Nivolumab | 12 |
| Ipilimumab, Nivolumab | 6 |
| Treatment unknown | 3 |
| Ipilimumab, Nivolumab, Chemo | 4 |
| Atezolizumab | 3 |
| Nivolumab, Chemo | 2 |

Plasma protein levels in the 108 patients were measured, in which approximately 1100 non-redundant protein targets are measured. Samples were taken before initiation of ICI treatment (T0) and after the first treatment was administered (T1) for a total of 156 samples in the batch.

### Classifier Construction

To predict response to treatment, the proteomic levels and the response labels were incorporated by a supervised learning algorithm. The response labels were responders (R) and non-responders (NR) and were determined based on the Overall Response Rate (ORR) assessment at 3 months. Specifically, progressive disease (PD) or early death associated with disease progression was classified as NR, and Stable Disease (SD), Minimal Response (MR), Partial Response (PR) and Complete Response (CR) were classified as R. The ORR assessment was performed as described in clinical trial NCT04056247 (clinicaltrials.gov/ct2/show/NCT04056247, herein incorporated by reference in its entirety) in the “Primary Outcome Measures” section, by RECIST 1.1 or other validated method for ORR evaluation. Changes in the blood levels of different proteins that represent the host response [Time Frame: At baseline (pre-therapy) and after 1^{st} dose administration (post therapy)] were determined as described.

The samples were divided into a training set and a test set. All the development stages of the algorithm were performed using the training set while the test set was used only at the final stage to test the performance of the final algorithm. The training set included samples from n=78 patients (59 responders and 19 non-responders), and the test set included the samples analyzed in n=30 patients.

The response classifier treats features as an input and predicts response based on feature values. The features are the protein levels measured in the plasma at the two time points—at baseline (T0) and following the first treatment (T1). Measurements of the same protein at different time points are regarded as independent features. Moreover, some proteins have more than one measurement in a single proteomic profile (for example, the protein IL-6 is measured four times). Each repeat was treated as an independent feature.

### Resistance Associated Proteins

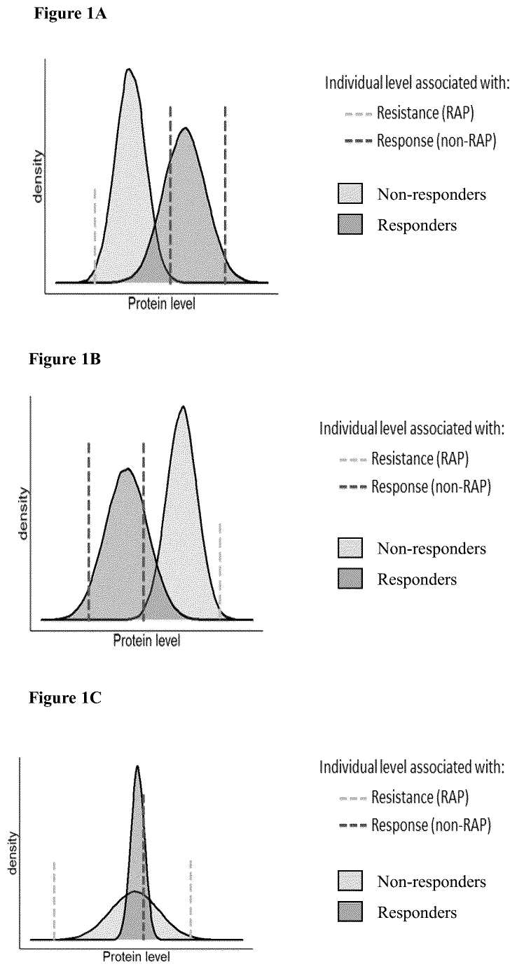

A resistance associated protein (RAP) refers to a specific protein whose expression in a given patient confers resistance to therapy, i.e., RAPs are patient specific. A protein is considered to be a RAP when its expression level in the respective patient is more similar to its expression distribution in the non-responder population than to the responder population (see FIGS. 1A-1C for illustrations). RAPs can be determined in a variety of ways. Provided herein is a mathematical calculation of RAPs as well as a machine learning algorithm for classifying RAPs and a method that combines the two. These methods are merely exemplary and any method of calculating RAPs may be employed.

To put the above concept into quantitative terms, a RAP score (i.e., a resistance score) was determined for each protein. A low RAP score value represents an expression level which is typical to the responder population, and a high RAP score indicates an expression level which is typical to the non-responder population. A protein is considered a RAP in cases where its RAP score is beyond (e.g., above or below depending on the construction of the score) a certain threshold. The RAP score threshold optimization process is described hereinbelow.

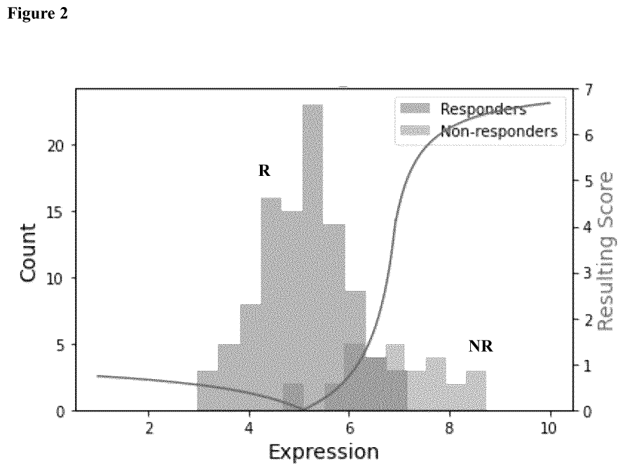

The RAP score calculation requires knowing the expression level distribution of each protein in both responder and non-responder populations, and data on the protein level expression of the tested patient. To allow comparison between several different proteins at different ranges of expression level, it is important that the RAP score will not be affected by and sensitive to the protein level expression scale. This is especially important in plasma samples, where there is a large dynamic range of 11 orders of magnitude in protein expression levels. To achieve this, the RAP score is based on Z-score, which counts the distance of the individual level from the population mean in units of the population standard deviation. In technical terms, Z-score is defined by Equation 1.

$$Z = \frac{x - μ}{σ} \qquad Equation 1$$

where x is the protein level in the tested patient, μ is the mean protein level in the population, and σ is the population standard deviation. The Z-score of a given patient is calculated separately with respect to the responders and non-responders populations. For the calculation of the Z-score relative to the responder population, noted by Z_{R}, the distribution measures, μ and σ, are calculated by using the responder population. For the calculation of the Z-score relative to the non-responder population, noted by Z_{NR}, the distribution measures, μ and σ, are calculated by using the non-responder population. Finally, the RAP score is defined by 2,

$$monotonic ( \frac{\vert {Z}_{R} \vert}{\vert {z}_{N R} \vert + c} ) \qquad Equation 2$$

where c is a regularization constant that prevents the score from divergence for Z_{NR}=0, and monotonoic is an ad-hoc function that was designed to prevent the RAP-score from decreasing for extreme values within the non-responder distributions. The function implementation is given by pseudo-code in Algorithm 1. RAP score values for representative responder and non-responder distributions are shown in FIG. 2.

Algorithm 1: The monotonic function used in Equation 2.

```
if |mean(R) − mean(NR)| > c · std(NR) then
  if mean(NR) > mean(R) then
    $sign ( mean ( NR ) - x ) \cdot RAP Score + \frac{( x > mean ( NR ) \cdot 2 \cdot \vert {Zscore}_{R} \vert )}{c}$
  else
    $sign ( x - mean ( NR ) ) \cdot RAP Score + \frac{( x < mean ( NR ) \cdot 2 \cdot \vert {Zscore}_{R} \vert )}{c} .$
```

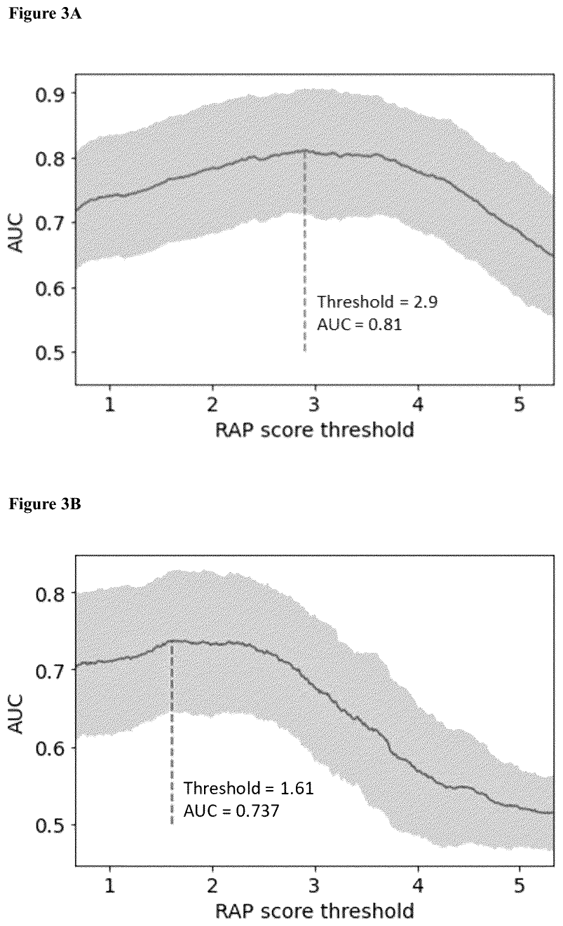

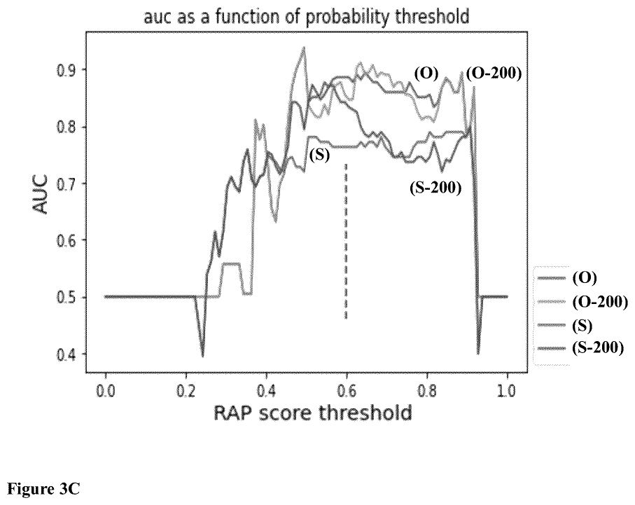

To determine the exact number of RAPs for a given patient, a threshold was determined for all proteins, wherein a protein with a RAP score above the determined threshold was considered as a RAP. The threshold was determined using cross-validation which is applied on the training set. Specifically, a cross-validation data set consisting of one third of the training set and a non-cross validation data set consisting of an additional one-third of the training set were sampled, while keeping the number of responders and non-responders similar between cross-validation and non-cross validation data sets. The calculation was performed on the non-cross-validation set and then for each patient in the cross-validation data set, a RAP score was calculated for every feature (i.e., all measured proteins at T0 and T1) using the responder and non-responder expression level distributions. The number of RAPs was then used to predict the response and receiver operating characteristics (ROC) area under the curve (AUC) quantifying the prediction performance was calculated for each threshold value (FIG. 3A-3B). To minimize the noise associated with a small dataset, 100 realizations were performed for each threshold value (i.e., different sampling of the cross-validation set from the training set) and the average AUC across the 100 realizations was considered. Notably, the mean ROC AUC curve of FIG. 3A demonstrates a single wide peak, suggesting that the prediction power of the number of RAPs is not very sensitive to the selected threshold. For features that include measurements at T0 and T1, the threshold was set to 1.61 (FIG. 3B) and 2.9 (FIG. 3A), respectively.

Machine learning evaluation: Although a purely mathematical approach is powerful (both conceptually and practically), it has several disadvantages that should be addressed:
1. The RAP score function depends on the underlying distribution of the protein expression level, hence its effectiveness may be platform dependent (in particular, as different proteomic systems use different measurement units that do not scale naturally).
2. The current implementation does not provide a natural way to include clinical parameters (such as patient condition, indication details, treatment details, etc.) in the predictor.

An alternative approach making use of decision tree learning based on a machine learning algorithm to classify proteins as RAPs for a given subject was invented. For each measured protein a prediction model was generated using a machine learning algorithm (e.g., XGBoost algorithm) and based on the data of the training set. Such data from the training set may include not only protein expression levels and responder/non-responder tags, but also other features such as patient age, sex, condition, type of treatment, line of treatment, biomarkers expression such as PD-L1 expression etc. This approach makes no assumptions on the protein distribution and offers a natural framework to utilize clinical parameters.

To test this approach, samples from a cohort of 76 patients were screened using two different protein analysis platforms: approximately 1200 proteins (O) and the other measuring approximately 7500 proteins(S), with about 1000 proteins being common to both platforms. The treatment administered to these subjects is summarized in Table 2.

TABLE 2

| Treatment | Patient count |
|---|---|
| Pembrolizumab | 28 |
| Pembrolizumab, Chemo | 27 |
| Nivolumab | 5 |
| NA | 3 |
| Ipilimumab, Nivolumab | 4 |
| Ipilimumab, Nivolumab, Chemo | 4 |
| Atezolizumab | 3 |
| Nivolumab, Chemo | 2 |

The cohort of the 76 patients was divided into a training set that included 51 subjects (38 responders and 13 non-responders) and a test set that included 25 subjects (19 responders and 6 non-responders). The XGBoost algorithm was selected for this analysis due to the non-linear nature of the problem and the algorithms reputation of efficiency with learning on small data sets. In order to avoid multiple comparisons on the test set that will increase the risk of false discovery, and as the study goal is to verify the prediction feasibility (rather than identifying the optimal model configuration), the following predetermined configuration was used for the training model:

Model hyperparameters were set to:
a. Max tree depth=4
b. Ridging factors: eta=0.8, lambda=5, alpha=2
c. num_parallel_tree=100
d. objective=binary: logistic
e. eval_metric=logloss
   The parameters were selected in order to handle a small, noisy data set.

For the purposes of this evaluation the machine learning algorithm was trained only on protein expression levels while other considerations were excluded. Patient expression results were evaluated for each protein separately, and protein classifier was calculated for each single protein. The machine learning algorithm outputs a score from 0 to 1, with 1 being most similar to non-responders and 0 being most similar to responders.

Two configurations of input proteins were used to evaluate this approach. In the first configuration, all proteins were used as potential predictors. This is similar to what was employed in the mathematical approach, however, while for large cohorts this method is expected to be effective, for a small cohort size (compared to the number of features) false detection may hinder the predictive capability. In the second configuration, ranking the single protein models according to their tendency to partition the patients to responders and non-responders (i.e., give a higher rank to a protein model that has more balanced prediction classes) was used. As an extreme example, if a model predicted that all patients belong to a single class (responders or non-responders) the model received the lowest possible balance rank. On the other side of the scale, a model that divided the population evenly between responders and non-responders received the highest balance rank. After ranking the different protein models, the machine learning approach was evaluated using the 200 proteins with the highest balance rank.

Both approaches were used to evaluate the subjects based on their “O” and “S” expression values at T0 and T1. The model performance for “O” (measured by AUC) was above 0.8 in the threshold range of 0.4-0.8, with a stable and smooth behavior (FIG. 3C), peaking at an AUC=0.89 and 95% confidence interval of [0.594, 0.995]. Thus, for these samples the threshold was set at about 0.6. This result is a minor improvement to the AUC=0.846 obtained for the same data set using the mathematical RAP approach. However, due to the large confidence intervals (that are a result of the small data set size) the statistical significance of this difference is moderate.

The peak model performance for “O” when restricting the predictor to the 200 proteins was AUC=0.91 and 95% confidence interval of [0.602, 0.996] (FIG. 3C). The threshold was essentially the same in this case and the AUC represents a slight improvement to the full protein set configuration.

The model performance for “S” (measured by AUC) was above 0.75 in the threshold range of 0.4-0.9, with a stable and smooth behavior (FIG. 3C), peaking at an AUC of 0.81 and 95% confidence interval of [0.587, 0.924]. Thus, for these samples the threshold could be set slightly lower, at about 0.59, although this difference may be negligible. This result is inferior to the behavior observed from the same model configuration using “O” data (about 1 standard deviation lower), which is not unexpected due to the considerably larger number of proteins and the small data set size.

The peak model performance for “S” when restricting the predictor to the 200 proteins was an AUC=0.87 and 95% confidence interval of [0.597, 0.992] (FIG. 3C). The threshold was thus essentially the same as found for the “S” analysis, and represents a considerable improvement compared to the full protein set configuration, consistent with the lowering of the false detection rate imposed by this configuration. Still, the performance of the 200 proteins configuration for “S” is slightly lower than the same configuration using “O”; however, the statistical significance of the difference (<0.3 standard deviations) is low.

### Response Prediction by RAP Number

The RAP score described above enables identifying patient-specific proteins with expression levels that correspond with non-responsiveness, as reflected by responder and non-responder expression. It was therefore hypothesized that the number of RAPs possessed by a certain patient will predict the patient's response; a patient with a small number of RAPs or no RAPs at all is expected to respond to the treatment, since almost all the measured proteins demonstrate expression levels that fit the responder population. A patient with a larger number of RAPs is expected to develop resistance since the expression level of several proteins is similar to the non-responder population. This method does not take into consideration the nature of the RAPs, and each subject may have completely different RAPs. Rather it is the total number of RAPs and not the identity of the RAPs that is important.

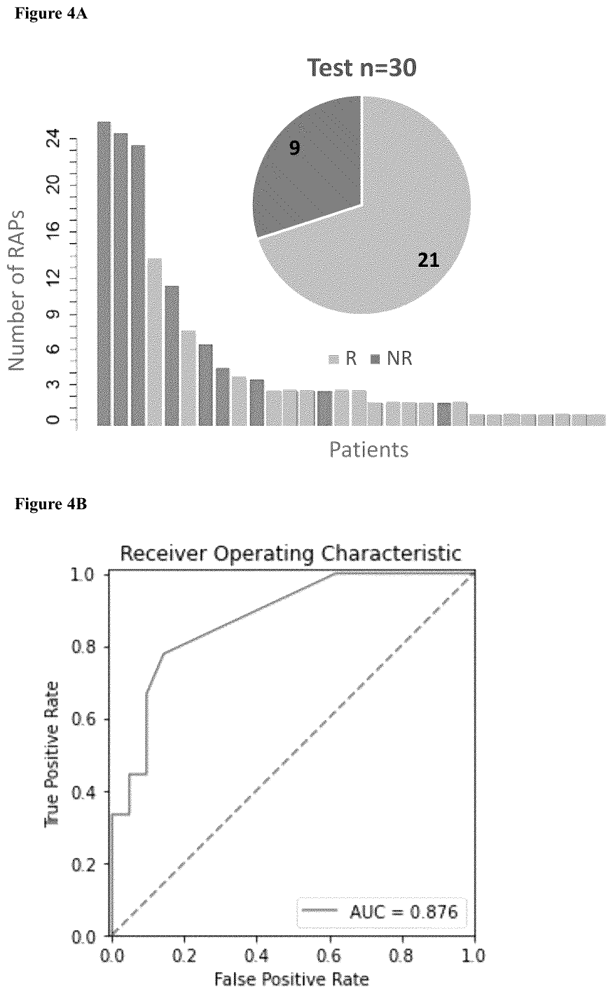

The RAP score predictive performance was tested using the test set. Specifically, for each patient in the test set (n=30), RAP score was calculated for all features using R and NR protein level distributions of all the patients in the training set (n=78). Together with the threshold, that was calculated using the training set as explained above, it is possible to infer the number and identity of each patient's RAPs in the test set. FIG. 4A shows the 30 subjects from the test set and the number of RAPs calculated (using the mathematical method) for each subject using the T0+T1 data. The threshold was set at 3 RAPs, and subjects with more than 3 RAPs were predicted to be non-responders. The ROC curve shows an AUC of 0.88 indicating that the analysis is highly predictive (FIG. 4B).

### Targeting RAPs

Improved understanding of molecular and immunologic mechanisms of resistance to ICI therapy may not only identify novel predictive biomarkers but may also suggest targets for combined ICI therapy. Combined therapies aim to selectively block ICI resistance proteins to improve ICI outcomes in non-responding patients.

In order to find targets for combined therapy, all RAPs with a score >2.9 (the defined threshold) found in the test set patients were evaluated. Next, a search for clinical trials in which RAPs from this list are targeted in combination with ICI in non-small cell lung cancer (NSCLC) patients or patients with solid tumors were examined. Mapping of clinical trials with combined therapy yielded 1300 clinical trials targeting 430 proteins in combination with ICI in NSCLC or solid tumors or by 500 different drugs. Comparing the 30 RAPs that passed the score threshold in the test set (RAPs appearing in at least one patient among the thirty patients and having score higher than 2.9) and the list of proteins found to be targeted in clinical trials in combination with ICI, revealed four RAPs that were also targeted in combination with ICI in NSCLC trials: KDR (VEGFR2), IL6, EPHA2 and TACSD2.

IL-6 is one of the targetable RAPs identified in the test set cohort of patients. Recently the inventors showed that therapeutic efficacy of anti-CTLA-4 is significantly improved by the coadministration of anti-IL-6 in tumor-bearing mice (Khononov, et al., 2021, “Host response to immune checkpoint inhibitors contributes to tumor aggressiveness”, J. Immunother. Cancer, Mar; 9) 3_: e001996; herein incorporated by reference in its entirety). These results are in line with a previous publication demonstrating improved therapeutic outcome when anti-IL-6 is combined with anti-PD1 or anti-PD-L1 treatment. Moreover, the in vitro experiments in Khononov et al., demonstrate that inhibiting IL-6 diminishes anti-PD-1-induced tumor cell invasive properties, further supporting the notion that blocking specific therapy-induced host factors represents a strategy for overcoming therapy resistance.

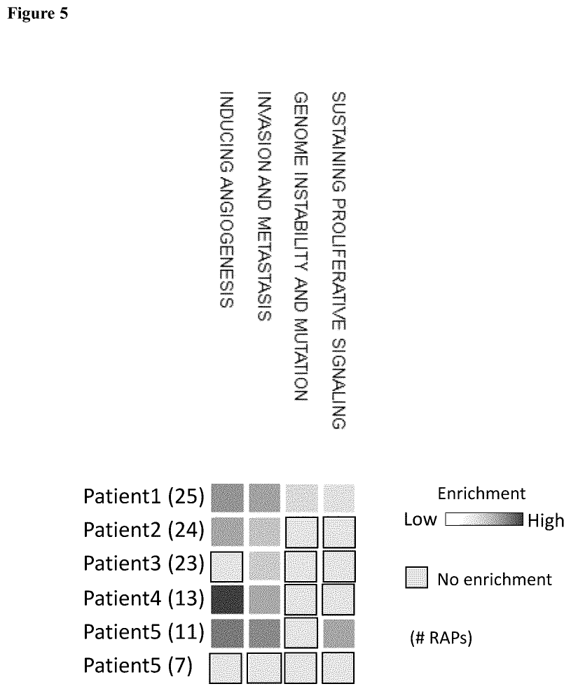

An alternative approach for therapeutic targeting based on the RAPs is by associating the proteins to main biological processes that are cancer related. To this end, each protein was assigned to hallmark/s of cancer, which capture major tumorigenic processes. Then, enrichment analysis was performed for each patient using the RAPs as an input (Fisher exact test; FIG. 5). A preliminary analysis on six patients revealed four enriched processes in total. One patient had significant enrichment in all four processes; four patients displayed enrichment of 1-3 processes; one patient did not have any significant processes.

Once the enrichment analysis is done for a patient, the treating physician can choose a therapy based on the enriched biological processes. For example, if angiogenesis is significantly enriched, the physician may choose to combine an approved drug targeting angiogenesis (e.g., Avastin) with the ICI. Another example is a patient with high proliferation signal; in this case, the physician may choose to combine ICI with a chemotherapy against tumor cell proliferation.

In order to further examine the biological aspects of the RAPs, the 19 RAPs that were obtained in at least 3 patients of the test set cohort were examined. Most patients had 4-5 RAPs. The most common RAP among the examined patients was VEGFR2 (KDR; was identified as a RAP in 12 patients). Notably, most of the RAPs were identified in T1, suggesting that resistance to therapy is mainly acquired and results from host response. VEGFR2 was identified as a RAP at both T0 and T1, though at T1 it was defined as a RAP in more patients (12 patients compared to 8 at T0). VEGFR2 is one of the two receptors of vascular endothelial growth factor (VEGF), a major growth factor for endothelial cells whose expression is higher in responders.

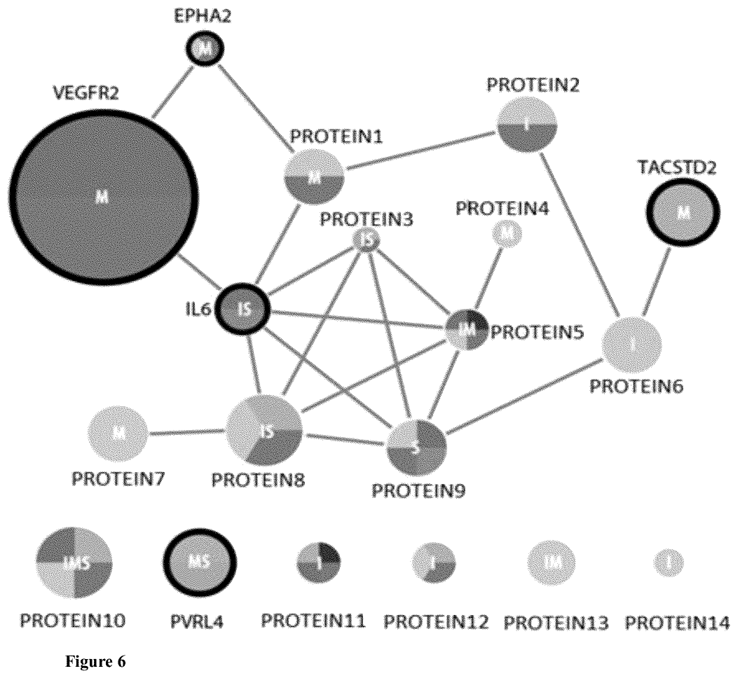

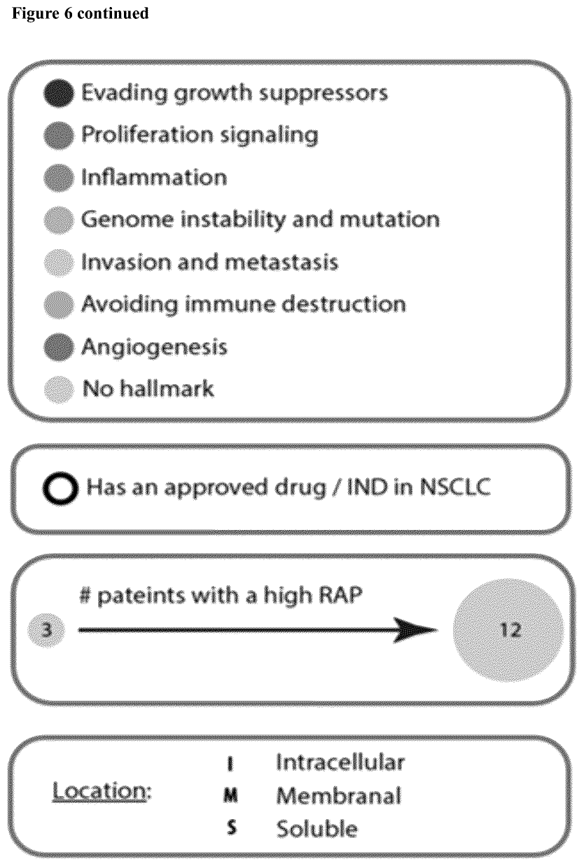

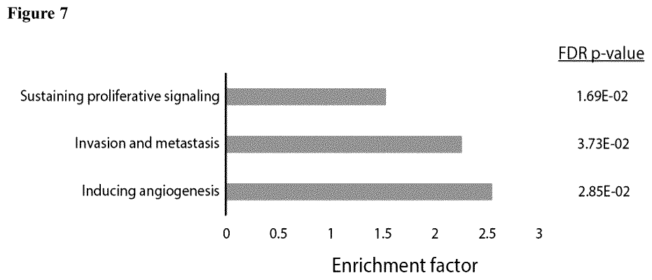

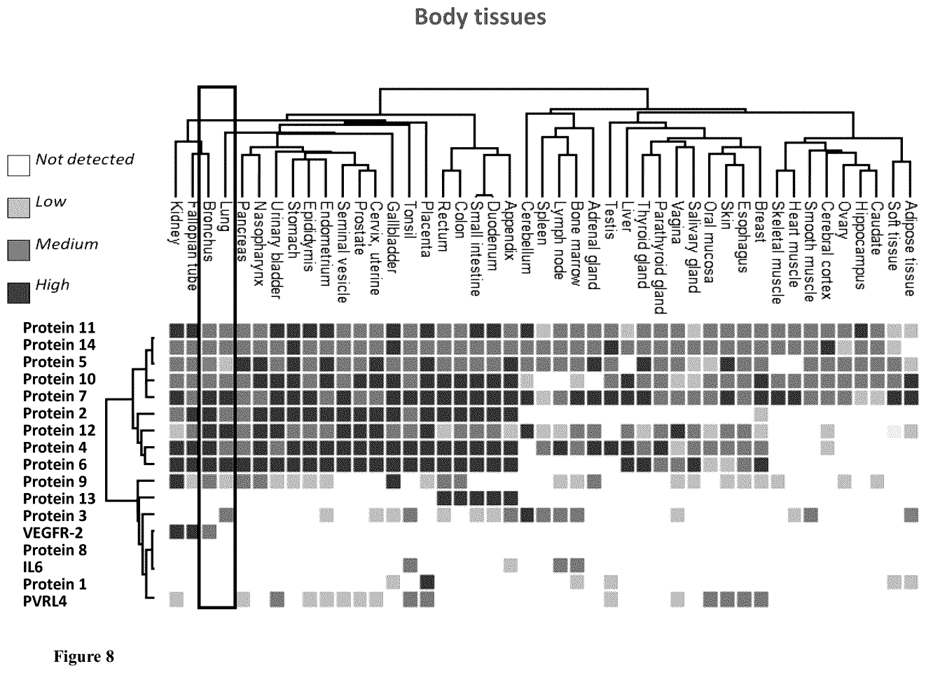

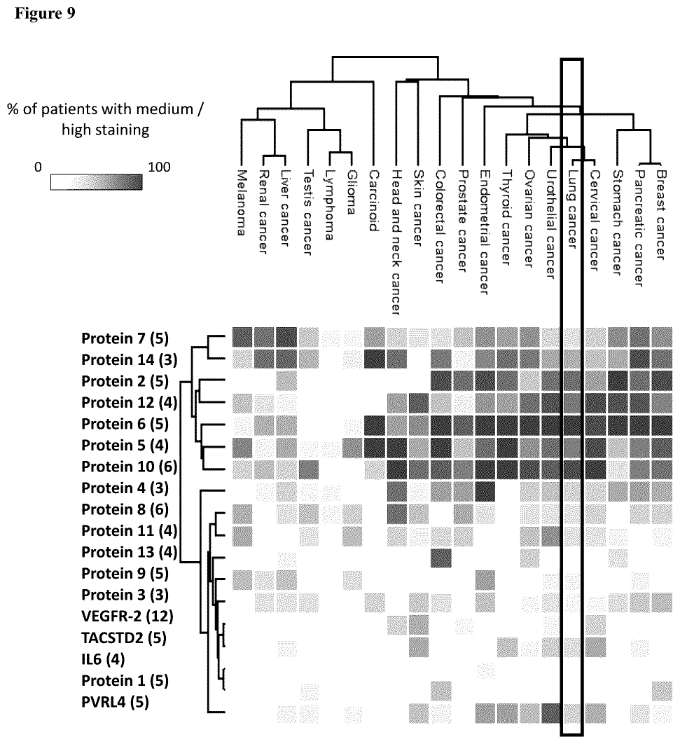

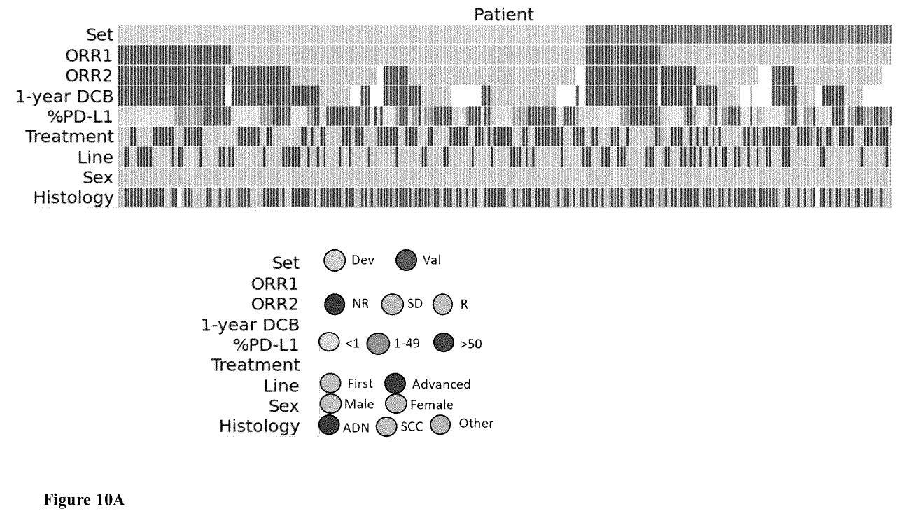

A network analysis revealed that most of the RAPs are functionally associated with each other, and five of them are highly interconnected (FIG. 6). Most proteins are associated with at least one hallmark of cancer, which further implies that these RAPs are indeed associated with resistance to therapy. Several hallmarks of cancer were significantly enriched with the 19 RAPs (FIG. 7). Multiple intracellular and membranal proteins were identified as RAPs (FIG. 6); therefore, an analysis of presumed cell of origin was performed to further understand the results (FIG. 8). Enrichment for lung and bronchus as the cell type of origin was observed. Further, various cancer types were examined for expression of the 19 RAPs and enrichment for lung cancer was also observed (FIG. 9).

### Example 2: Combining RAPs and Clinical Data

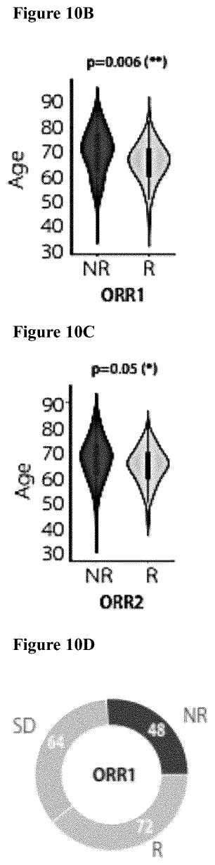

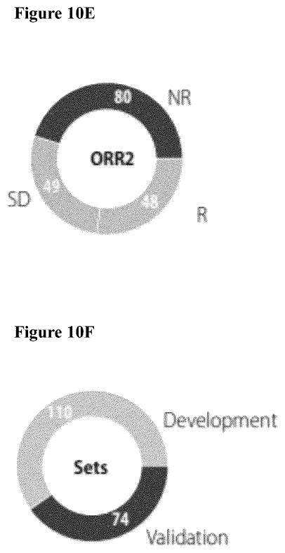

A cohort of 184 NSCLC patients was acquired from which blood samples were obtained prior to the first administration (T0) and after the first (T1) administration with ICI. Protein levels were measured. Response evaluation was based on ORR at three months and six months and durable clinical benefit (DCB) at one year post treatment initiation. Progression free survival (PFS) and overall survival (OS) were also monitored. For 3- and 6-month evaluation, subjects with progressive disease or death were considered non-responders, while subjects with stable disease, minimal remission, partial remission and complete remission were considered responders. DCB was defined as one year of PFS with continued ICI treatment. Cases of ICI treatment stop due to adverse event (but no signs of progression) were treated as responders. Additional clinical information collected throughout the study included: line of treatment (first or advanced), PD-L1 immunostaining (below 1%, between 1-49%, above 50%), age and sex (see FIG. 10A-10F). The presented analysis is based on T0 only. The breakdown of ICIs/therapies used is provided in Table 3.

TABLE 3

| Treatment | Target | Patient count |
|---|---|---|
| Pembrolizumab | PD-1 | 54 |
| Pembrolizumab, Chemo | PD-1 | 86 |
| Nivolumab | PD-1 | 25 |
| Nivolumab, Chemo | PD-1 | 2 |
| Ipilimumab, Nivolumab | CTLA4 | 7 |
| Ipilimumab, Nivolumab, Chemo | CTLA4 | 6 |
| Atezolizumab | PD-L1 | 1 |
| Atezolizumab, Chemo, targeted therapy | PD-L1 | 1 |
| Durvalumab | PD-L1 | 1 |
| Durvalumab, Chemo | PD-L1 | 1 |

The cohort was divided into a development set (60% of the subjects) and a validation set (40% of the subjects). The development set was further divided into training set and test set. The models were trained on the training set and predictions were generated for a subset of patients not seen by the models during training (i.e., test sets). The division of the development set into training and test set was performed multiple times (each time for training the model on a different subset of the development set and performing predictions on the remaining patients. i.e., the training and test sets were mixed and remixed and tens of iterations were run to test that a model/classifier was effective across the entire development set) in order to generate a stable prediction for all patients in the development set. The prediction quality was then quantified by calculating the ROC AUC for the patients included in the development set. The validation set was used only at the very end of the analysis to validate the functionality of the final classifier. This division was performed multiple times,

Models were generated based on response evaluation at three time-points: three months, six months, and a year after treatment onset. All 184 patients were evaluated at the three-month time point, 177 were evaluated at six months and 146 were evaluated at 1 year. Resistance increased over time. 26% of the subject were non-responders at three months, 45% were non-responders at six months and 74% were non-responders at 1 year. These ratios were similar between the development and validation sets.

During model generation based on the development set, the development set was randomly divided into a training and a test sets 60 times. On each iteration, the top candidate proteins were selected using the Kolmogorov-Smirnov test that defines for each protein how much it differentiates between responders or non-responders. For each selected protein, a single protein XGBoost model (SP model) was generated based on the training set and predictions were made for the test set. A protein was defined as a RAP for a specific patient if the predicted resistance probability (i.e., the resistance score) was above a predefined threshold, and the average of all the iterations was used for each patient. A uniform threshold was assigned for all models, in order to handle class imbalance. Different thresholds were defined for each time point (e.g., three months threshold=0.25, six-month threshold=0.42, one year threshold=0.45). For each patient, the number of proteins for which the model score exceeded a defined threshold (i.e., the number of RAPs) was calculated.

Merely looking at the number of RAPs was predictive with this cohort. However, a predictor model was created that could also integrate clinical data. The presented clinical classifier used the number of RAPs, the line of treatment (was the ICI the first line of treatment or an advanced line), the subject's age and the percent of PD-L1 staining in the tumor (below 1% of cells positive, between 1-49%, or above 50%) as the inputs. The classifier then produced a final resistance score between 0 and 1, in which 0 was most similar to responders and 1 was most similar to non-responders. Subjects with a score above a predetermined threshold were predicted to be non-responders. Similarly, a response score, which is 1-resistance score, was also calculated. For the response score, a subject with a score above a predetermined threshold was predicted to be a responder.

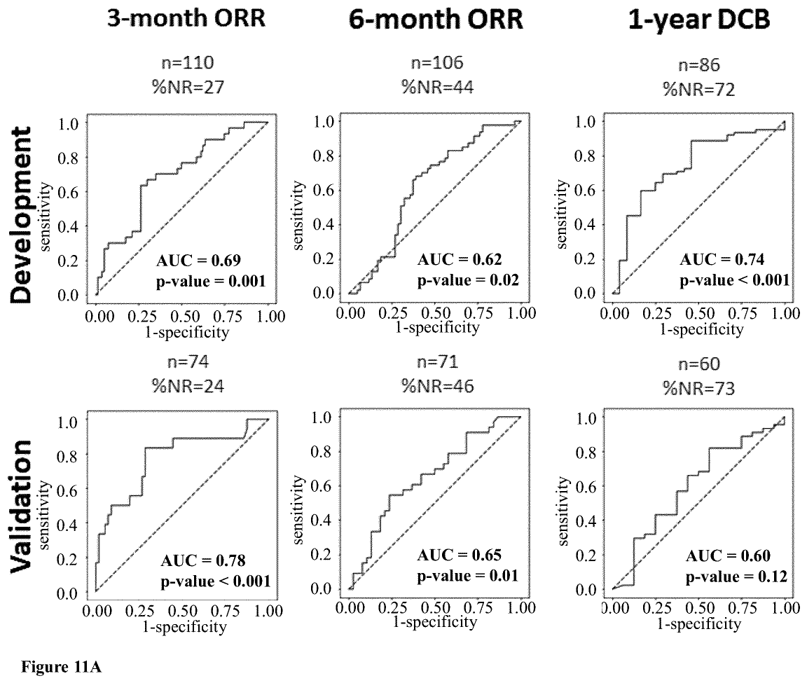

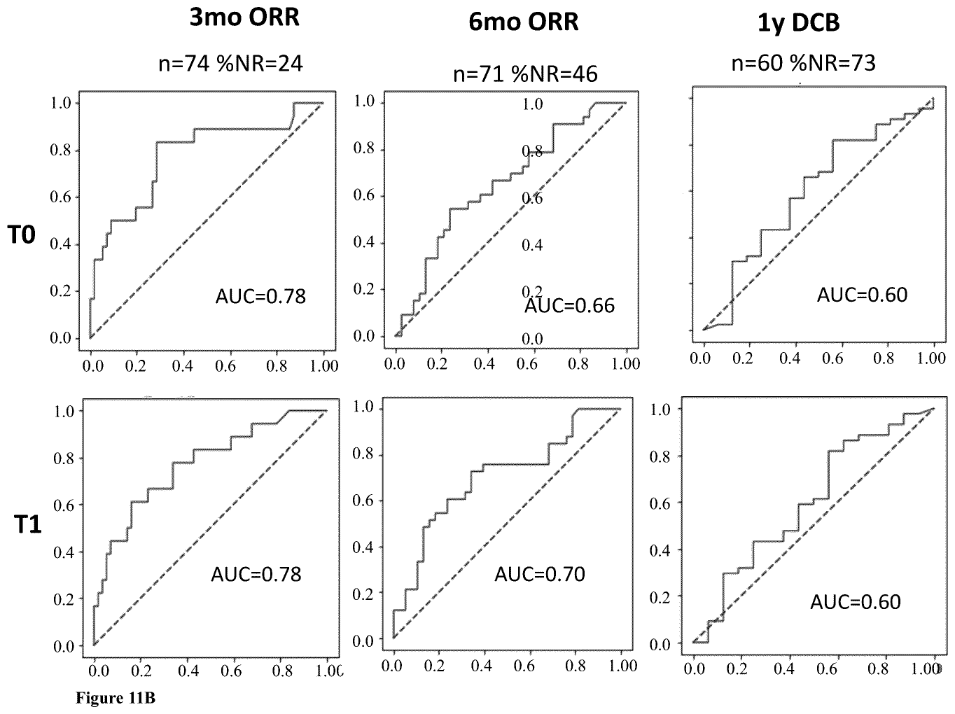

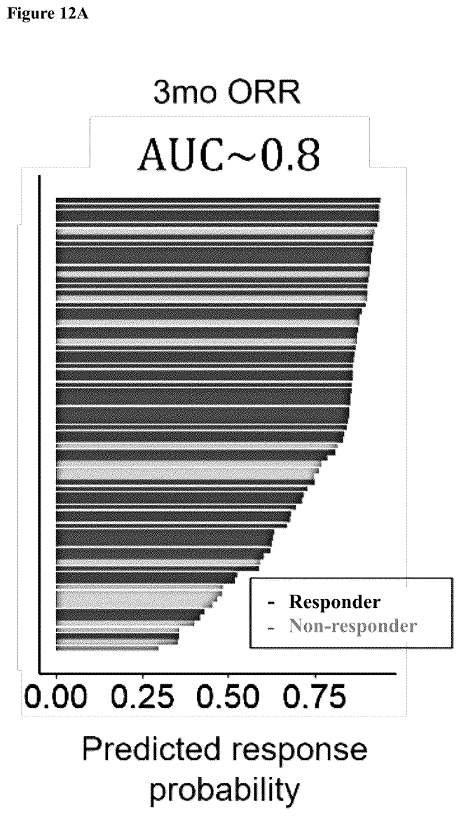

In order to test the performance of the classification model, a ROC AUC was calculated using the final resistance score together with actual response. The ROC AUC was calculated separately for 3-months ORR, 6-months ORR and 1-year DCB for both T0 and T1. The results are summarized in FIG. 11A. The classifier was found to be predictive at all time points and for both development and validation sets. A similar analysis showed that the classifier was found to be predictive at all time points also for the T1 data (FIG. 11B).

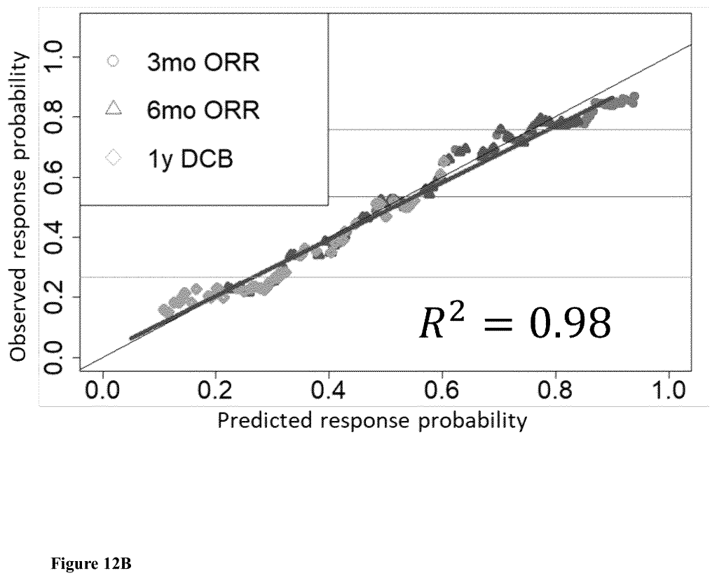

Further to checking the performance of the classification model, the correlation between the predicted response probability (response score) assigned by the classification model to each patient and the observed response probability was also examined. For this purpose, for each value of response score S₀, the observed response probability is given by the fraction of responders among patients that were assigned a response score within the range S₀±0.1. The choice of an interval of ±0.1 is arbitrary and reflects the validation set size; within a larger validation set the interval can be further reduced. The agreement between the predicted response score and the actual response probability was quantified by the goodness of fit R\hat{ }2. The goodness of fit for all 3 timepoints (3 months ORR, 6 months ORR and 1 year DCB) was R\hat{ }2=0.98 for time point T0 (FIG. 12A-12B).

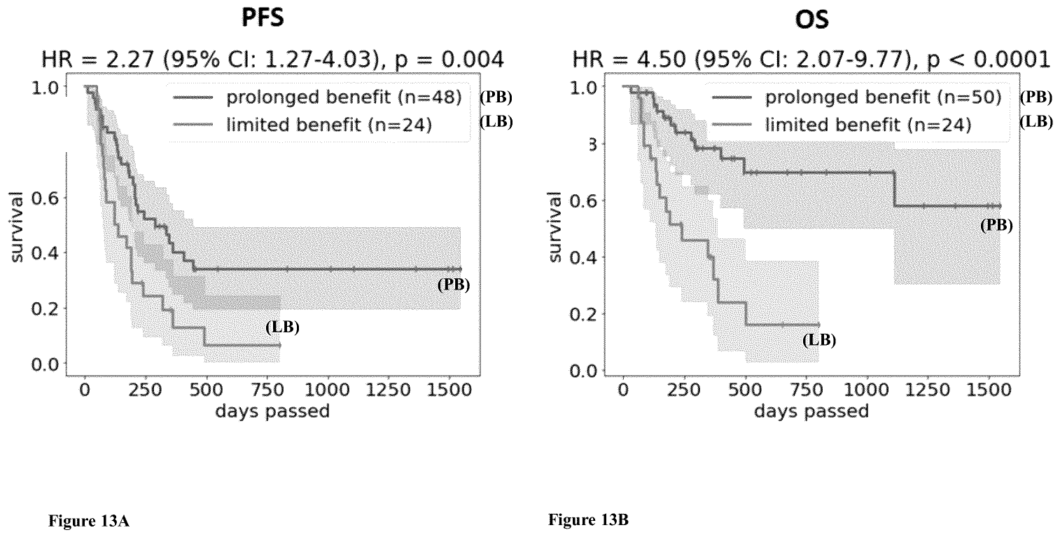

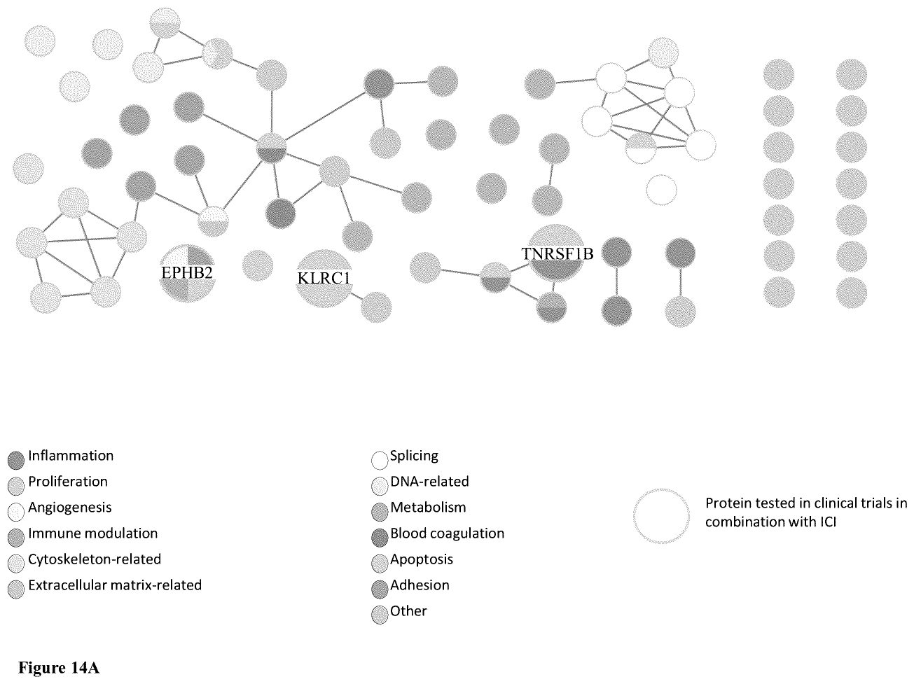

Patients within the validation set were stratified to prolonged benefit and limited benefit populations, where the stratification was based on the predicted 3-month response score. In survival analysis the stratification quality was measured by the hazard ratio (HR), which gives the ratio of probability for event per time unit within the two population. For example, HR of 4 in overall survival (OS) means that the probability for a death event per unit time among the limited benefit population is 4 times the probability per unit time among the prolong benefit population. The HR in the validation set was 2.27, p<0.004, for PFS (FIG. 13A) and 4.50, p<0.0001, for OS (FIG. 13B).

This validation experiment demonstrates that the classifier that incorporates clinical data and RAP number is highly predictive of patient response.

### Functional Network Analysis of RAPs

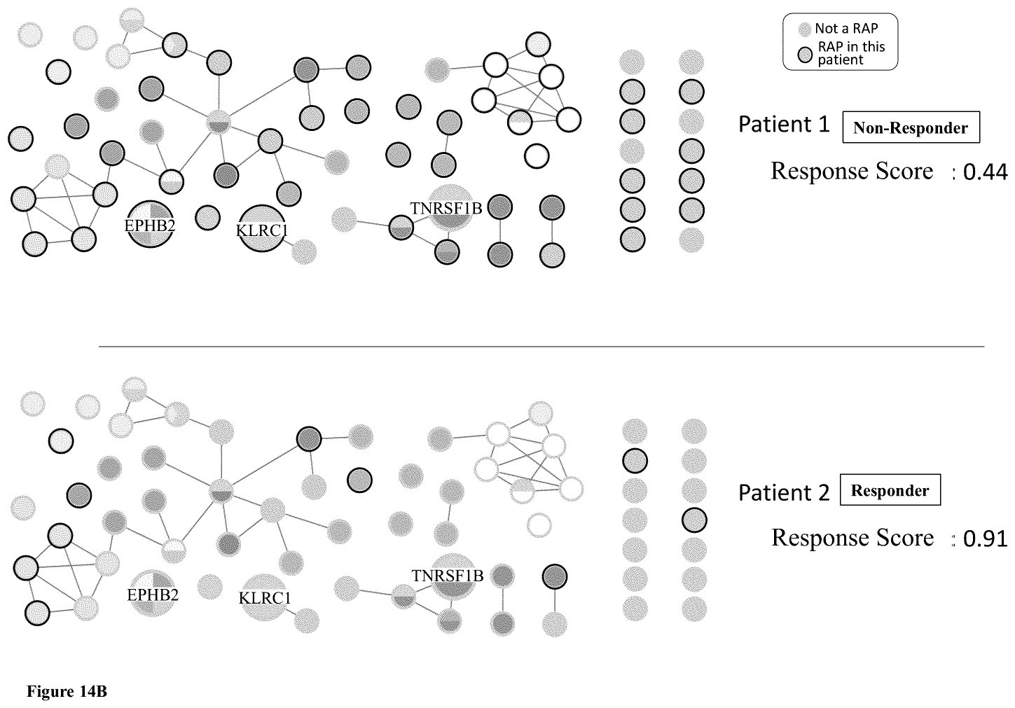

The RAP-based analysis is further used as a basis for the generation of resistance maps (FIG. 14A). The resistance map displays both the interactions between RAPs and the RAP functions. For this purpose, a RAP was defined when a protein was selected in at least 10 model iterations in one or more patients (during the RAP calculations, the model runs 60 iterations, and the number that a given protein is selected for the model is recorded), resulting in a total of 73 RAPs in the current cohort of patients. Each node represents a RAP, and the edge between nodes indicates a functional relation. Nodes with a larger size indicate investigational new drugs (INDs) in combination with immunotherapy. The nodes are colored based on the protein function. The map shows multiple interactions between different RAPs, while the RAPs are involved in different functional processes that may be relevant for resistance to therapy, such as splicing, immune modulation, angiogenesis and cell proliferation. A patient-specific map can be generated based on the patient's RAPs, which aids in 1) mapping resistance mechanisms in the individual patient and 2) identifying targeted treatments that counteract resistance. Two examples of patients in the cohort are illustrated in FIG. 14B. In these examples, a non-responder had 44 RAPs and a response probability score of 0.44 (which corresponds to a resistance score of 0.56 which is above the predetermined threshold of 0.2 for non-response). This patient had RAPs from multiple functional groups, but DNA-related RAPs were not present in this patient. The second subject was a responder with 10 RAPs, below the predetermined threshold. These RAPs were mainly related to the cytoskeleton. This patient had a high response probability of 0.91 (which corresponds to a resistance score of 0.09 which is below the predetermined threshold of 0.2.

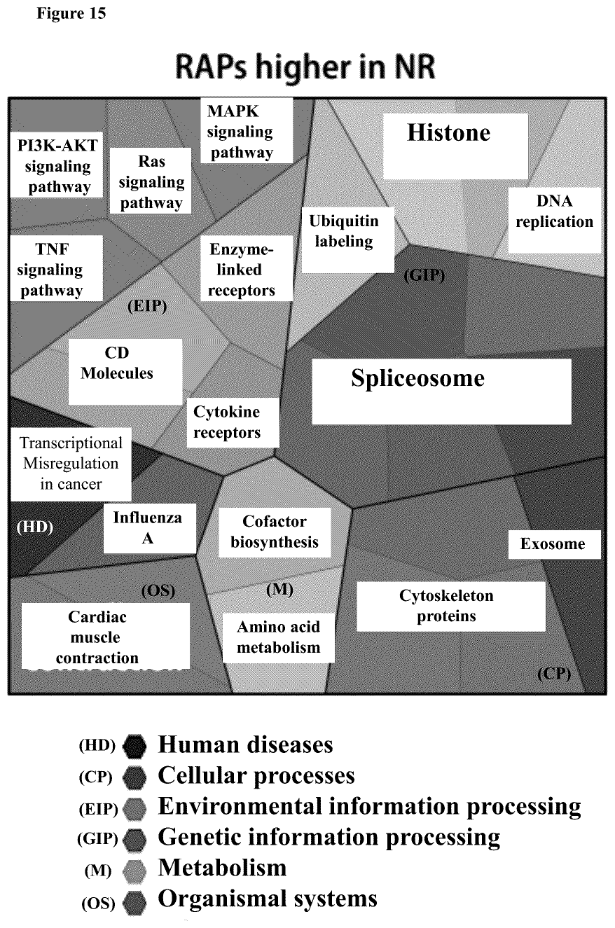

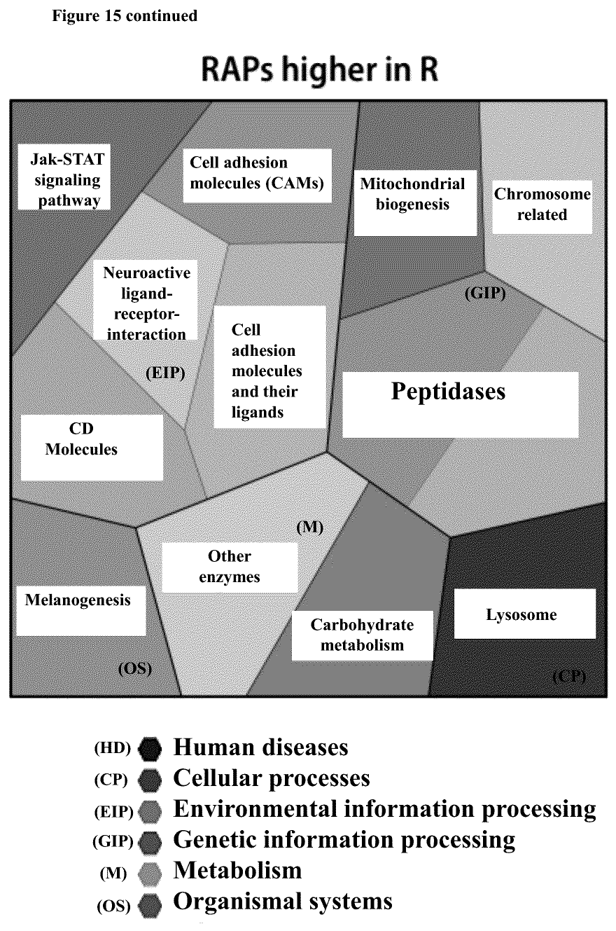

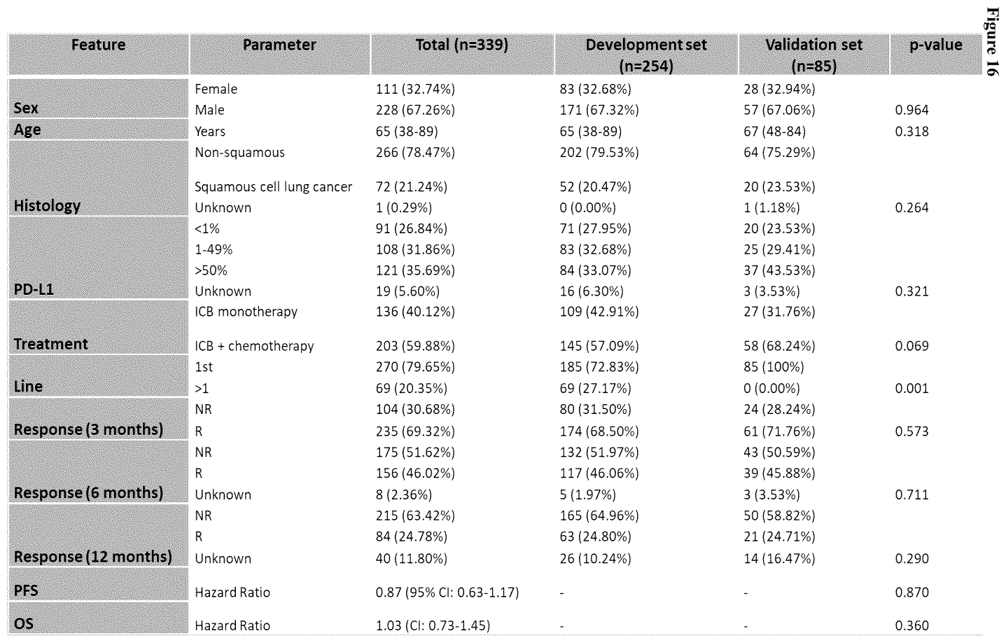

Further examination of the patient RAPs shows functional differences between RAPs with higher representation in each response group (FIG. 15). While non-responder RAPs are involved in splicing, signaling and cytoskeleton-related processes, the responder RAPs are mainly involved in proteolysis and cell adhesion. Interestingly, RAPs higher in the responder group includes 2 peptidases that may be involved in antigen presentation, thereby promoting response to therapy. In order to convert non-responders to responders a RAP is selected for which there is a known therapeutic agent. The agent is selected such that it modulates the RAP to alter pathway function to more closely approximate pathway function in responders. If therapeutics that target the RAPs are unavailable or undesirable a therapeutic agent that modulates the pathway containing the RAP is selected. The selected agent must modulate the pathway containing the RAP to alter pathway function so that it more closely approximates pathway function in responders. The therapeutic agent is used to convert non-responders to responders or as a combination treatment with the ICI.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

### Example 3: Validation of the RAP-Based Model

To validate the previously described RAP-based models, a larger cohort of NSCLC patients was used. Blood plasma samples and clinical data from 339 ICI-treated NSCLC patients were collected. Pre-treatment plasma samples were profiled by a protein assay that quantifies approximately 7000 proteins in a single plasma sample.

Patient clinical parameters are presented in FIG. 16A. The median age was 65. Approximately a third of the patients were female. The majority of patients (78.47%) had non-squamous cell carcinoma (mostly adenocarcinoma) and 21.24% of the patients had squamous cell carcinoma. Patients were either treated with immune checkpoint blockade (ICB)-chemotherapy combinations (59.88%) or ICB monotherapy (40.12%). There was an approximately equal distribution of patients with negative (<1%), low (1-49%) and PD-L1-high (>50%) expression of PD-L1. The PD-L1-high group was the largest (36%).

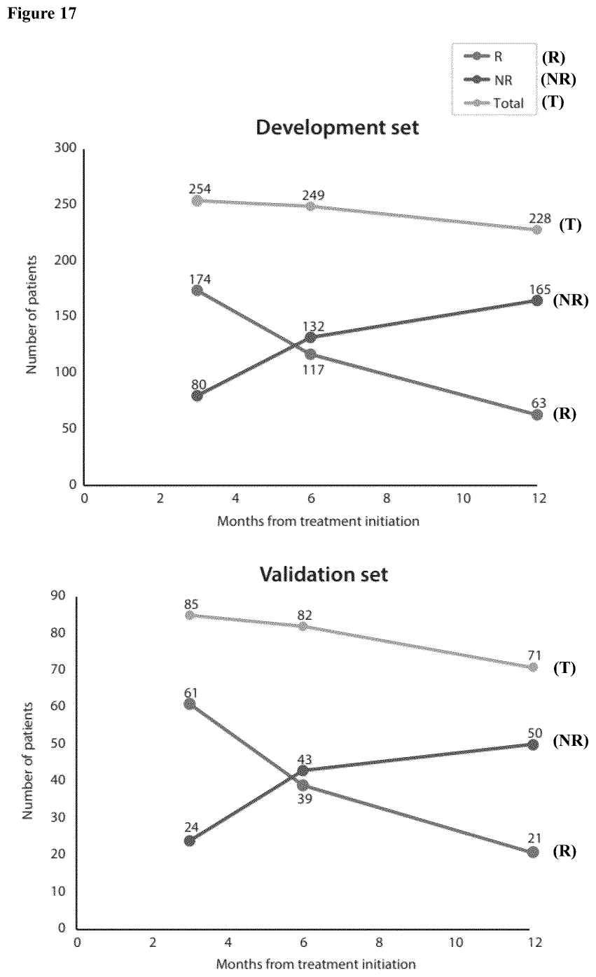

Clinical response to treatment was assessed at 3, 6 and 12 months after commencement of treatment and at each time point, patients were classified as responders or non-responders. At the 3- and 6-month time points, patients with complete remission, partial remission or stable disease were classified as responders and patients with progressive disease were classified as non-responders. At 12 months, patients displaying durable clinical benefit (defined as absence of progressive disease for at least 1 year after starting treatment) were classified as responders, and all other patients were classified as non-responders. Based on these criteria, 69.32%, 46.02% and 24.78% of the patients were classified as responders at 3, 6 and 12 months, respectively (FIG. 16). The cohort size varied between time points due to patient death (FIG. 17). The dataset included 339, 331 and 299 patients for the 3, 6- and 12-month time points, respectively.

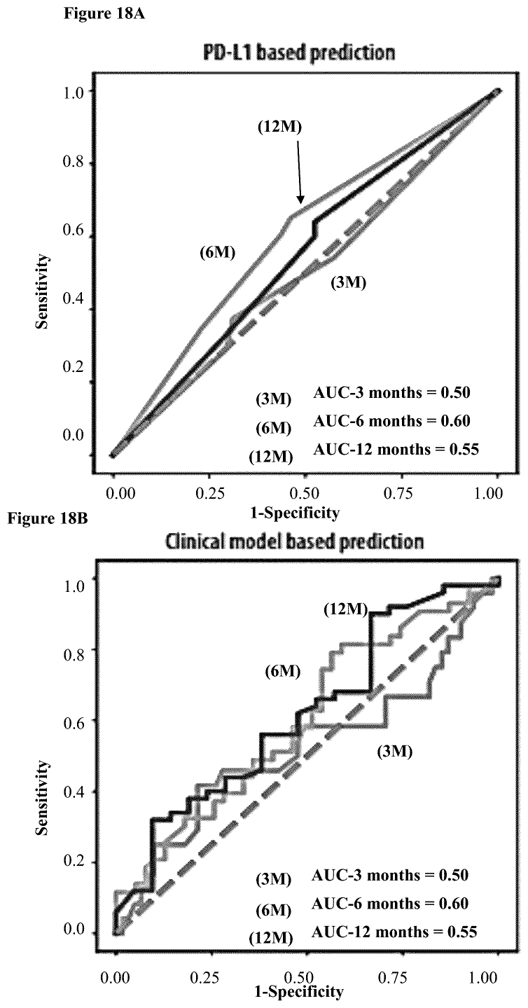

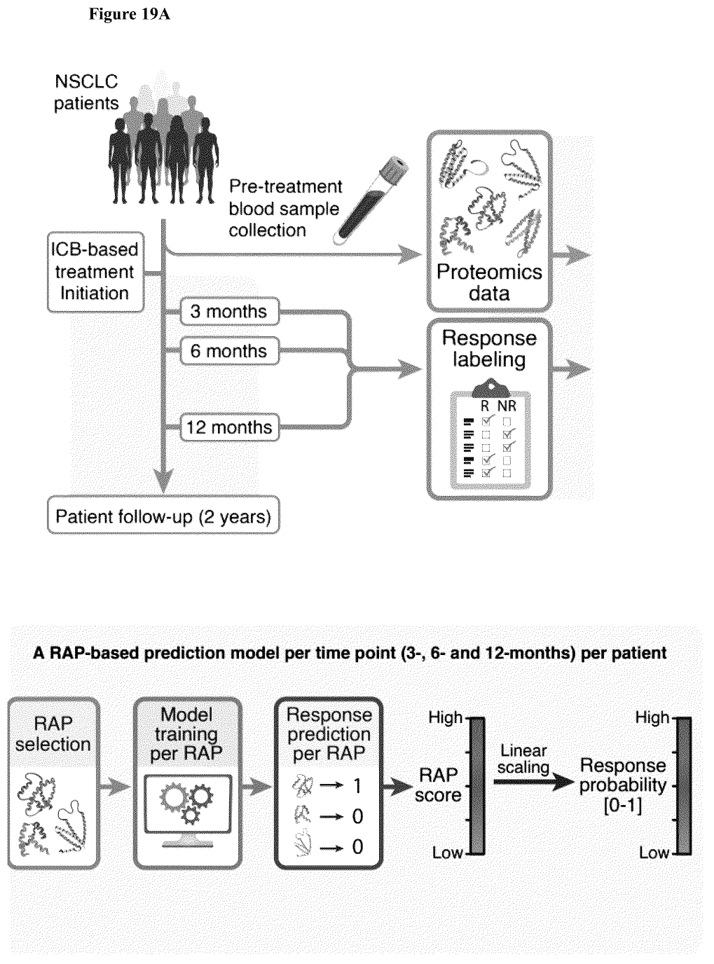

Even though PD-L1 expression correlated with response at the 6- and 12-month time points, response prediction using only this parameter at each of the 3 time points was poor, with area under the curve (AUC) of the receiver operating characteristics (ROC) plot of 0.5, 0.6 and 0.55 at 3, 6 and 12 months, respectively (FIG. 18A).

Next it was asked whether integrating additional clinical parameters would improve the predictive capability of the PD-L1 biomarker. Three clinical parameters are known to correlate with response, namely, patient age, patient sex, and line of treatment. Accordingly, a predictive model based on PD-L1, age, sex and treatment line (the ‘clinical model’) was developed. The clinical model displayed only a minor improvement in response prediction capability compared to PD-L1 alone, with AUCs of 0.52, 0.6 and 0.62 for 3, 6, and 12 months, respectively (FIG. 18B). Further improvements in predictive performance are required.

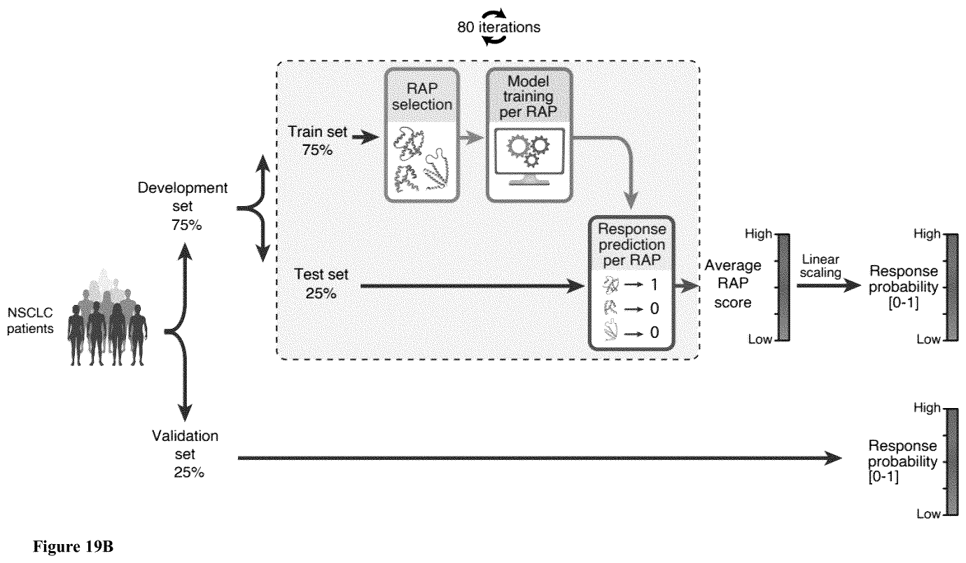

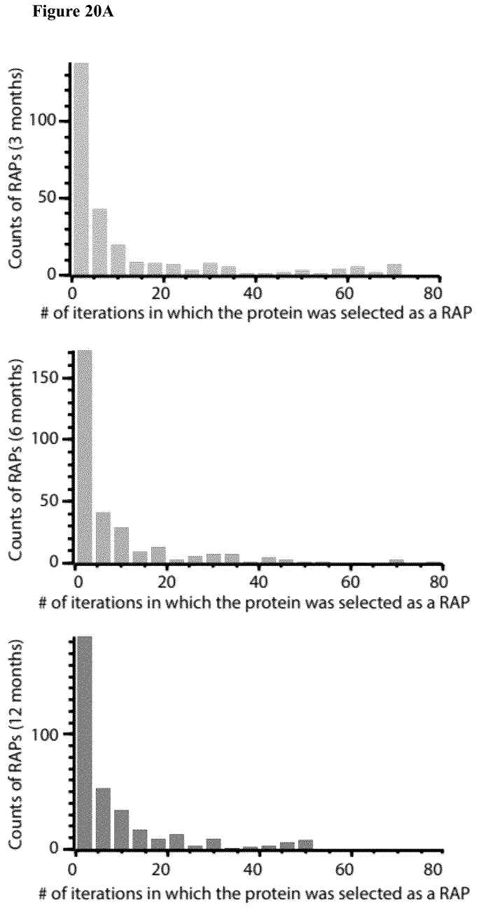

Aiming to develop a more robust predictive model, an additive model where the output is a sum of individual features associated with therapy response was designed. Since each feature on its own has a minor effect on the final output, the effects of any false discoveries are minimized, and model stability is maintained, thereby potentially mitigating the effects of significant heterogeneity between patients and the large number of features in a comparatively small cohort. The model is based on a set of proteins that display differential plasma levels in responder and non-responder populations, as determined by a statistical test (RAPs). Such proteins serve as potential indicators of treatment response depending on their plasma level in the individual patient. Specifically, for a given patient, a ML-based model that was trained on responder and non-responder populations infers a prediction of ‘active RAP or ‘non-active RAP from the plasma level of each one of the patient's potential RAPs within the entire RAP set. In this way, the patient is assigned a personalized RAP profile. The sum of the number of personal RAPs reflects the patient's likelihood of responding to treatment. Patients displaying numerous ‘active predictions in the RAP set (and thus many personal RAPs) are more likely not to respond to treatment, whereas patients with numerous ‘not-active’ predictions in the RAP set (and thus few personal RAPs) are more likely to respond to treatment. Similarly, the ML-based model actually provides an activity score for each RAP. These scores can be combined (summed) to produce a total RAP score, which can in turn be used to predict the patient's response (more activity from the RAPs a higher likelihood of non-responsiveness). Three RAP-based models were developed, one for each of the three response assessment time points. The models were developed following the same workflow, where response labelling for the 3, 6- or 12-month time points, together with protein expression data and patient sex, were used as input for determining personal RAPs and response probability (as determined by total RAP score) (FIG. 19A). The cohort of patients was divided into a development set (75% of the patient cohort; n=254) and a validation set (25% of the patient cohort; n=85). The development set was further divided into train and test sets consisting of 75% and 25% of the development set patients, respectively (FIG. 19B). Proteins displaying statistically significant differences in their plasma level distributions between responder and non-responder populations were identified in the train set, and the 50 most significant proteins were identified as the general RAP set. For each RAP, a ML algorithm was trained with two features, namely, RAP plasma expression level and patient sex, to develop a predictive model for RAP activity/expression based on the train set. Activity predictions (is the protein an active/expressed RAP in a given subject) were then generated per RAP for each patient in the test set, yielding a prediction of ‘active’ or ‘non-active’ for each single RAP. The 3-step process (i.e., RAP selection, model training and activity prediction) was repeated 80 times, each time with a random division of the patients into train and test sets. In each iteration, the activity scores across the 50 selected RAPs were summed up per patient to yield a total RAP score. After 80 iterations, the total RAP scores were averaged per patient and linearly scaled to values between 0 and 1. The final output of the model was transformed to response probability—a clinically oriented metric reflecting the patient's likelihood of responding to treatment. (Similarly, the total number of active RAPs could be used to make a response prediction (as described supra).

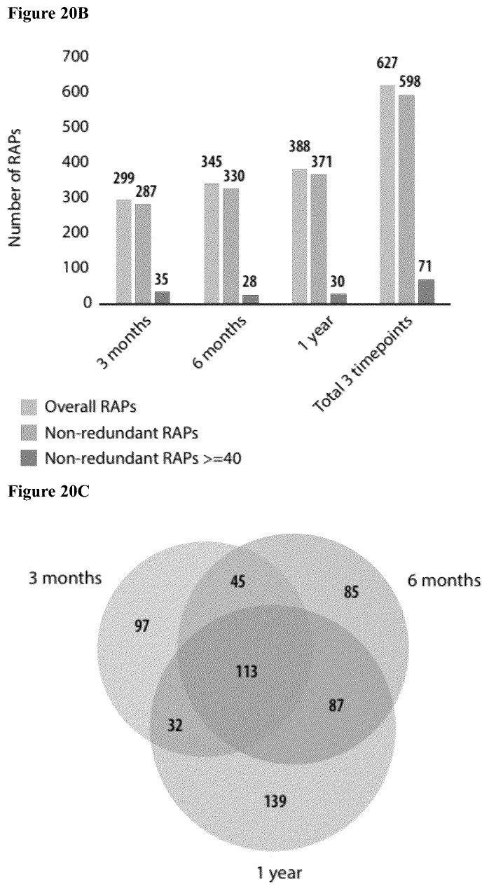

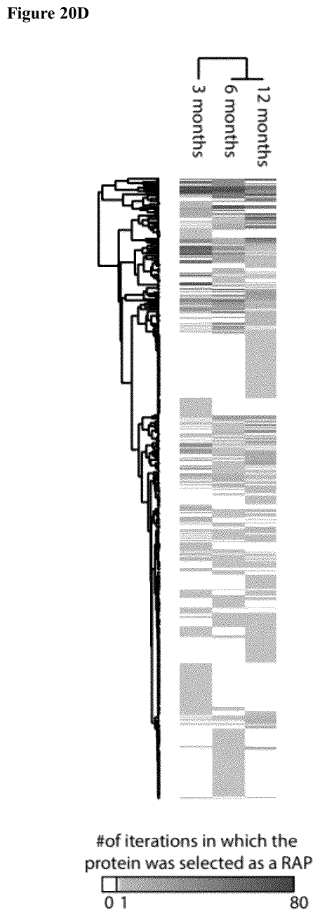

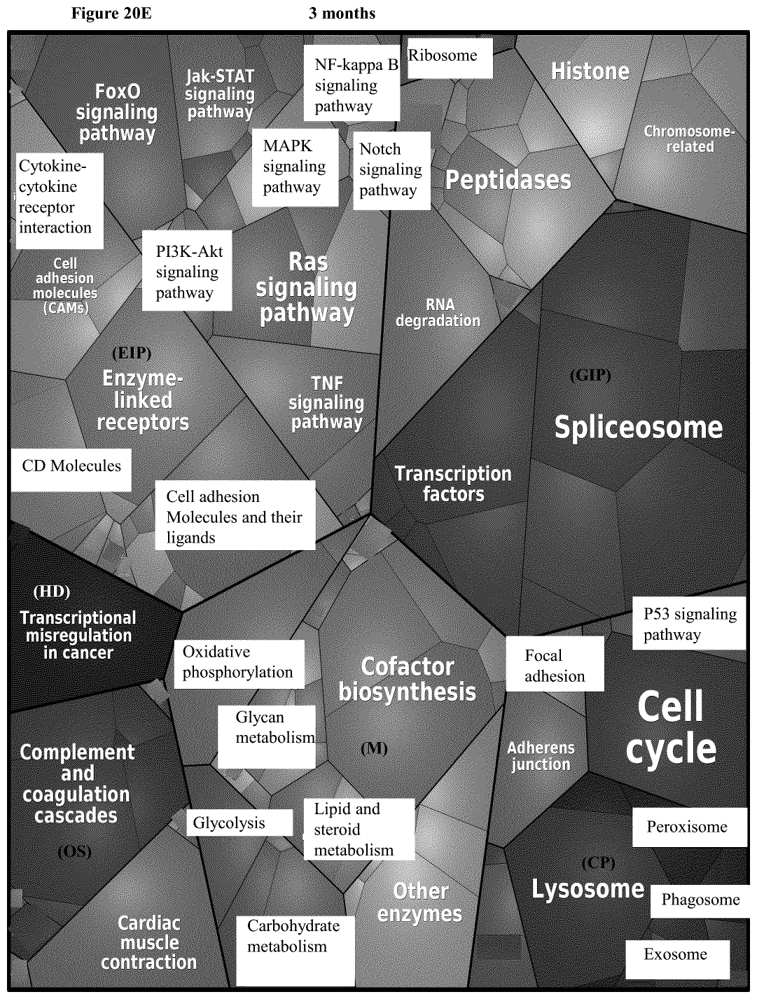

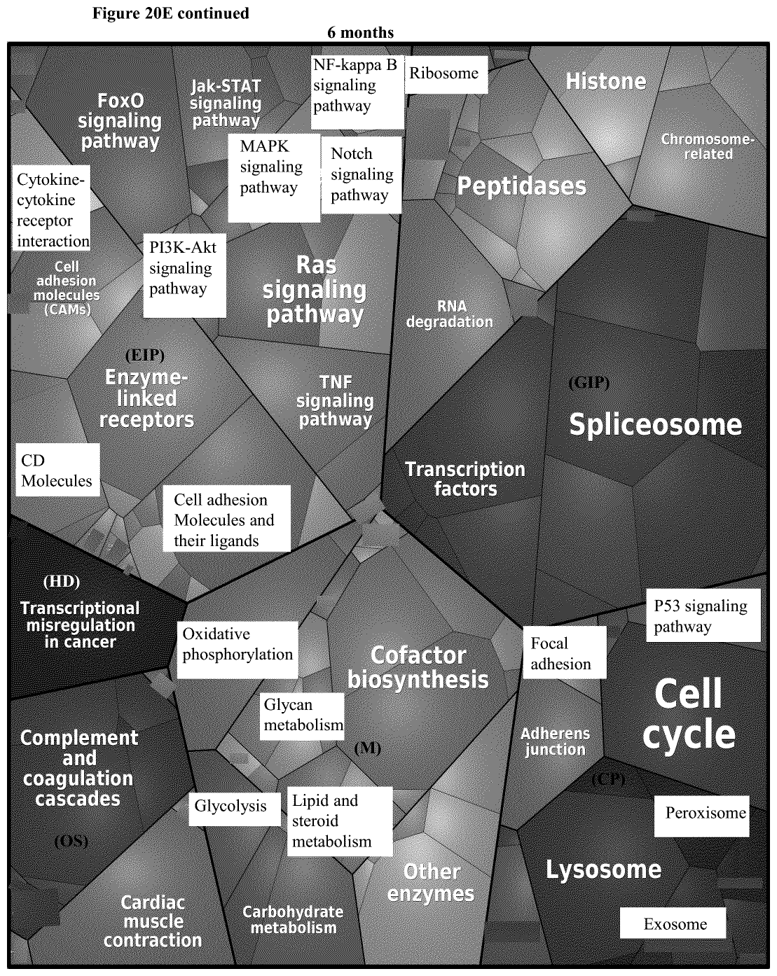

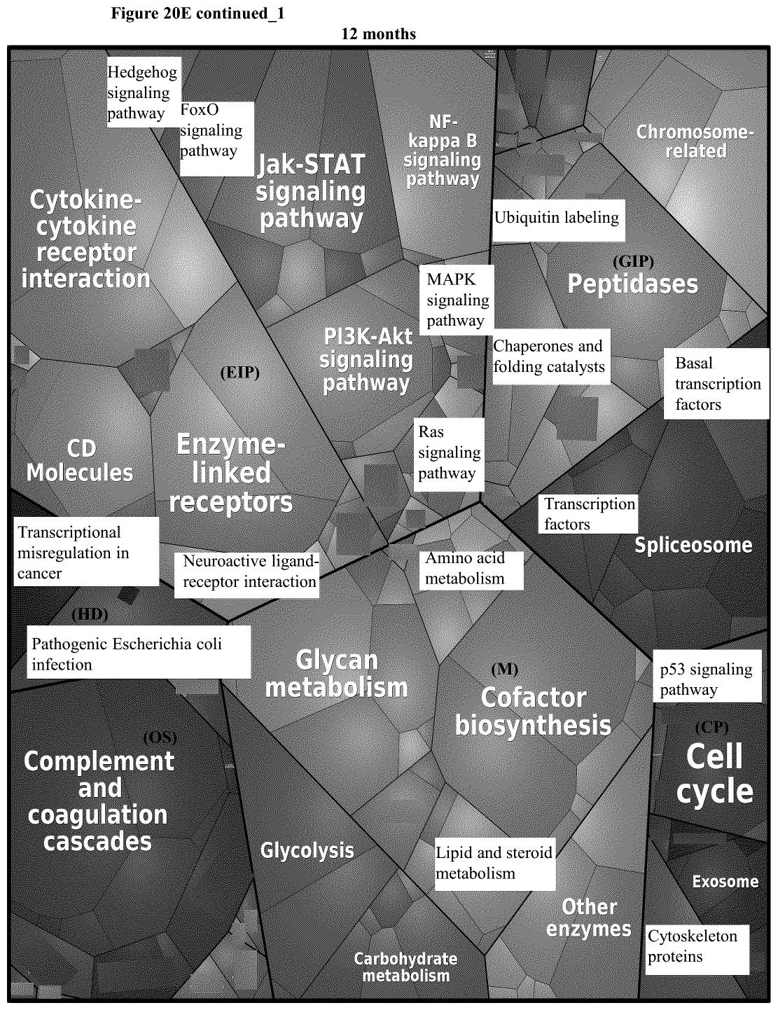

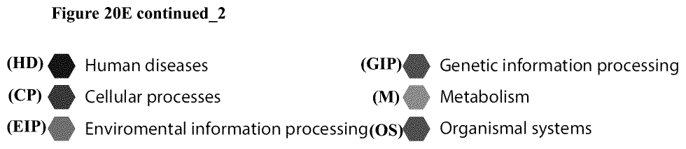

Using this method randomly mixing the patients between train and test sets 80 times, and in which at each time 50 RAPs were selected from the train set, the same RAPs could be selected several times overall (FIG. 20A). Out of a total of 287, 330 and 371 RAPs selected for the 3, 6- and 12-month time points, respectively, approximately 30 RAPs were selected in at least 50% of the iterations (>40 times) per time point (FIG. 20B). Across the 3 time points, a total of 598 RAPs were selected, out of which 113 RAPs were common to all 3 time points, while 97, 85 and 139 RAPs were unique to the 3, 6- and 12-month time points, respectively (FIG. 20C). Notably, numerous RAPs were selected multiple times across the three time points (FIG. 20D). Biological processes associated with RAPs across all time points include splicing, complement and coagulation cascades, and peptidase activity, as well as and multiple signaling cascades (FIG. 20E).

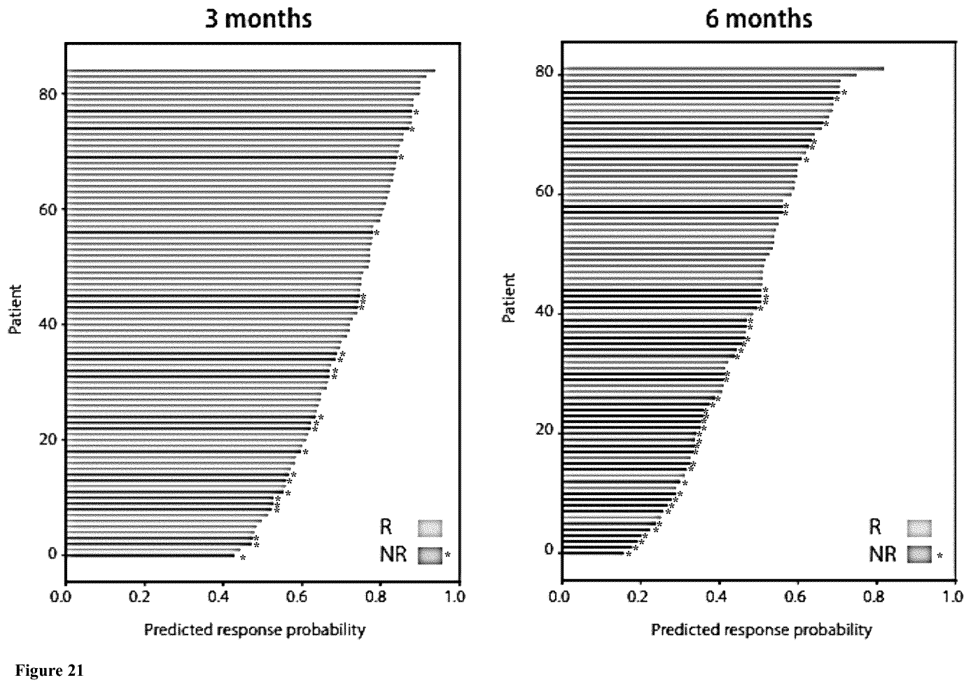

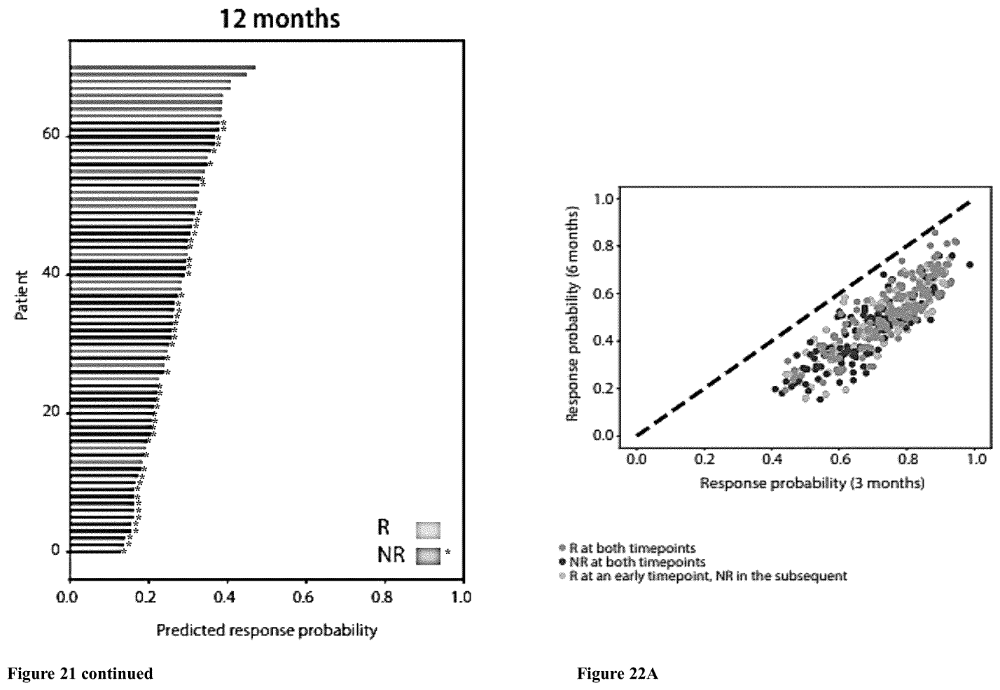

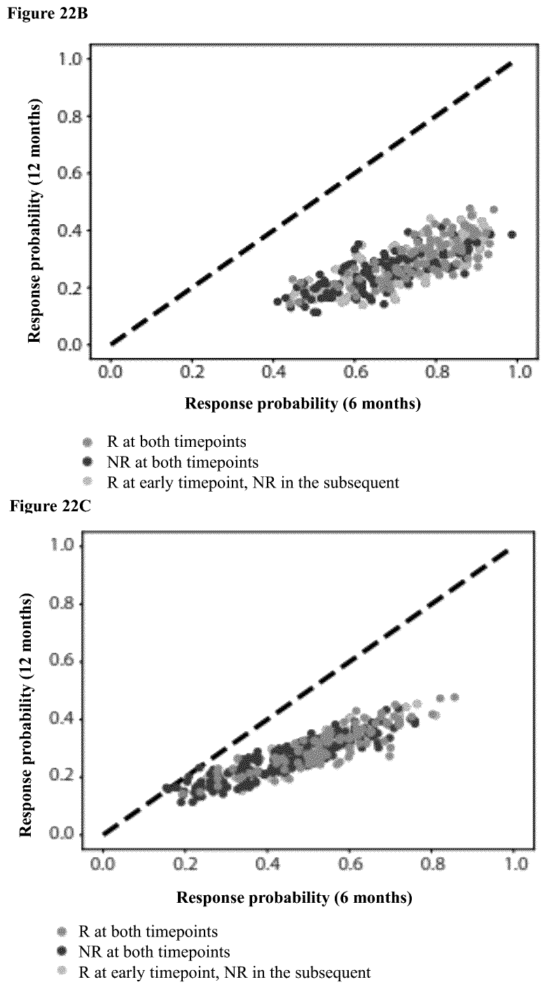

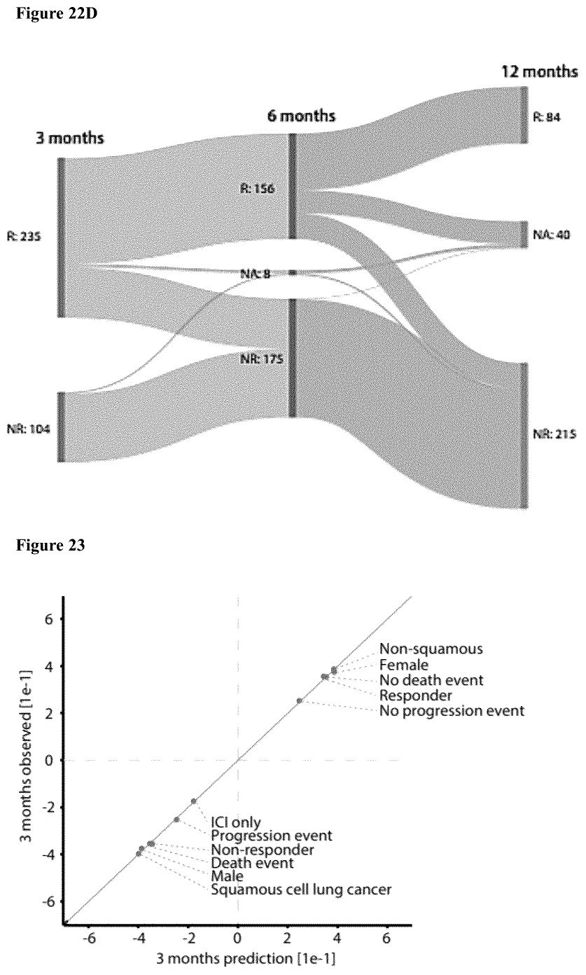

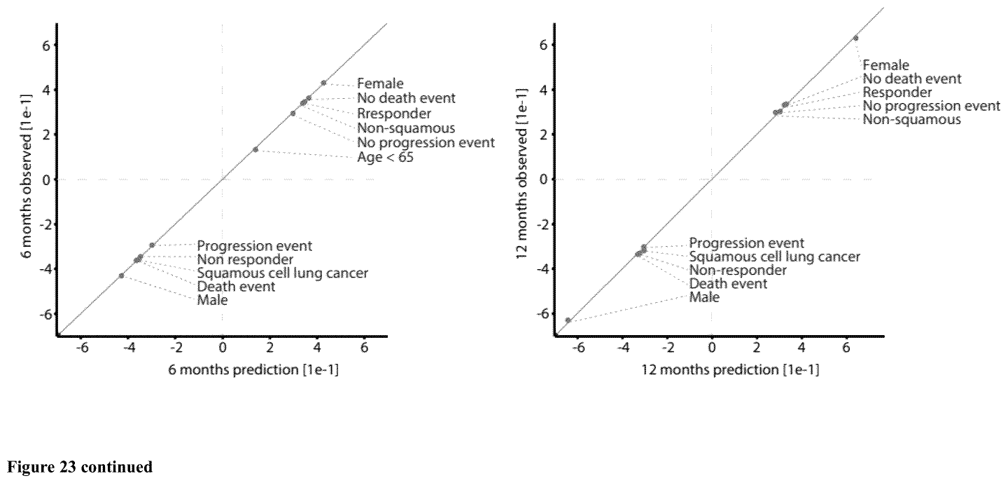

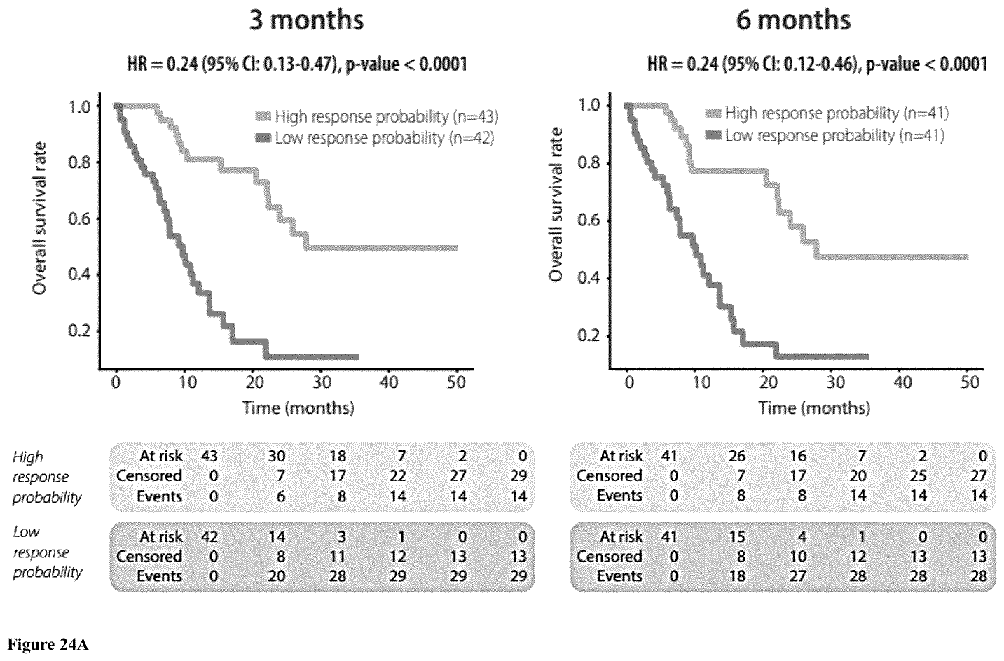

After model development, the RAP-based models for each time point were tested on the independent validation set (25% of the patient cohort; n=85) that was not used during model training. Per time point, response probabilities were determined for each patient in the validation set. The range of the response probability distribution was different for each time point, with a decrease in the median response probability over time (FIG. 16A; FIG. 6). In addition, the response probabilities of all patients decreased from one time point to any subsequent time point (FIG. 22A-22C), in agreement with the decreased observed response rate over time (FIG. 22D). Notably, observed non-responders clustered at the lower range of predicted response probabilities for all 3 time points, indicating that the models have high predictive power (FIG. 21). Moreover, an enrichment analysis based on response probabilities (2D enrichment test; False discovery rate <0.05) showed that, at all three time points, high response probabilities were significantly enriched with responders, females, patients with non-squamous cell carcinoma, and patients with no progressive disease or death events. On the other hand, low response probabilities were significantly enriched with non-responders, males, patients with squamous cell carcinoma and patients with progressive disease or death events (FIG. 23).

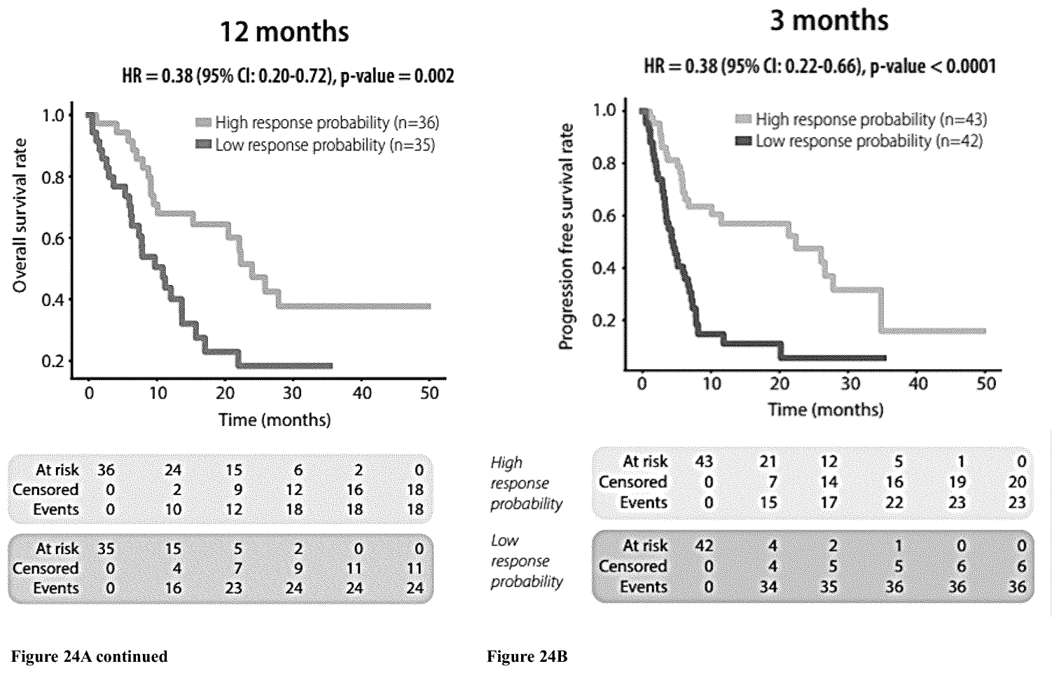

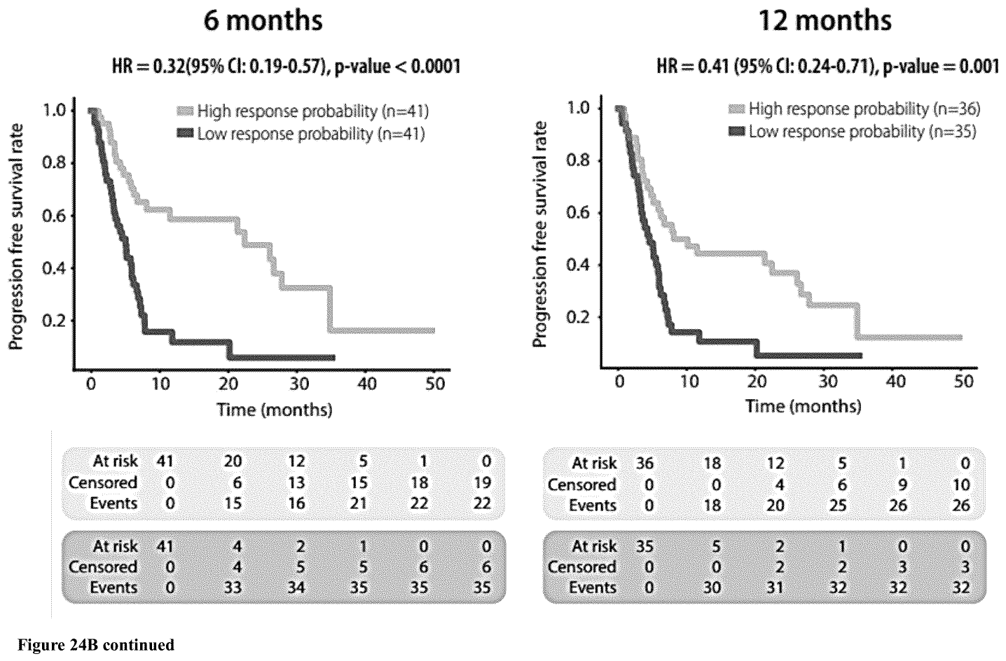

Using the median response probability as a threshold, the patients were classified into high or low response probability groups, where patients with a predicted response probability above or below the median were assigned to the high or low response probability groups, respectively. A Cox regression analysis demonstrated that patients in the high response probability group achieved significantly longer overall survival than patients in the low response probability group across the 3 time points (FIG. 24A, Hazard Ratio, HR=0.24-0.38). Similar results were obtained for progression-free survival (PFS; FIG. 24B, HR=0.32-0.41). These findings demonstrate that the RAP-based models successfully classify survival outcomes in ICB-treated NSCLC patients.

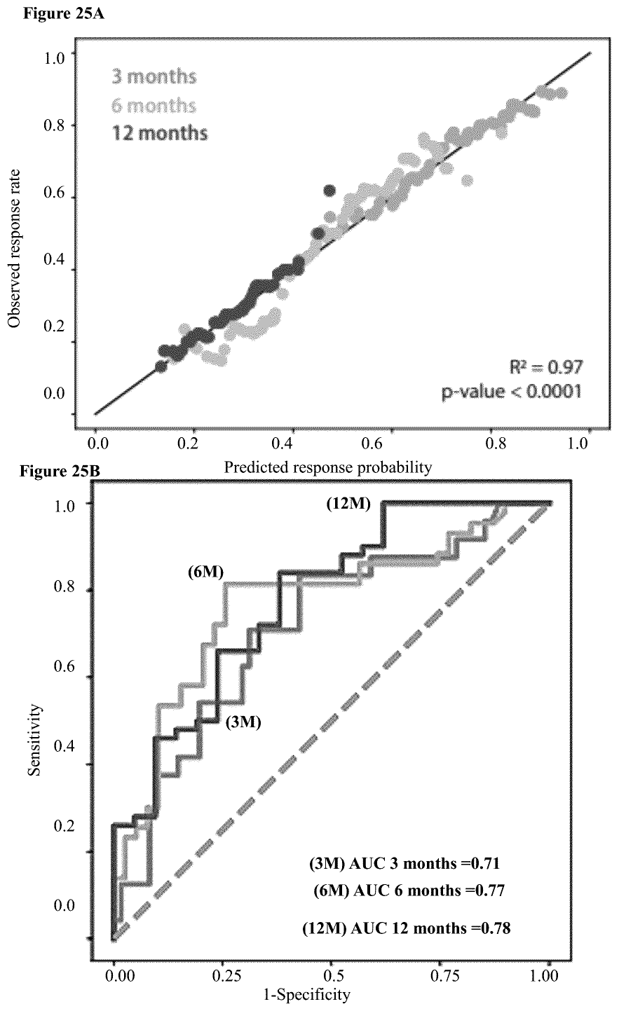

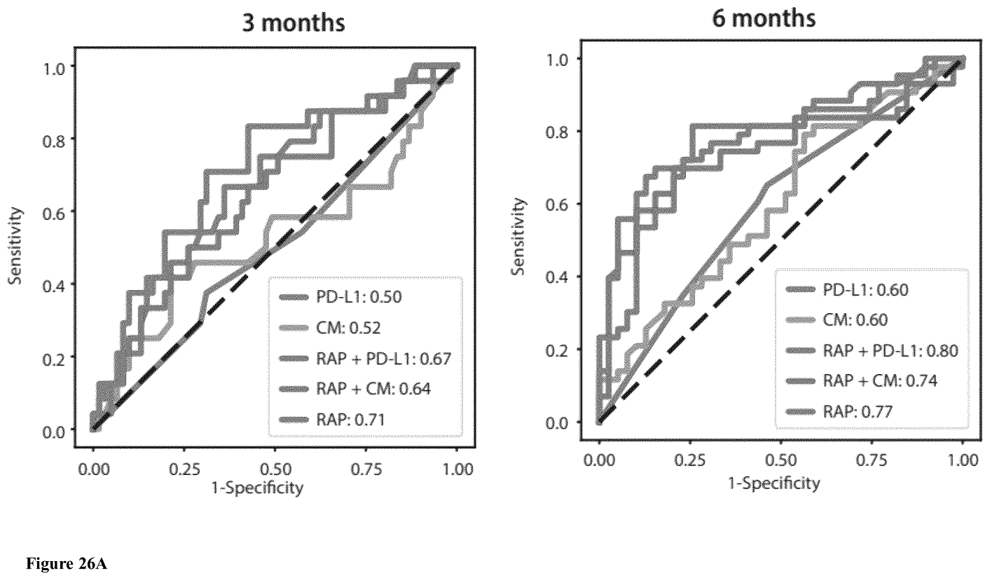

To further test model accuracy, predicted response probability was compared to the observed response rate, where the latter refers to the proportion of observed responders within the group of patients assigned a similar response probability (i.e., response probability ±0.15). Linear regression analysis demonstrated a high goodness of fit (R²=0.97) between predicted response probability and observed response rate (FIG. 25A). Additionally, the AUCs of the ROC curves were 0.71, 0.77 and 0.78 for the 3-, 6- and 12-month time points, respectively (FIG. 25B), demonstrating strong predictive capability of the RAP-based models over the first year of ICB-based treatment. Notably, the RAP-based model displayed superior predictive performance in comparison to the PD-L1-based model (AUCs=0.5-0.6 over the first year) and the clinical model (AUCs=0.52-0.62 over the first year) (FIG. 18).

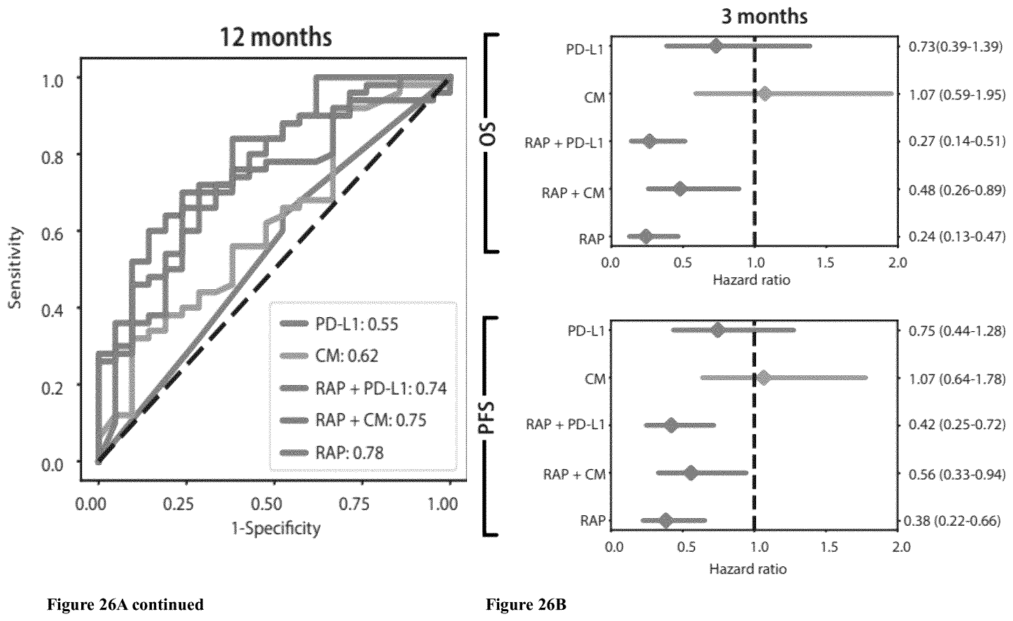

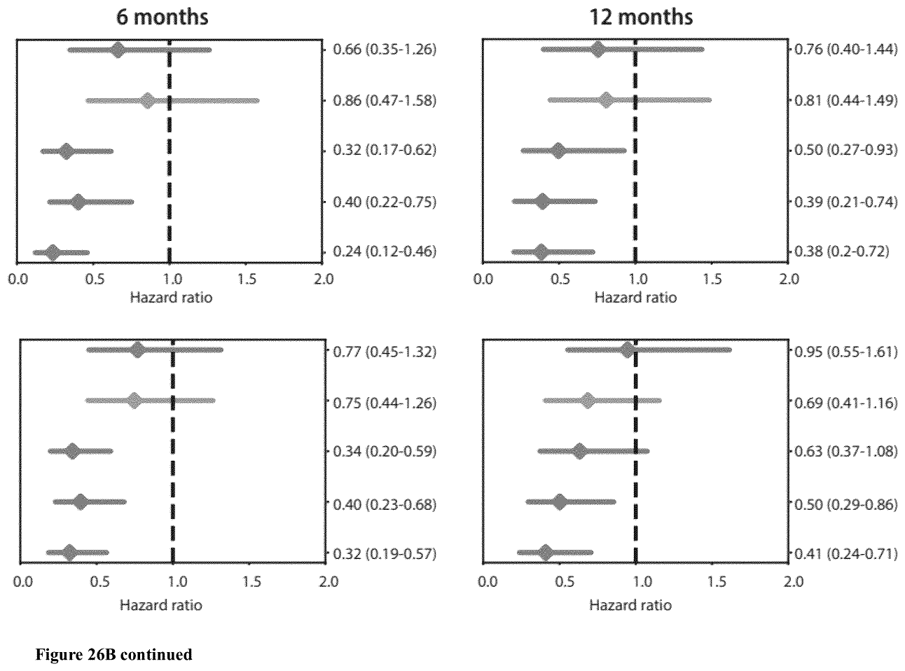

It was then tested whether integrating clinical parameters into the RAP model would improve its predictive performance. To this end, the PD-L1-based model or clinical model were integrated with the RAP-based model and predictive performance was compared. Interestingly, adding the PD-L1 parameter to the RAP-based model slightly increased predictive performance for the 6-month time point, while integrating the RAP-based and clinical models decreased predictive performance overall (FIG. 26A). In the survival analysis, the RAP model displayed the best HR in comparison to the four other models, while the HR was not significant for the PD-L1-based and clinical models (FIG. 26B).

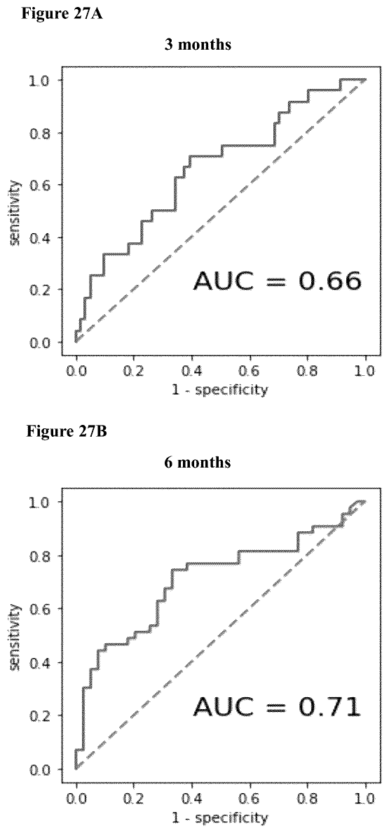

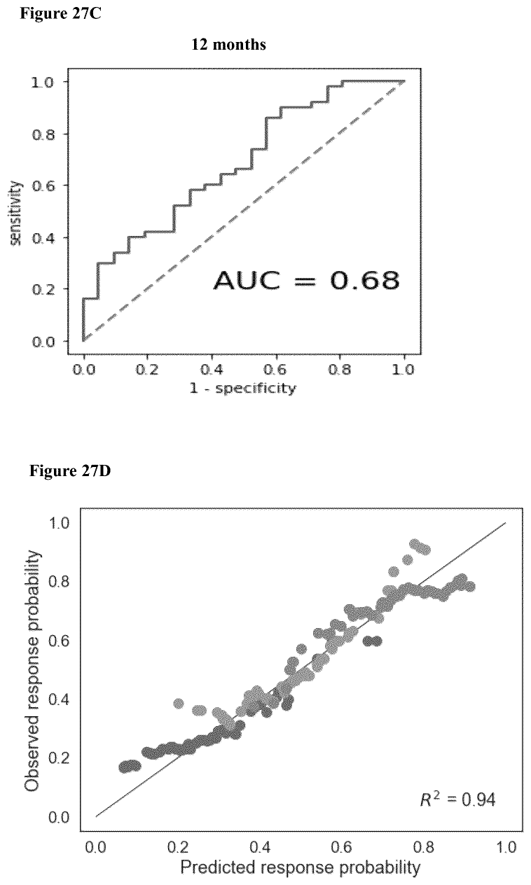

In a separate model integrating the number of active RAPs obtained according to the RAP model, PD-L1 expression and age, the AUCs of the ROC curves were 0.66, 0.71 and 0.68 for the 3-, 6- and 12-month time points, respectively (FIG. 27A-27C). Linear regression analysis demonstrated a high goodness of fit (R²=0.94) between predicted response probability and observed response rate at all time points (FIG. 27D). Thus, the combination of RAP number with clinical parameters was found to be as good or superior to the combination of total RAP score (per RAP activity levels) with clinical parameters in its ability to correctly predict patient response.

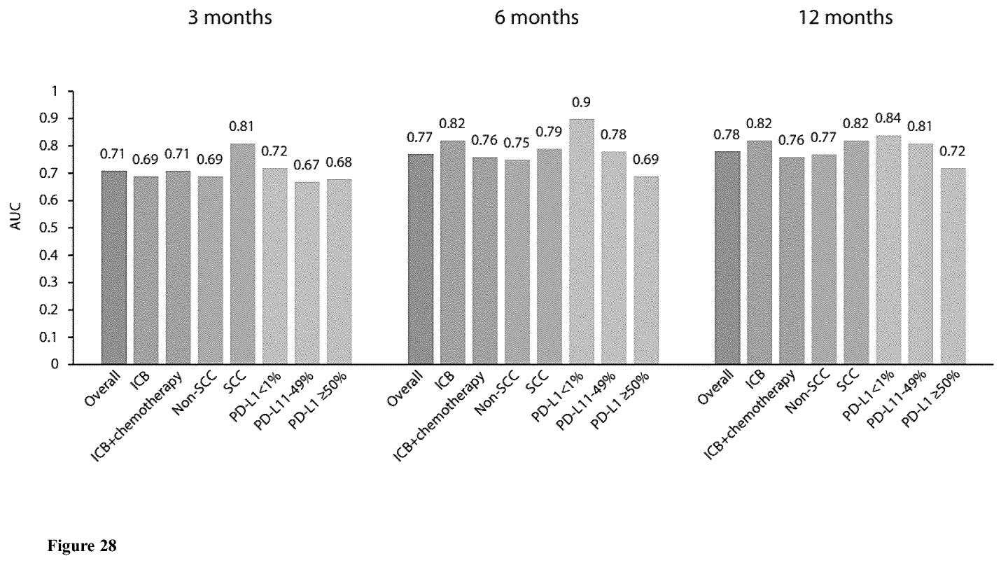

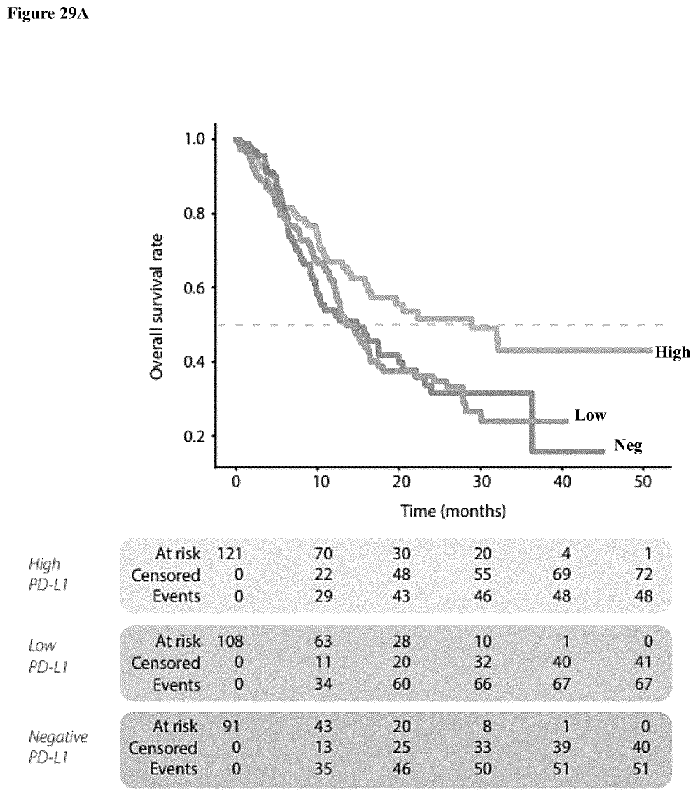

Lastly, the RAP-based model performance in different patient subsets was tested (FIG. 28). The model displayed strong predictive performance in both ICI monotherapy and ICI-chemotherapy subsets, similar to the performance in the population overall. Histology subset analysis, on the other hand, showed improved prediction for the squamous cell carcinoma subset at 3 months compared to the overall population. At 6 and 12 months, the strongest prediction was observed in the PD-L1-negative subset, while prediction was slightly weaker in the PD-L1-high subset compared to the overall population.

### Example 4: The RAP-Based Model Forecasts Differential Outcomes in PD-L1-High Patients

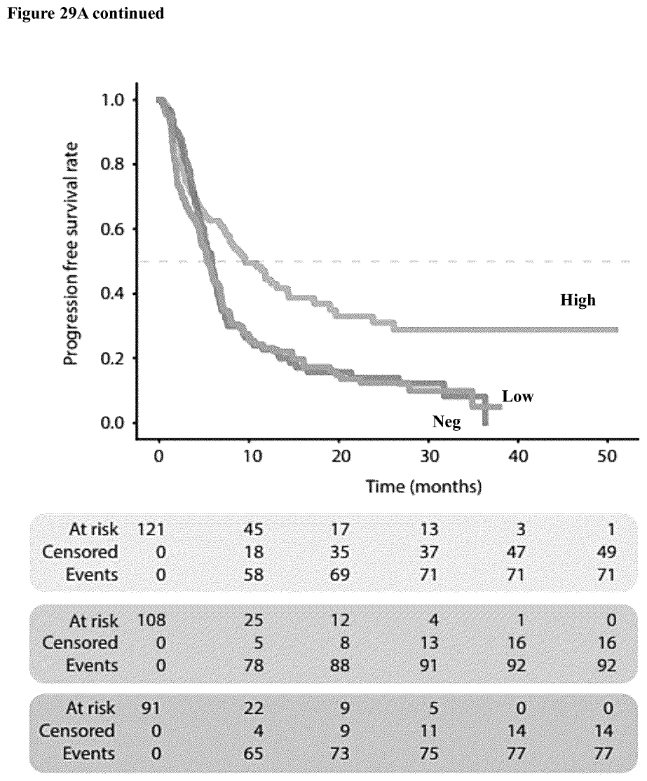

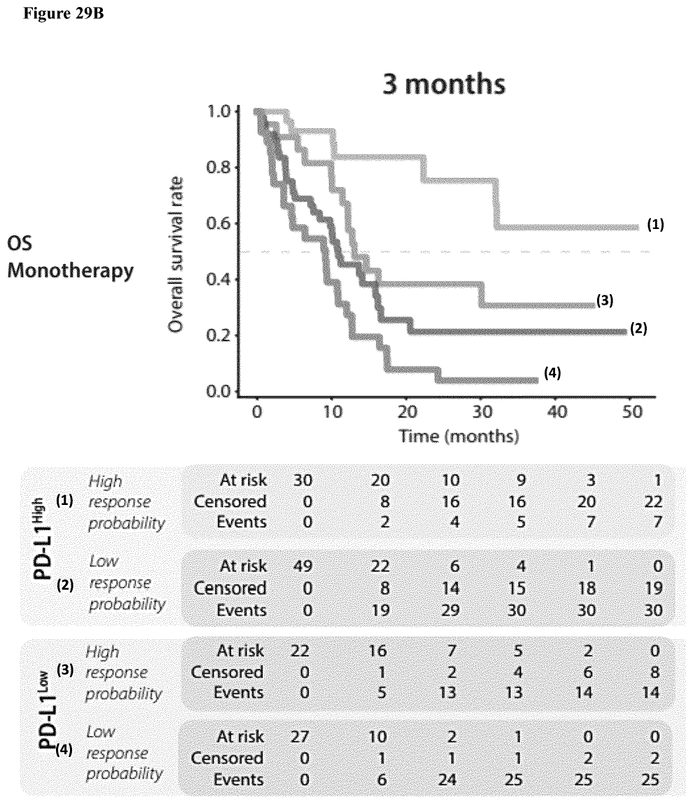

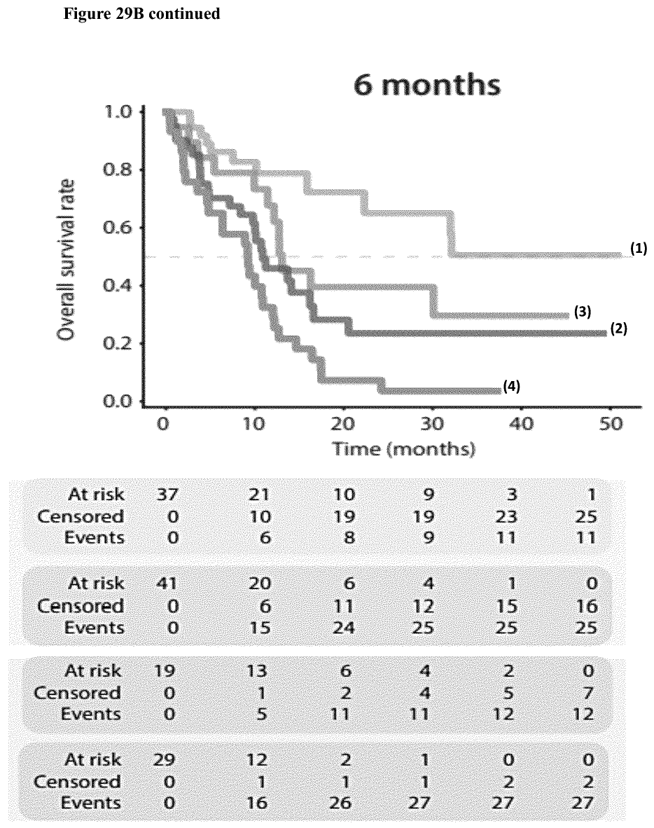

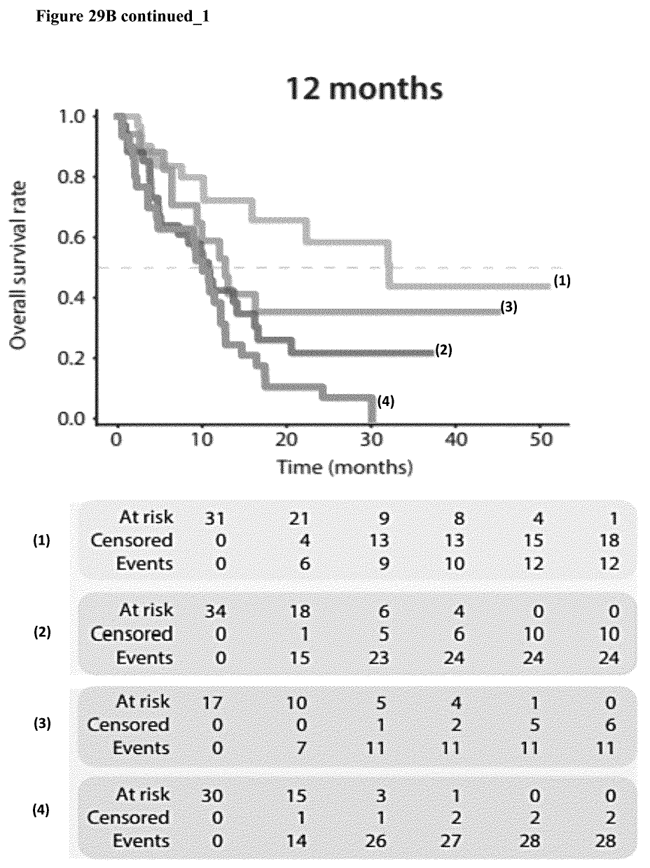

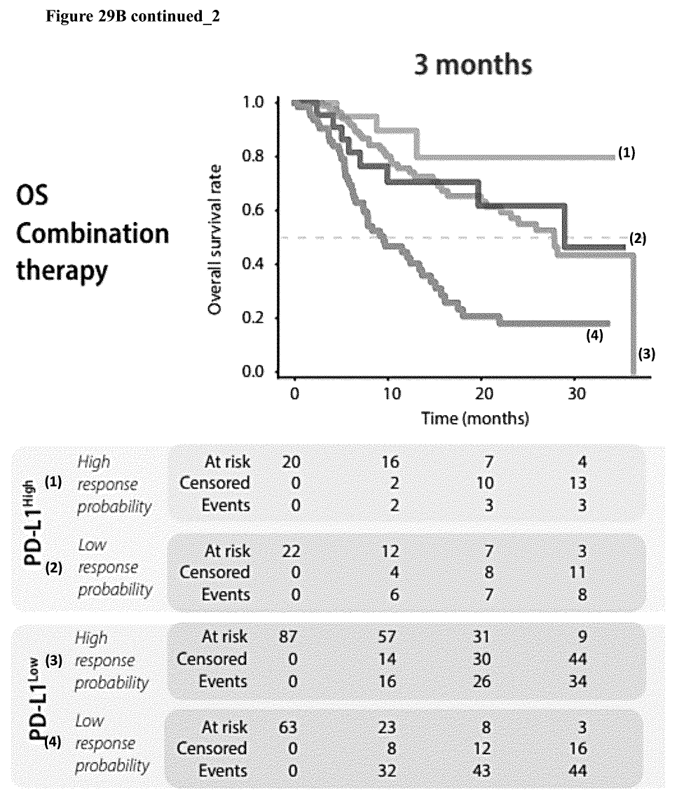

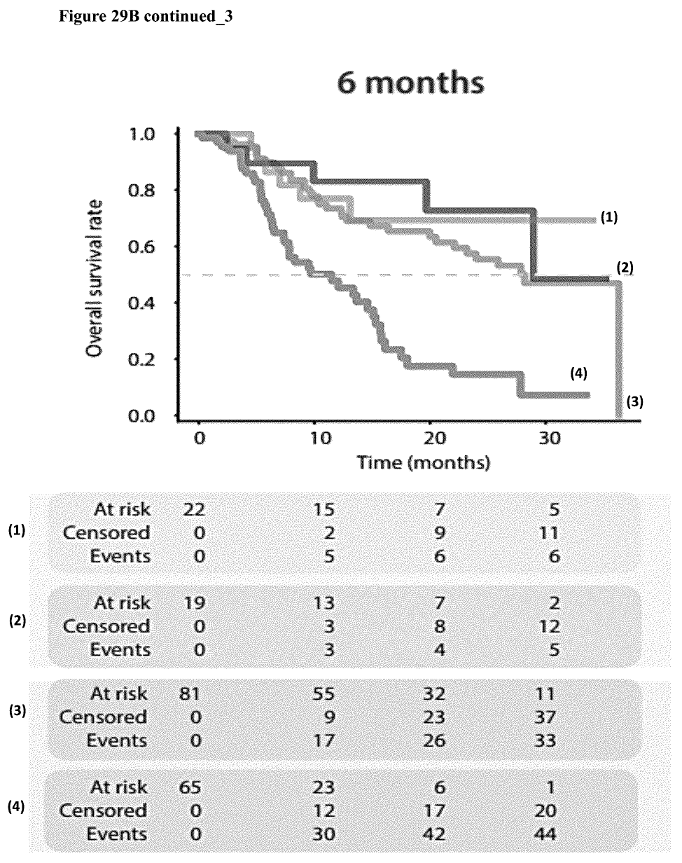

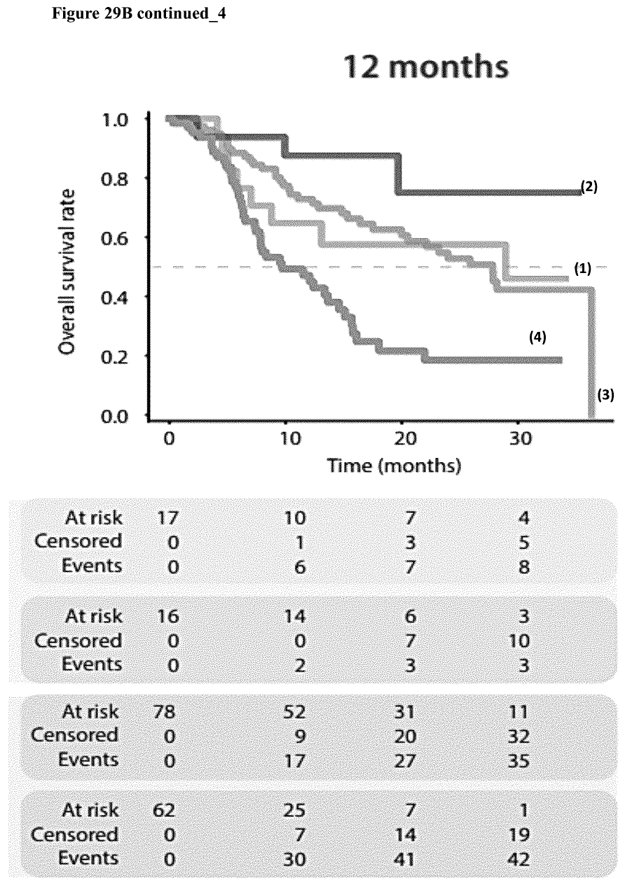

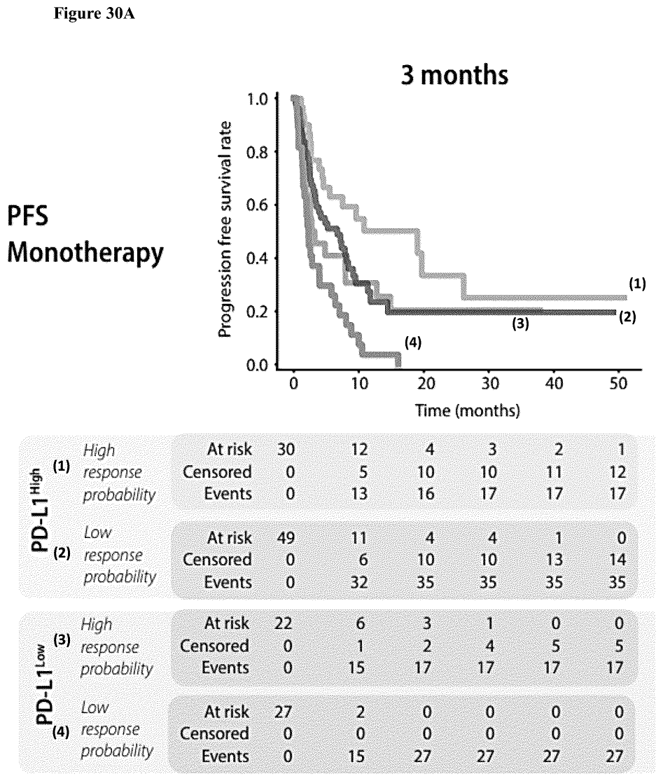

According to current guidelines for initial treatment of driver mutation-negative NSCLC, patients with PD-L1-high tumors are treated with monotherapy of ICI or combination of ICI and chemotherapy, where the latter therapy option is recommended in the case of rapidly progressive disease. For patients with PD-L1-low or PD-L1-negative tumors, combination of ICI and chemotherapy is the only recommended option. In the used cohort, PD-L1-high patients displayed better prognosis and up to two-fold difference in median OS and PFS in comparison to PD-L1-low and PD-L1-negative patients (FIG. 29A). Most PD-L1-high patients (65.3%) were treated with monotherapy ICI.

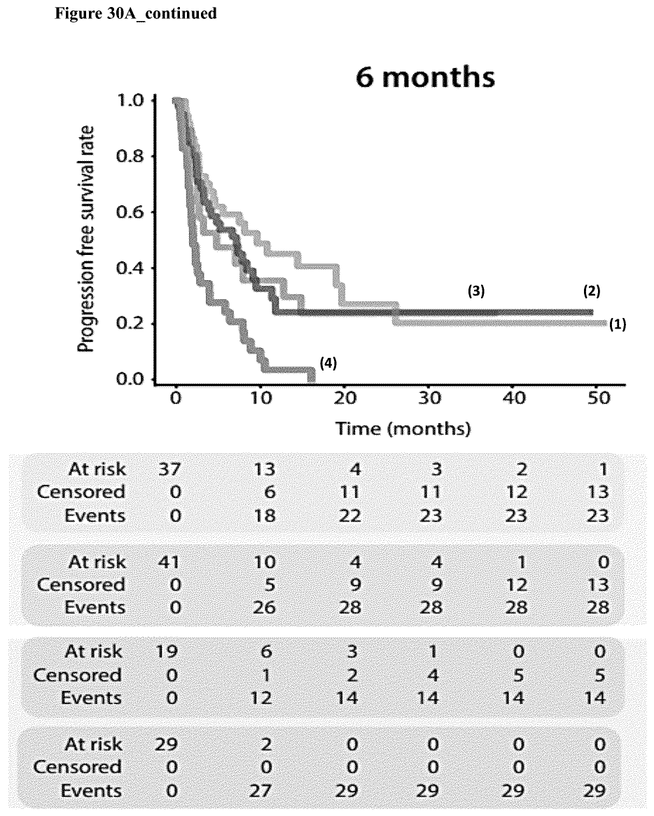

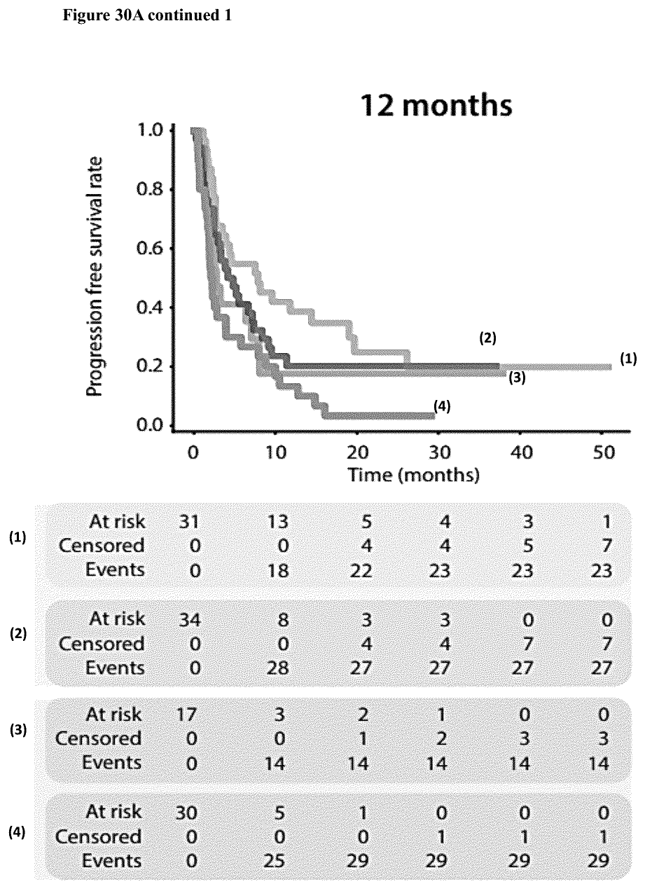

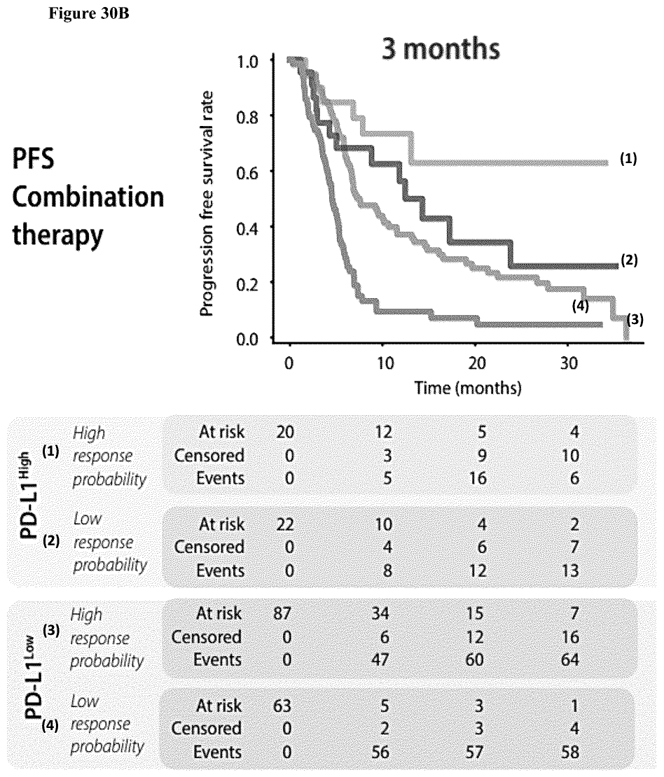

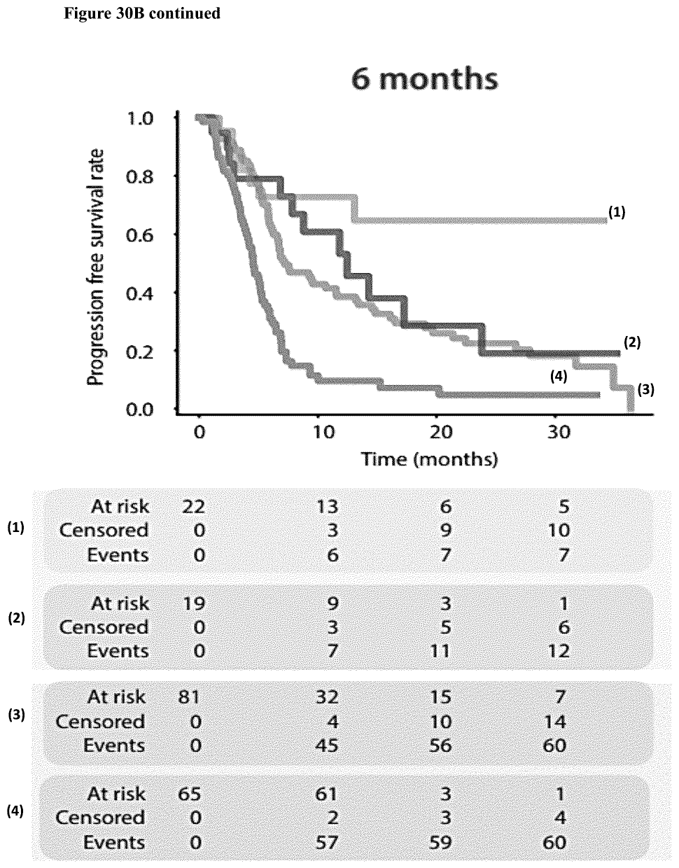

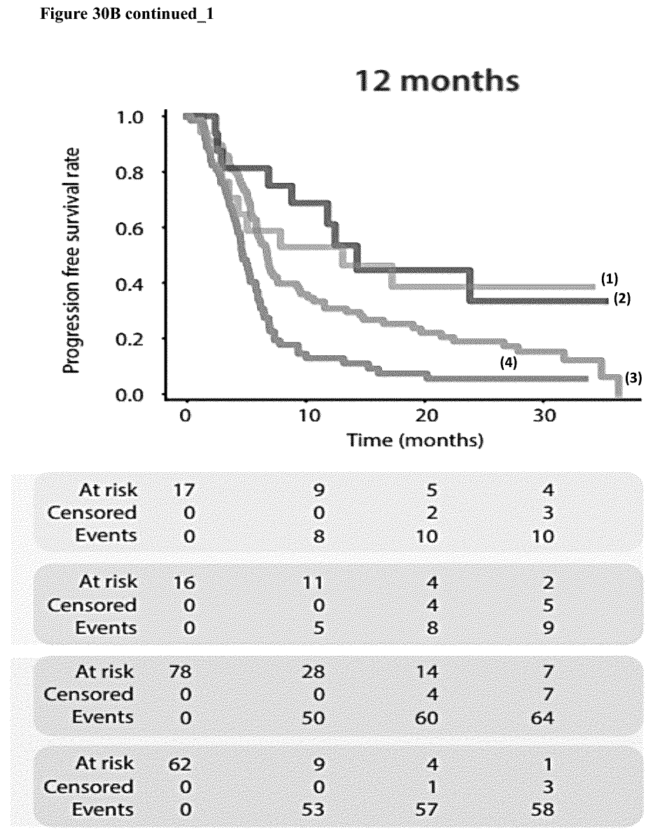

Among the PD-L1-high patients receiving monotherapy ICI, it is possible to identify patients who would have fared better with combination ICI-chemotherapy. To explore this, the ability of the RAP-based model to forecast survival outcomes in patient subsets (i.e., PD-L1-high patients receiving monotherapy ICI versus combination of ICI-chemotherapy) was tested. In the monotherapy subset, patients in the high response probability group had significantly longer OS than patients in the low response probability group at all three time points (3-month HR=0.24, p<0.001; 6-month HR=0.36, p=0.004; 12-month HR=0.42, p=0.01; FIG. 29B). Notably, high response probability patients the median OS was not reached at the 3- and 6-month time points and was 32.6 months at the 12-month time point in comparison to 10.98 months on average in low response probability patients. Notably, it exceeded the median OS of PD-L1-high patients overall (29.4 months). In contrast, in the combination therapy subset, there were no significant differences in OS at any time point when comparing high and low response probability groups (FIG. 29B). Notably, no significant differences in PFS were observed between high and low response probability groups in monotherapy or combination therapy subsets (FIG. 30A, 30B respectively). Taken together, the OS analysis demonstrates the ability of the RAP-based model to identify PD-L1-high patients that benefit less from ICI monotherapy and that may achieve better outcomes with combination of ICI and chemotherapy.

Regarding PD-L1-low/neg patients, both high and low response probability groups displayed poor prognosis in the monotherapy subset (FIG. 29B). This indicates that the model did not identify a subgroup of PD-L1-low/neg patients that would benefit from ICI monotherapy. In the combination therapy subset, the high response probability group survived significantly longer than the low response probability group (3-month HR=0.34, p<0.0001; 6-month HR=0.32, p<0.0001; 12-month HR=0.38, p<0.0001), and displayed a similar outcome to PD-L1-high patients in this subset (FIG. 29B). Thus, the RAP-based model differentiated between PD-L1-low/neg patients that benefit display longer OS from the recommended combination therapy and those that do not. However, currently there are no treatment options to address the latter case.

1. A method of predicting response of a subject suffering from a disease to a therapy and treating said subject, the method comprising:
a. receiving protein expression levels for a plurality of factors
i. in a population of subjects suffering from said disease and known to respond to said therapy (responders);
ii. in a population of subjects suffering from said disease and known to not respond to said therapy (non-responders); and
iii. in said subject;
b. calculate for factors of said plurality of factors a resistance score, where said resistance score is based on the similarity of said factor expression level in said subject to the factor expression level in said responders and the similarity of said factor expression level in said subject to said non-responders; and
c. classify a factor of said plurality of factors with a resistance score beyond a predetermined threshold as a resistance-associated factor;
wherein a subject with a number of resistance-associated factors above a predetermined number is predicted to be resistant to said therapy and a subject with a number of resistance-associated factors at or below a predetermined number is predicted to respond to said therapy; and
d. at least one of:
i. administering said therapy or continuing to administer said therapy to said subject predicted to respond to said therapy;
ii. discontinuing said therapy or not administering said therapy to said subject predicted to be resistant to said therapy; and
iii. administering an alternative therapy to said subject predicted to be resistant to said therapy:
thereby predicting the response of a subject to a therapy and treating said subject.

2. The method claim 1, the method further comprising:
d. sum the number of resistance-associated factors present for said subject; and
e. applying a trained machine learning algorithm to said number of resistance-associated factors and at least one clinical parameter of said subject, wherein said trained machine learning algorithm outputs a final resistance score and a final resistance score beyond a predetermined threshold indicates said subject is resistant to said therapy.

3. A method of predicting response of a subject suffering from a disease to a therapy, the method comprising:
a. receiving factor expression levels for a plurality of factors
i. in a population of subjects suffering from said disease and known to respond to said therapy (responders);
ii. in a population of subjects suffering from said disease and known to not respond to said therapy (non-responders); and
iii. in said subject;
b. calculate for factors of said plurality of factors a resistance score, where said resistance score is based on the similarity of said factor expression level in said subject to the factor expression level in said responders and the similarity of said factor expression level in said subject to the factor expression level said non-responders and wherein said calculating comprises applying a machine learning algorithm trained on a training set comprising said received factor expression levels in responders and non-responders and the sex of each of said responders and non-responders to individual received factor expression levels from said subject and said subject's sex and wherein said machine learning algorithm outputs said resistance score; and
c. sum said calculated resistance scores to produce a total resistance score;
wherein a subject with a total resistance score beyond a predetermined threshold is predicted to be resistant to said therapy;
thereby predicting the response of a subject to a therapy.

4. A method comprising:
at a training stage, training a machine learning algorithm on a training set comprising:
(i) the number of resistance-associated factors expressed in samples from subjects suffering from a disease and known to be responsive to a therapy and the number of resistance-associated factors expressed in samples from subjects suffering from said disease and known to be non-responsive to said therapy;
(ii) at least one clinical parameter of said subjects known to be responsive and said subjects known to be non-responsive; and
(iii) labels associated with the responsiveness of said subjects suffering from said disease;
to produce a trained machine learning algorithm, wherein said trained machine learning algorithm is trained to predict responsiveness of subjects suffering from said disease to said therapy;
(i) factor expression levels of resistance-associated factors in samples from subjects suffering from a disease and known to be responsive to a therapy and factor expression levels of resistance-associated factors in samples from subjects suffering from said disease and known to be non-responsive to said therapy; and
(ii) labels associated with the responsiveness of said subjects suffering from said disease;
to produce a trained machine learning algorithm, wherein said trained machine learning algorithm is trained to predict activity of a resistance-associated factor in a subject; or
(ii) factor expression levels of resistance-associated factors in samples from subjects suffering from a disease and known to be responsive to a therapy and factor expression levels of resistance-associated factors in samples from subjects suffering from said disease and known to be non-responsive to said therapy:
(ii) at least one clinical parameter of said subjects known to be responsive and said subjects known to be non-responsive; and
(iii) labels associated with the responsiveness of said subjects suffering from said disease;
to produce a trained machine learning algorithm, wherein said trained machine learning algorithm is trained to predict activity of a resistance-associated factor in a subject.

5. (canceled)

6. The method of claim 4, wherein at least one of:
a. said number of resistance-associated factors and said at least one clinical parameter are labeled with said labels;
b. said final resistance score predetermined threshold is 0.5 and a resistance score above 0.5 indicates the subject is resistant to the therapy or said final resistance score is converted to a response score by the equation (1-final resistance score) and wherein a response score above a predetermined threshold indicates the subject is responsive to therapy; and
c. said resistance-associated factors in each subject are determined by a method comprising:
i. receiving factor expression levels for a plurality of factors
1. in a population of subjects suffering from said disease and known to respond to said therapy (responders);
2. in a population of subjects suffering from said disease and known to not respond to said therapy (non-responders); and
3. in said each subject;
ii. calculate for each factor of said plurality of proteins a resistance score, where said resistance score is based on the similarity of said factor expression level in said each subject to the factor expression level in said responders and the similarity of said factor expression level in said subject to said non-responders; and
iii. classify a factor of said plurality of factors with a resistance score above a predetermined threshold as a resistance-associated factor.

7. (canceled)

8. (canceled)

9. The method of claim 1, wherein at least one of:
a. said method comprises before (b) selecting a subset of said plurality of factors, wherein said subset comprises factors that best differentiate between said responders and non-responders, and wherein said calculating is for each factor of said subset;
b. said calculating comprises applying a machine learning algorithm trained on a training set comprising said received factor expression levels in responders and non-responders to individual received factor expression levels from said subject and wherein said machine learning algorithm outputs said resistance score;
c. said calculating comprises calculating a mean expression for each factor in responders and non-responders and wherein said resistance score is based on the ratio of deviation of said factors expression in said subject from said calculated mean in responders to the deviation of said factors expression in said subject from said calculated mean in non-responders.

10. The method of claim 9, wherein at least one of:
a. said selecting comprises applying a statistical test to said received factor expression levels, optionally wherein said statistical test is a Kolmogorov-Smirnov test;
b. said training set further comprises the sex of each responder and non-responder and said machine learning algorithm is applied to individual received factor expression levels from said subject and said subject's sex;
c. said method further comprises performing a dimensionality reduction step with respect to said plurality of factors, to reduce the number of factors in said plurality;
d. said method further comprises performing a dimensionality reduction step with respect to said plurality of factors, to reduce the number of factors in said plurality and wherein said dimensionality reduction step identifies a subset of principal factors and said training set comprises only expression levels of said subset of principal factors, optionally wherein said subset of principal factors are the factors that most evenly balance the predicted number of responders and non-responders;
e. said predetermined threshold is determined by performing a cross-validation within said training set;
f. said calculating further comprises calculating a distribution and standard deviation for each factor in responders and non-responders and wherein said deviation is measured as a multiple of said calculated standard deviation; and
g. said resistance score is calculated by the equation monotonic
$$( \frac{\vert {Z}_{R} \vert}{\vert {z}_{N R} \vert + c} )$$
wherein Z_{R} is said deviation of said factors expression in said subject from said calculated mean in responders, Z_{NR} is said deviation of said factors expression in said subject from said calculated mean in non-responders, and c is a constant.

11. (canceled)

12. (canceled)

13. (canceled)

14. (canceled)

15. (canceled)

16. (canceled)

17. (canceled)

18. (canceled)

19. The method of claim 10, wherein said resistance score is calculated by the equation monotonic
$$( \frac{\vert {Z}_{R} \vert}{\vert {z}_{N R} \vert + c} )$$
wherein Z_{R} is said deviation of said factors expression in said subject from said calculated mean in responders, Z_{NR} is said deviation of said factors expression in said subject from said calculated mean in non-responders, and c is a constant, and said predetermined threshold for said resistance score is about 2.9, and a resistance score above 2.9 indicates a factor is a resistance-associated factor.

20. The method of claim 1, wherein at least one of:
a. said plurality of factors is at least 200 factors;
b. said protein expression levels are protein expression levels in a biological sample provided by said subjects;
c. said protein expression levels are protein expression levels in a blood plasma, whole blood, blood serum or peripheral blood mononuclear cells provided by said subjects.

21. The method of claim 1, wherein said predetermined number of resistance-associated factors is 3 and wherein a subject with more than 3 resistance-associated factors is predicted to be resistant to said therapy.

22. The method of claim 1, wherein said receiving factors expression levels for a plurality of factors comprises:
a. receiving factors expression levels for a group of factors larger than said plurality in said population of responders and said population of non-responders;
b. for each factors of said group applying a machine learning algorithm trained on said received factors expression levels in responders and non-responders;
c. selecting a subgroup of factors for which the algorithm most evenly divided the subjects in said populations into responders and non-responders; and
d. designating said subgroup of factors as said plurality of factors.

23. (canceled)

24. (canceled)

25. (canceled)

26. The method of claim 1, wherein said protein expression levels are protein expression levels in a biological sample provided by said subjects before receiving said therapy, optionally wherein before is at most 24 hours before.

27. (canceled)

28. The method of claim 1, wherein said protein expression levels are protein expression levels in a biological sample provided by said subject after receiving said therapy, optionally wherein after receiving said therapy is after receiving a first treatment with said therapy, after is at least 24 hours after or both.

29. (canceled)

30. (canceled)

31. The method of claim 1, wherein said protein expression levels are protein expression levels in a biological sample and wherein said biological sample provided by each subject in a population is the same type of biological sample, said biological samples provided by said responder population, said non-responder population and said subject are all the same type of biological sample or both.

32. (canceled)

33. The method of claim 2, wherein at least one of:
a. said trained machine learning algorithm is trained by a method comprising:
at a training stage, training a machine learning algorithm on a training set comprising:
(i) the number of resistance-associated factors expressed in samples from subjects suffering from a disease and known to be responsive to a therapy and the number of resistance-associated factors expressed in samples from subjects suffering from said disease and known to be non-responsive to said therapy;
(ii) at least one clinical parameter of said subjects known to be responsive and said subjects known to be non-responsive; and
(iii) labels associated with the responsiveness of said subjects suffering from said disease;
to produce a trained machine learning algorithm, wherein said trained machine learning algorithm is trained to predict responsiveness of subjects suffering from said disease to said therapy; and
b. said at least one clinical parameter is selected from: a subject's age, sex, line of treatment, and expression of a biomarker within a sample from a subject, optionally wherein said biomarker is PD-L1.

34. The method of claim 3, wherein said trained machine learning algorithm is trained by a method comprising:
at a training stage, training a machine learning algorithm on a training set comprising:
(i) factor expression levels of resistance-associated factors in samples from subjects suffering from a disease and known to be responsive to a therapy and factor expression levels of resistance-associated factors in samples from subjects suffering from said disease and known to be non-responsive to said therapy;
(ii) at least one clinical parameter of said subjects known to be responsive and said subjects known to be non-responsive; and
(iii) labels associated with the responsiveness of said subjects suffering from said disease;
to produce a trained machine learning algorithm, wherein said trained machine learning algorithm is trained to predict activity of a resistance-associated factor in a subject; or
at a training stage, training a machine learning algorithm on a training set comprising:
factor expression levels of resistance-associated factors in samples from subjects (ii) suffering from a disease and known to be responsive to a therapy and factor expression levels of resistance-associated factors in samples from subjects suffering from said disease and known to be non-responsive to said therapy; and
(ii) labels associated with the responsiveness of said subjects suffering from said disease;
to produce a trained machine learning algorithm, wherein said trained machine learning algorithm is trained to predict activity of a resistance-associated factor in a subject.

35. The method of claim 1, wherein said disease is cancer.

36. The method of claim 1, wherein said therapy is immune checkpoint inhibition, optionally wherein said immune checkpoint inhibition inhibits the PD-1/PD-L1 axis.

37. (canceled)

38. (canceled)

39. The method of claim 33, further comprising at an inference stage, receiving as input, the number of resistance-associated factors expressed in a sample from a subject suffering from said disease and with unknown responsiveness to said therapy, and at least one clinical parameter of said subject with unknown responsiveness and applying said trained machine learning algorithm to said received input, to predict responsiveness to said therapy of said subject with unknown responsiveness.

40. (canceled)

41. (canceled)

42. (canceled)

43. The method of claim 1, further wherein said alternative therapy comprises administering to said subject predicted to be resistant to said therapy, said therapy in combination with an agent that modulates at least one of said resistance-associated factors or a factor in a functional pathway comprising said at least one resistance-associated factor, wherein:
a. said at least one resistance-associated factor is more highly expressed in said non-responders than said responders and increases activity of said pathway and said agent inhibits said resistance-associated factor or said pathway;
b. said at least one resistance-associated factor is more highly expressed in said non-responders than said responders and decreases activity of said pathway and said agent inhibits said resistance-associated factor or activates said pathway;
c. said at least one resistance-associated factor is more lowly expressed in said non-responders than said responders and increases activity of said pathway and said agent activates said resistance-associated factor or said pathway; or
d. said at least one resistance-associated factor is more lowly expressed in said non-responders than said responders and decreases activity of said pathway and said agent activates said resistance-associated factor or inhibits said pathway.
